(19) United States
(12) Patent Application Publication
(10) Pub. No.: US 2003/0199567 A1
(43) Pub. Date: Oct. 23, 2003

(54) Combinations of an alpha-2-delta ligand with a selective inhibitor of cyclooxygenase-2

(76) Inventor: Charles Price Taylor JR., Chelsea, MI (US)

Correspondence Address: WARNER-LAMBERT COMPANY, 2800 PLYMOUTH RD, ANN ARBOR, MI, 48105, US

(21) Appl. No.: 10/366,798

(22) Filed: Feb. 14, 2003

Related U.S. Application Data
(60) Provisional application No. 60/359,295, filed on Feb. 22, 2002.
(60) Provisional application No. 60/404,365, filed on Aug. 19, 2002.

(51) Int. Cl. A61K 31/415; A61K 31/195

(52) U.S. Cl. 514/406; 514/561

(57) ABSTRACT

The invention relates to a combination, comprising a selective inhibitor of COX-2, or a pharmaceutically acceptable salt thereof, and an Alpha-2-delta ligand, or a pharmaceutically acceptable salt thereof, and valdecoxib. Examples of selective inhibitors of COX-2 include valdecoxib, rofecoxib, and celecoxib. Examples of Alpha-2-delta ligands include gabapentin, pregabalin, (3S,4S)-(1-Aminomethyl-3,4-dimethyl-cyclopentyl)-acetic acid, and 3-(1-aminomethyl-cyclohexylmethyl)-4H-[1,2,4]oxadiazol-5-one hydrochloride. The combinations are useful for treating certain diseases including cartilage damage, inflammation, pain, and arthritis.

### CROSS-REFERENCE TO RELATED APPLICATIONS

This application claims benefit of priority from U.S. Provisional Application No. 60/359,295, filed Feb. 22, 2002, and No. 60/404,365, filed Aug. 19, 2002, which are hereby incorporated herein by reference.

### BACKGROUND OF THE INVENTION

This invention relates to combinations that comprise a selective inhibitor of COX-2 and an Alpha-2-delta ligand, or pharmaceutically acceptable salts thereof. The combinations are useful for the treatment of diseases such as inflammation and pain.

More than 23 million Americans have some form of arthritis. Among the various forms of arthritis, osteoarthritis (“OA”) is the most prevalent, affecting 21 million Americans. Characterized by the degeneration of joint cartilage and adjacent bone, OA typically is an asymptomatic chronic disorder. Later, some OA patients may experience joint pain or stiffness. Rheumatoid arthritis (“RA”), which affects more than 2.1 million Americans, is an autoimmune disease that affects joint lining, cartilage and bones.

Aspirin and conventional nonsteroidal anti-inflammatory drugs (NSAIDs) such as ibuprofen, diclofenac, and naproxen are the primary agents used to treat OA- and RA-related pain. These agents inhibit prostaglandin release by blocking cyclooxygenase-mediated conversion of cell membrane lipids from arachidonic acid.

Two forms of COX are now known, a constitutive isoform usually named cyclooxygenase-1 (“COX-1”) and an inducible isoform usually named cyclooxygenase-2 (“COX-2”), the latter of which expression is upregulated at sites of inflammation. COX-1 appears to play a physiological role and to be responsible for gastrointestinal and renal protection. On the other hand, COX-2 appears to play a pathological role and is believed to be the predominant isoform present in inflammation conditions. The therapeutic use of conventional COX inhibitors, which are typically nonselective inhibitors of both COX—I and COX-2, is limited due to drug associated side effects, including life threatening ulceration and renal toxicity. Compounds that selectively inhibit COX-2 would exert anti-inflammatory effects without the adverse side effects associated with COX-1 inhibition.

Valdecoxib is a COX-2 specific inhibitor that was approved in 2001 by the United States Food and Drug Administration (“FDA”) for treating the signs and symptoms of osteoarthritis (OA) and adult rheumatoid arthritis (RA); and the treatment of pain associated with menstrual cramping. Valdecoxib tablets are marketed under the tradename BEXTRA®. In a combined analysis of various clinical studies with valdecoxib, valdecoxib was well tolerated with an overall upper gastrointestinal safety profile (ulcers, perforations, obstructions and GI bleeds) significantly better than the conventional NSAIDs studied such as ibuprofen, diclofenac and naproxen.

Alpha-2-delta ligands, including gabapentin, pregabalin, and 3-(1-aminomethyl-cyclohexylmethyl)-4H-[1,2,4]oxadiazol-5-one hydrochloride have also been found to be effective for treating inflammation and pain. Specifically, it is shown herein below that an Alpha-2-delta ligand is useful for inhibiting cartilage damage in a joint, and thus effective in treating underlying disease progression in osteoarthritis. Gabapentin has previously been approved by the FDA and is currently marketed under the tradename NEURONTIN® for the treatment of epilepsy and clinically for the treatment of neuropathic pain. Pregabalin and 3-(1-aminomethyl-cyclohexylmethyl)-4H-[1,2,4]oxadiazol-5-one hydrochloride are also in clinical trials for the treatment of convulsions and analgesia, respectively.

Applicant's discovery-disclosed in the instant application-that a combination of an Alpha-2-delta ligand, or a pharmaceutically acceptable salt thereof, with valdecoxib is useful for treating cartilage damage, osteoarthritis, inflammation, and pain in a mammal has not been previously disclosed. All that is required to treat cartilage damage, osteoarthritis, inflammation or pain in a mammal according to the invention is to administer to the mammal in need of treatment a therapeutically effective amount of a combination, wherein the combination comprises an Alpha-2-delta ligand and valdecoxib, or an Alpha-2-delta ligand and another selective inhibitor of COX-2, or independently selected pharmaceutically acceptable salts thereof.

### SUMMARY OF THE INVENTION

This invention provides a combination, comprising a selective inhibitor of COX-2, or a pharmaceutically acceptable salt thereof, and an Alpha-2-delta ligand, or a pharmaceutically acceptable salt thereof.

Another invention embodiment is a combination, comprising rofecoxib, or a pharmaceutically acceptable salt thereof, and an Alpha-2-delta ligand, or a pharmaceutically acceptable salt thereof.

Another invention embodiment is a combination, comprising celecoxib, or a pharmaceutically acceptable salt thereof, and an Alpha-2-delta ligand, or a pharmaceutically acceptable salt thereof.

Another invention embodiment is a combination, comprising parecoxib, or a pharmaceutically acceptable salt thereof, and an Alpha-2-delta ligand, or a pharmaceutically acceptable salt thereof.

Another invention embodiment is a combination, comprising valdecoxib, or a pharmaceutically acceptable salt thereof, and an Alpha-2-delta ligand, or a pharmaceutically acceptable salt thereof.

Another invention embodiment is a pharmaceutical composition, comprising a combination of valdecoxib and an Alpha-2-delta ligand, or a pharmaceutically acceptable salt thereof, and a pharmaceutically acceptable carrier, diluent, or excipient.

Another invention embodiment is a method of treating cartilage damage in a mammal in need thereof, comprising administering to the mammal a therapeutically effective amount of a combination comprising valdecoxib and an Alpha-2-delta ligand, or a pharmaceutically acceptable salt thereof.

Another invention embodiment is a method of treating inflammation in a mammal in need thereof, comprising administering to the mammal a therapeutically effective amount of a combination comprising valdecoxib and an Alpha-2-delta ligand, or a pharmaceutically acceptable salt thereof.

Another invention embodiment is a method of treating osteoarthritis in a mammal in need thereof, comprising administering to the mammal a therapeutically effective amount of a combination comprising valdecoxib and an Alpha-2-delta ligand, or a pharmaceutically acceptable salt thereof.

Another invention embodiment is a method of treating rheumatoid arthritis in a mammal in need thereof, comprising administering to the mammal a therapeutically effective amount of a combination comprising valdecoxib and an Alpha-2-delta ligand, or a pharmaceutically acceptable salt thereof.

Another invention embodiment is a method of treating psoriatic arthritis in a mammal in need thereof, comprising administering to the mammal a therapeutically effective amount of a combination comprising valdecoxib and an Alpha-2-delta ligand, or a pharmaceutically acceptable salt thereof.

Another invention embodiment is a method of treating pain in a mammal in need thereof, comprising administering to the mammal a therapeutically effective amount of a combination comprising valdecoxib and an Alpha-2-delta ligand, or a pharmaceutically acceptable salt thereof.

Other invention embodiments include:

1. A combination, comprising valdecoxib, or a pharmaceutically acceptable salt thereof, and an Alpha-2-delta ligand, or a pharmaceutically acceptable salt thereof, that is not a compound of Formulas

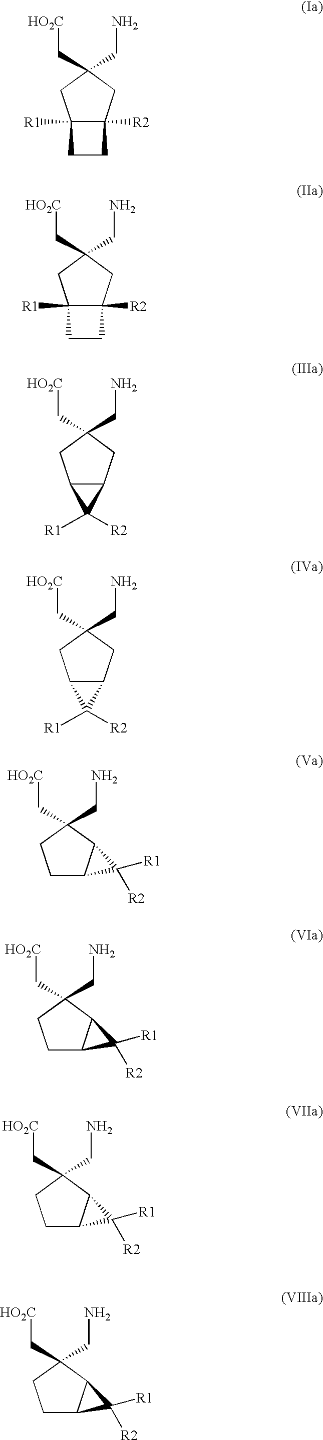

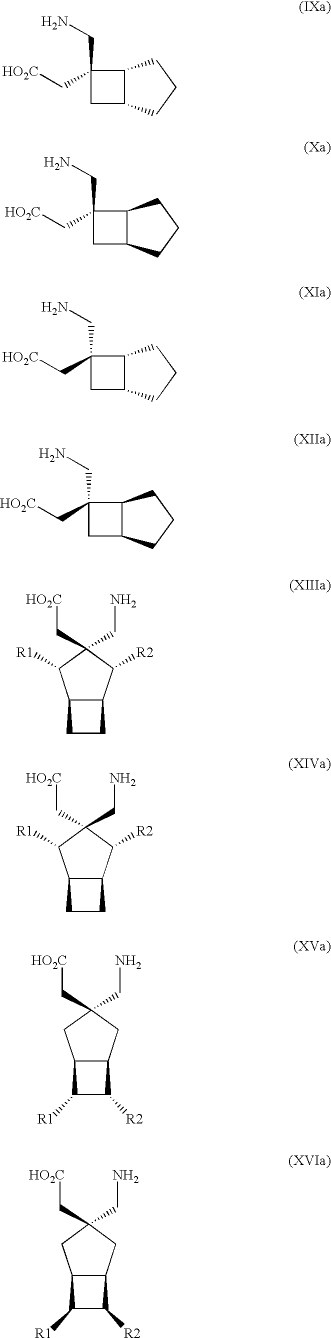

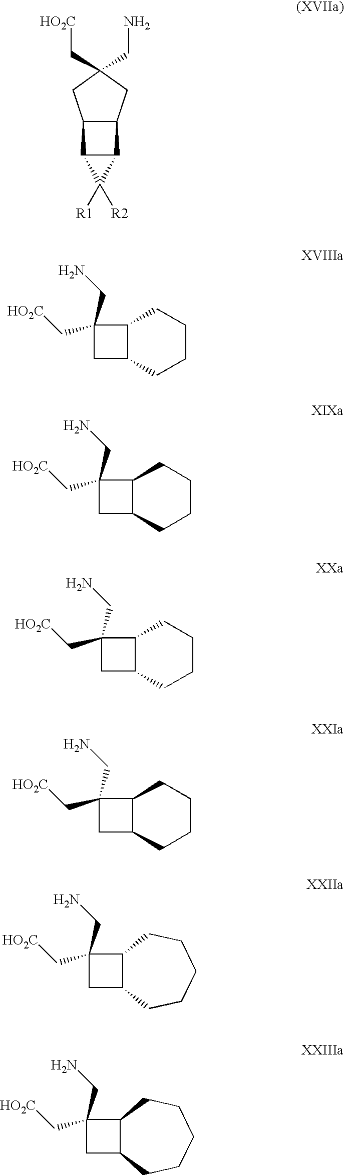

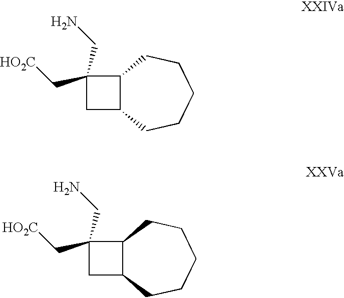

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

2. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formula I

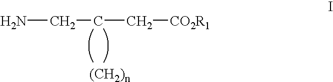

or a pharmaceutically acceptable salt thereof, wherein R₁ is hydrogen or straight or branched lower alkyl, and n is an integer of from 4 to 6.

3. The combination according to Embodiment 2, wherein the Alpha-2-delta ligand is gabapentin.

4. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formula II

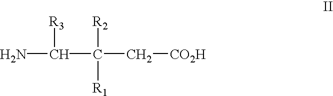

or pharmaceutically acceptable salt thereof, wherein:

R₁ is straight or branched unsubstituted alkyl of from 1 to 6 carbon atoms, unsubstituted phenyl, or unsubstituted cycloalkyl of from 3 to 6 carbon atoms;

R₂ is hydrogen or methyl; and

R₃ is hydrogen, methyl, or carboxyl.

5. The combination according to Embodiment 4, wherein the Alpha-2-delta ligand is pregabalin.

6. The combination according to Embodiment 4, wherein the Alpha-2-delta ligand is a compound named R-(3)-(aminomethyl)-5-methylhexanoic acid, or a pharmaceutically acceptable salt thereof.

7. The combination according to Embodiment 4, wherein the Alpha-2-delta ligand is a compound named 3-(1-aminoethyl)-5-methylheptanoic acid or 3-(1-aminoethyl)-5-methylhexanoic acid, or a pharmaceutically acceptable salt thereof.

8. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formula

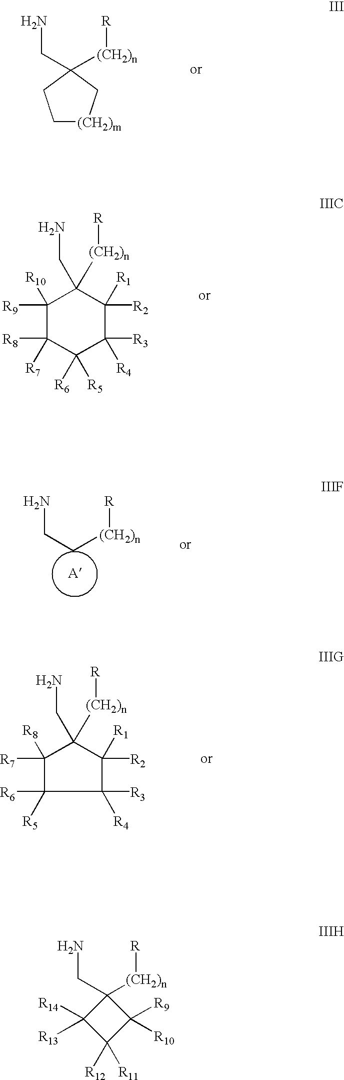

or a pharmaceutically acceptable salt thereof wherein:

n is an integer of from 0 to 2;

m is an integer of from 0 to 3;

R is sulfonamide,

amide,

phosphonic acid,

heterocycle,

sulfonic acid, or

hydroxamic acid;

R₁ to R₁₄ are each independently selected from hydrogen or straight or branched alkyl of from 1 to 6 carbons, unsubstituted or substituted benzyl or phenyl which substituents are selected from halogen, alkyl, alkoxy, hydroxy, carboxy, carboalkoxy, trifluoromethyl, and nitro;

A′ is a bridged ring selected from

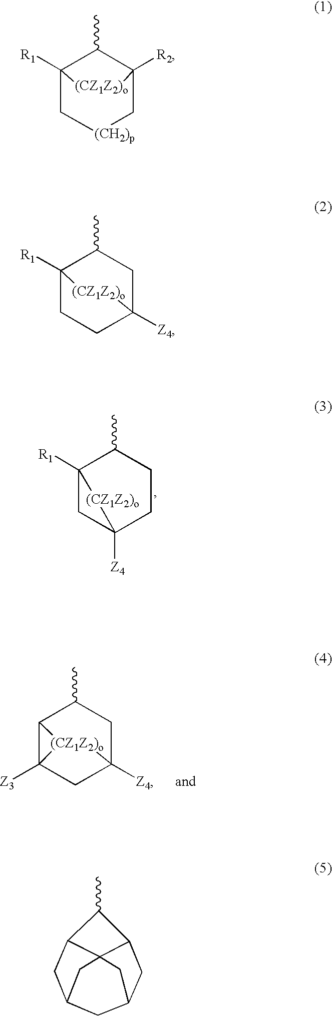

wherein

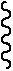

is the point of attachment;

Z₁ to Z₄ are each independently selected from hydrogen and methyl;

o is an integer of from 1 to 4; and

p is an integer of from 0 to 2 with the proviso that in formula 1 R is not —SO₃H when m is 2 and n is 1.

9. The combination according to Embodiment 8, wherein the Alpha-2-delta ligand is a compound of Formula III

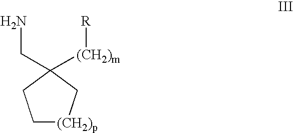

or pharmaceutically acceptable salt thereof, wherein:

m is an integer of from 0 to 2;

p is an integer of from 0 to 3; and

R is sulfonamide,

amide,

phosphonic acid,

heterocycle,

sulfonic acid, or

hydroxamic acid.

10. The combination according to Embodiment 8, wherein the Alpha-2-delta ligand is a compound of Formula III

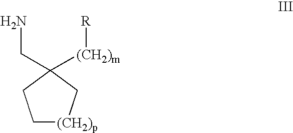

or pharmaceutically acceptable salt thereof, wherein:

m is an integer of from 0 to 2;

p is an integer of 2; and

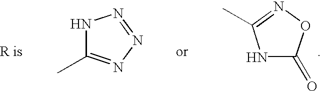

11. The combination according to Embodiment 8, wherein the Alpha-2-delta ligand is a compound named 3-(1-aminomethyl-cyclohexylmethyl)-4H-[1,2,4]oxadiazol-5-one, or a pharmaceutically acceptable salt thereof.

12. The combination according to Embodiment 8, wherein the Alpha-2-delta ligand is a compound named 3-(1-aminomethyl-cyclohexylmethyl)-4H-[1,2,4]oxadiazol-5-one hydrochloride.

13. The combination according to Embodiment 8, wherein the Alpha-2-delta ligand is a compound named 3-(1-aminomethyl-cycloheptylmethyl)-4H-[1,2,4]oxadiazol-5-one, or a pharmaceutically acceptable salt thereof.

14. The combination according to Embodiment 8, wherein the Alpha-2-delta ligand is a compound named 3-(1-aminomethyl-cycloheptylmethyl)-4H-[1,2,4]oxadiazol-5-one hydrochloride.

15. The combination according to Embodiment 8, wherein the Alpha-2-delta ligand is a compound named C-[1-(1H-tetrazol-5-ylmethyl)-cycloheptyl]-methylamine, or a pharmaceutically acceptable salt thereof.

16. The combination according to Embodiment 8, wherein the Alpha-2-delta ligand is a compound named C-[1-(1H-tetrazol-5-ylmethyl)-cycloheptyl]-methylamine.

17. The combination according to Embodiment 8, wherein the Alpha-2-delta ligand is a compound of Formulas III, IIIC, IIIF, IIIG, or IIIH, or a pharmaceutically acceptable salt thereof, wherein R is a sulfonamide selected from —NHSO₂R¹⁵ or —SO₂NHR¹⁵ wherein R¹⁵ is straight or branched alkyl or trifluoromethyl.

18. The combination according to Embodiment 8, wherein the Alpha-2-delta ligand is a compound of Formulas III, IIIC, IIIF, IIIG, or IIIH, or a pharmaceutically acceptable salt thereof, named N-[2-(1-aminomethyl-cyclohexyl)-ethyl]-methanesulfonamide, or a pharmaceutically acceptable salt thereof.

19. The combination according to Embodiment 8, wherein the Alpha-2-delta ligand is a compound of Formulas III, IIIC, IIIF, IIIG, or IIIH, or a pharmaceutically acceptable salt thereof, wherein R is a phosphonic acid, —PO₃H₂.

20. The combination according to Embodiment 8, wherein the Alpha-2-delta ligand is a compound of Formulas III, IIIC, IIIF, IIIG, or IIIH, or a pharmaceutically acceptable salt thereof, and selected from (1-aminomethyl-cyclohexylmethyl)-phosphonic acid and (2-aminomethyl-4-methyl-pentyl)-phosphonic acid, or a pharmaceutically acceptable salt thereof.

21. The combination according to Embodiment 8, wherein the Alpha-2-delta ligand is a compound of Formulas III, IIIC, IIIF, IIIG, or IIIH, or a pharmaceutically acceptable salt thereof, wherein R is a heterocycle selected from

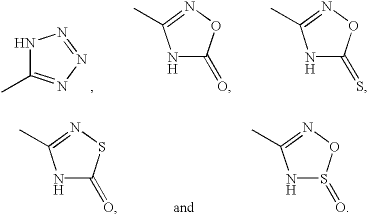

22. The combination according to Embodiment 8, wherein the Alpha-2-delta ligand is a compound of Formulas III, IIIC, IIIF, IIIG, or IIIH, or a pharmaceutically acceptable salt thereof, and selected from C-[1-(1H-tetrazol-5-ylmethyl)cyclohexyl]-methylamine, and 4-methyl-2-(1H-tetrazol-5-ylmethyl)-pentylamine, or a pharmaceutically acceptable salt thereof.

23. The combination according to Embodiment 8, wherein the Alpha-2-delta ligand is a compound of Formulas III, IIIC, IIIF, IIIG, or IIIH, or a pharmaceutically acceptable salt thereof, and selected from:

(1-Aminomethyl-cyclohexylmethyl)-phosphonic acid;

(1R-trans)(1-Aminomethyl-3-methyl-cyclohexylmethyl)-phosphonic acid;

(trans)(1-Aminomethyl-3,4-dimethyl-cyclopentylnmethyl)-phosphonic acid;

(1R-trans)(1-Aminomethyl-3-methyl-cyclopentylmethyl)-phosphonic acid;

(1S-cis)(1-Aminomethyl-3-methyl-cyclopentylmethyl)-phosphonic acid;

(1S-trans)(1-Aminomethyl-3-methyl-cyclopentylmethyl)-phosphonic acid;

(1R-cis)(1-Aminomethyl-3-methyl-cyclopentylmethyl)-phosphonic acid;

(1α,3α,4α)(1-Aminomethyl-3,4-dimethyl-cyclopentylmethyl)-phosphonic acid;

(1α,3β,4β)(1-Aminomethyl-3,4-dimethyl-cyclopentylmethyl)-phosphonic acid;

(R)(1-Aminomethyl-3,3-dimethyl-cyclopentylmethyl)-phosphonic acid;

(S)(1-Aminomethyl-3,3-dimethyl-cyclopentylmethyl)-phosphonic acid;

(1-Aminomethyl-3,3-dimethyl-cyclobutylmethyl)-phosphonic acid;

2-(1-Aminomethyl-cyclohexyl)-N-hydroxy-acetamide;

(1S-trans)2-(1-Aminomethyl-3-methyl-cyclohexyl)-N-hydroxy-acetamide;

(trans)2-(1-Aminomethyl-3,4-dimethyl-cyclopentyl)-N-hydroxy-acetamide;

(1S-cis)2-(1-Aminomethyl-3-methyl-cyclopentyl)-N-hydroxy-acetamide;

(1R-trans)2-(1-Aminomethyl-3-methyl-cyclopentyl)-N-hydroxy-acetamide;

(1R-cis)2-(1-Aminomethyl-3-methyl-cyclopentyl)-N-hydroxy-acetamide;

(1S-trans)2-(1-Aminomethyl-3-methyl-cyclopentyl)-N-hydroxy-acetamide;

(1α,3α,4α)2-(1-Aminomethyl-3,4-dimethyl-cyclopentyl)-N-hydroxy-acetamide;

(1α,3β,4β)2-(1-Aminomethyl-3,4-dimethyl-cyclopentyl)-N-hydroxy-acetamide;

(S)2-(1-Aminomethyl-3,3-dimethyl-cyclopentyl)-N-hydroxy-acetamide;

(R)2-(1-Aminomethyl-3,3-dimethyl-cyclopentyl)-N-hydroxy-acetamide;

2-(1-Aminomethyl-3,3-dimethyl-cyclobutyl)-N-hydroxy-acetamide;

N-[2-(1-Aminomethyl-cyclohexyl)-ethyl]-methanesulfonamide;

(1S-cis)N-[2-(1-Aminomethyl-3-methyl-cyclohexyl)-ethyl]-methanesulfonamide;

(trans)N-[2-(1-Aminomethyl-3,4-dimethyl-cyclopentyl)-ethyl]-methanesulfonamide;

(1S-cis)N-[2-(1-Aminomethyl-3-methyl-cyclopentyl)-ethyl]-methanesulfonamide;

(1R-trans)N-[2-(1-Aminomethyl-3-methyl-cyclopentyl)-ethyl]-methanesulfonamide;

(1R-cis)N-[2-(1-Aminomethyl-3-methyl-cyclopentyl)-ethyl]-methanesulfonamide;

(1S-cis)N-[2-(1-Aminomethyl-3-methyl-cyclopentyl)-ethyl]-methanesulfonamide;

(1α,3α,4α)N-[2-(1-Aminomethyl-3,4-dimethyl-cyclopentyl)-ethyl]-methanesulfonamide;

(1α,3β,4β)N-[2-(1-Aminomethyl-3,4-dimethyl-cyclopentyl)-ethyl]-methanesulfonamide;

(S)N-[2-(1-Aminomethyl-3,3-dimethyl-cyclopentyl)-ethyl]-methanesulfonamide;

(R)N-[2-(1-Aminomethyl-3,3-dimethyl-cyclopentyl)-ethyl]-methanesulfonamide;

N-[2-(1-Aminomethyl-3,3-dimethyl-cyclobutyl)-ethyl]-methanesulfonamide;

(1S-cis)3-(1-Aminomethyl-3-methyl-cyclohexylmethyl)-4H-[1,2,4]oxadiazol-5-one;

(trans)3-(1-Aminomethyl-3,4-dimethyl-cyclopentylmethyl)-4H-[1,2,4]oxadiazol-5-one;

(1S-cis)3-(1-Aminomethyl-3-methyl-cyclopentylmethyl)-4H-[1,2,4]oxadiazol-5-one;

(1R-trans)3-(1-Aminomethyl-3-methyl-cyclopentylmethyl)-4H-[1,2,4]oxadiazol-5-one;

(1R-cis)3-(1-Aminomethyl-3-methyl-cyclopentylmethyl)-4H-[1,2,4]oxadiazol-5-one;

(1S-trans)3-(1-Aminomethyl-3-methyl-cyclopentylmethyl)-4H-[1,2,4]oxadiazol-5-one;

(1α,3α,4α)3-(1-Aminomethyl-3,4-dimethyl-cyclopentylmethyl)-4H-[1,2,4]oxadiazol-5-one;

(1α,3β,4β)3-(1-Aminomethyl-3,4-dimethyl-cyclopentylmethyl)-4H-[1,2,4]oxadiazol-5-one;

(S)3-(1-Aminomethyl-3,3-dimethyl-cyclopentylmethyl)-4H-[1,2,4]oxadiazol-5-one;

(R)3-(1-Aminomethyl-3,3-dimethyl-cyclopentylmethyl)-4H-[1,2,4]oxadiazol-5-one;

3-(1-Aminomethyl-3,3-dimethyl-cyclobutylmethyl)-4H-[1,2,4]oxadiazol-5-one;

3-(1-Aminomethyl-cyclohexylmethyl)-4H-[1,2,4]oxadiazole-5-thione;

(1S-cis)3-(1-Aminomethyl-3-methyl-cyclohexylmethyl)-4H-[1,2,4]oxadiazole-5-thione;

(trans)3-(1-Aminomethyl-3,4-dimethyl-cyclopentylmethyl)-4H-[1,2,4]oxadiazole-5-thione;

(1S-cis)3-(1-Aminomethyl-3-methyl-cyclopentylmethyl)-4H-[1,2,4]oxadiazole-5-thione;

(1R-trans)3-(1-Aminomethyl-3-methyl-cyclopentylmethyl)-4H-[1,2,4]oxadiazole-5-thione;

(1R-cis)3-(1-Aminomethyl-3-methyl-cyclopentylmethyl)-4H-[1,2,4]oxadiazole-5-thione;

(1S-trans)3-(1-Aminomethyl-3-methyl-cyclopentylmethyl)-4H-[1,2,4]oxadiazole-5-thione;

(1α,3α,4α)3-(1-Aminomethyl-3,4-dimethyl-cyclopentylmethyl)-4H-[1,2,4]oxadiazole-5-thione;

(1α,3β,4β)3-(1-Aminomethyl-3,4-dimethyl-cyclopentylmethyl)-4H-[1,2,4]oxadiazole-5-thione;

(S)3-(1-Aminomethyl-3,3-dimethyl-cyclopentylmethyl)-4H-[1,2,4]oxadiazole-5-thione;

(R)3-(1-Aminomethyl-3,3-dimethyl-cyclopentylmethyl)-4H-[1,2,4]oxadiazole-5-thione;

3-(1-Aminomethyl-3,3-dimethyl-cyclobutylmethyl)-4H-[1,2,4]oxadiazole-5-thione;

C-[1-(1H-Tetrazol-5-ylmethyl)-cyclohexyl]-methylamine;

(1S-cis)C-[3-Methyl-1-(1H-tetrazol-5-ylmethyl)-cyclohexyl]-methylamine;

(trans)C-[3,4-Dimethyl-1-(1H-tetrazol-5-ylmethyl)-cyclopentyl]-methylamine;

(1S-cis)C-[3-Methyl-1-(1H-tetrazol-5-ylmethyl)-cyclopentyl]-methylamine;

(1R-trans)C-[3-Methyl-1-(1H-tetrazol-5-ylmethyl)-cyclopentyl]-methylamine;

(1R-cis)C-[3-Methyl-1-(1H-tetrazol-5-ylmethyl)-cyclopentyl]-methylamine;

(1S-trans)C-[3-Methyl-1-(1H-tetrazol-5-ylmethyl)-cyclopentyl]-methylamine;

(1α,3α,4α)C-[3,4-Dimethyl-1-(1H-tetrazol-5-ylmethyl)-cyclopentyl]-methylamine;

(1α,3β,4β)C-[3,4-Dimethyl-1-(1H-tetrazol-5-ylmethyl)-cyclopentyl]-methylamine;

(S)C-[3,3-Dimethyl-1(1H-tetrazol-5-ylmethyl)-cyclopentyl]-methylamine;

(R)C-[3,3-Dimethyl-1(1H-tetrazol-5-ylmethyl)-cyclopentyl]-methylamine;

C-[3,3-Dimethyl-1-(1H-tetrazol-5-ylmethyl)-cyclobutyl]-methylamine;

N-[2-(1-Aminomethyl-cyclohexyl)-ethyl]-C,C,C-trifluoro-methanesulfonamide;

(1S-cis)N-[2-(1-Aminomethyl-3-methyl-cyclohexyl)-ethyl]-C,C,C-trifluoro-methanesulfonamide;

(trans)N-[2-(1-Aminomethyl-3,4-dimethyl-cyclopentyl)-ethyl]-C,C,C-trifluoro-methanesulfonamide;

(1R-cis)N-[2-(1-Aminomethyl-3-methyl-cyclopentyl)-ethyl]-C,C,C-trifluoro-methanesulfonamide;

(1S-trans)N-[2-(1-Aminomethyl-3-methyl-cyclopentyl)-ethyl]-C,C,C-trifluoro-methanesulfonamide;

(1S-cis)N-[2-(1-Aminomethyl-3-methyl-cyclopentyl)-ethyl]-C,C,C-trifluoro-methanesulfonamide;

(1R-trans)N-[2-(1-Aminomethyl-3-methyl-cyclopentyl)-ethyl]-C,C,C-trifluoro-methanesulfonamide;

(1α,3α,4α)N-[2-(1-Aminomethyl-3,4-dimethyl-cyclopentyl)-ethyl]-C,C,C-trifluoro-methanesulfonamide;

(1α,3β,4β)N-[2-(1-Aminomethyl-3,4-dimethyl-cyclopentyl)-ethyl]-C,C,C-trifluoro-methanesulfonamide;

(S)N-[2-(1-Aminomethyl-3,3-dimethyl-cyclopentyl)-ethyl]-C,C,C-trifluoro-methanesulfonamide;

(R)N-[2-(1-Aminomethyl-3,3-dimethyl-cyclopentyl)-ethyl]-C,C,C-trifluoro-methanesulfonamide;

N-[2-(1-Aminomethyl-3,3-dimethyl-cyclobutyl)-ethyl]-C,C,C-trifluoro-methanesulfonamide;

3-(1-Aminomethyl-cyclohexylmethyl)-4H-[1,2,4]thiadiazol-5-one;

(1S-cis)3-(1-Aminomethyl-3-methyl-cyclohexylmethyl)-4H-[1,2,4]thiadiazol-5-one;

(trans)3-(1-Aminomethyl-3,4-dimethyl-cyclopentylmethyl)-4H-[1,2,4]thiadiazol-5-one;

(1R-cis)3-(1-Aminomethyl-3-methyl-cyclopentylmethyl)-4H-[1,2,4]thiadiazol-5-one;

(1S-trans)3-(1-Aminomethyl-3-methyl-cyclopentylmethyl)-4H-[1,2,4]thiadiazol-5-one;

(1S-cis)3-(1-Aminomethyl-3-methyl-cyclopentylmethyl)-4H-[1,2,4]thiadiazol-5-one;

(1R-trans)3-(1-Aminomethyl-3-methyl-cyclopentylmethyl)-4H-[1,2,4]thiadiazol-5-one;

(1α,3α,4α)3-(1-Aminomethyl-3,4-dimethyl-cyclopentylmethyl)-4H-[1,2,4]thiadiazol-5-one;

(1α,3β,4β)3-(1-Aminomethyl-3,4-dimethyl-cyclopentylmethyl)-4H-[1,2,4]thiadiazol-5-one;

(S)3-(1-Aminomethyl-3,3-dimethyl-cyclopentylmethyl)-4H-[1,2,4]thiadiazol-5-one;

(R)3-(1-Aminomethyl-3,3-dimethyl-cyclopentylmethyl)-4H-[1,2,4]thiadiazol-5-one;

3-(1-Aminomethyl-3,3-dimethyl-cyclobutylmethyl)-4H-[1,2,4]thiadiazol-5-one;

C-[1-(2-Oxo-2,3-dihydro-2×4-[1,2,3,5]oxathiadiazol-4-ylmethyl)-cyclohexyl]-methylamine;

(1S-cis)C-[3-Methyl-1-(2-oxo-2,3-dihydro-2λ⁴-[1,2,3,5]oxathiadiazol-4-ylmethyl)-cyclohexyl]-methylamine;

(trans)C-[3,4-Dimethyl-1-(2-oxo-2,3-dihydro-2λ⁴-[1,2,3,5]oxathiadiazol-4-ylmethyl)-cyclopentyl]-methyl amine;

(1S-cis)C-[3-Methyl-1-(2-oxo-2,3-dihydro-2λ⁴-[1,2,3,5]oxathiadiazol-4-ylmethyl)-cyclopentyl]-methylamine;

(1R-trans)C-[3-Methyl-1-(2-oxo-2,3-dihydro-2λ⁴-[1,2,3,5]oxathiadiazol-4-ylmethyl)-cyclopentyl]-methylamine;

(1R-cis)C-[3-Methyl-1-(2-oxo-2,3-dihydro-2λ⁴-[1,2,3,5]oxathiadiazol-4-ylmethyl)-cyclopentyl]-methylamine;

(1S-trans)C-[3-Methyl-1-(2-oxo-2,3-dihydro-2λ⁴-[1,2,3,5]oxathiadiazol-4-ylmethyl)-cyclopentyl]-methylamine;

(1α,3α,4α)C-[3,4-Dimethyl-1-(2-oxo-2,3-dihydro-2λ⁴-[1,2,3,5]oxathiadiazol-4-ylmethyl)-cyclopentyl]-methylamine;

(1α,3β,4β)C-[3,4-Dimethyl-1-(2-oxo-2,3-dihydro-2λ⁴-[1,2,3,5]oxathiadiazol-4-ylmethyl)-cyclopentyl]-methylamine;

(S)C-[3,3-Dimethyl-1-(2-oxo-2,3-dihydro-2λ⁴-[1,2,3,5]oxathiadiazol-4-ylmethyl)-cyclopentyl]-methylamine;

(R)C-[3,3-Dimethyl-1-(2-oxo-2,3-dihydro-2λ⁴-[1,2,3,5]oxathiadiazol-4-ylmethyl)-cyclopentyl]-methylamine;

C-[3,3-Dimethyl-1-(2-oxo-2,3-dihydro-2λ⁴-[1,2,3,5]oxathiadiazol-4-ylmethyl)-cyclobutyl]-methylamine;

(1-Aminomethyl-cyclohexyl)-methanesulfonamide;

(1R-trans)(1-Aminomethyl-3-methyl-cyclohexyl)-methanesulfonamide;

(trans)(1-Aminomethyl-3,4-dimethyl-cyclopentyl)-methanesulfonamide;

(1S-trans)(1-Aminomethyl-3-methyl-cyclopentyl)-methanesulfonamide;

(1R-cis)(1-Aminomethyl-3-methyl-cyclopentyl)-methanesulfonamide;

(1R-trans)(1-Aminomethyl-3-methyl-cyclopentyl)-methanesulfonamide;

(1S-cis)(1-Aminomethyl-3-methyl-cyclopentyl)-methanesulfonamide;

(1α,3β,4β)(1-Aminomethyl-3,4-dimethyl-cyclopentyl)-methanesulfonamide;

(1α,3α,4α)(1-Aminomethyl-3,4-dimethyl-cyclopentyl)-methanesulfonamide;

(R)(1-Aminomethyl-3,3-dimethyl-cyclopentyl)-methanesulfonamide;

(S)(1-Aminomethyl-3,3-dimethyl-cyclopentyl)-methanesulfonamide;

(1-Aminomethyl-3,3-dimethyl-cyclobutyl)-methanesulfonamide;

(1-Aminomethyl-cyclohexyl)-methanesulfonic acid;

(1R-trans) (1-Aminomethyl-3-methyl-cyclohexyl)-methanesulfonic acid;

(trans)(1-Aminomethyl-3,4-dimethyl-cyclopentyl)-methanesulfonic acid;

(1S-trans)(1-Aminomethyl-3-methyl-cyclopentyl)-methanesulfonic acid;

(1S-cis)(1-Aminomethyl-3-methyl-cyclopentyl)-methanesulfonic acid;

(1R-trans)(1-Aminomethyl-3-methyl-cyclopentyl)-methanesulfonic acid;

(1R-cis)(1-Aminomethyl-3-methyl-cyclopentyl)-methanesulfonic acid;

(1α,3β,4β,)(1-Aminomethyl-3,4-dimethyl-cyclopentyl)-methanesulfonic acid;

(1α,3α,4α)(1-Aminomethyl-3,4-dimethyl-cyclopentyl)-methanesulfonic acid;

(R)(1-Aminomethyl-3,3-dimethyl-cyclopentyl)-methanesulfonic acid;

(S)(1-Aminomethyl-3,3-dimethyl-cyclopentyl)-methanesulfonic acid;

(1-Aminomethyl-3,3-dimethyl-cyclobutyl)-methanesulfonic acid;

(1-Aminomethyl-cyclopentylmethyl)-phosphonic acid;

2-(1-Aminomethyl-cyclopentyl)-N-hydroxy-acetamide;

N-[2-(1-Aminomethyl-cyclopentyl)-ethyl]-methanesulfonamide;

3-(1-Aminomethyl-cyclopentylmethyl)-4H-[1,2,4]oxadiazol-5-one;

3-(1-Aminomethyl-cyclopentylmethyl)-4H-[1,2,4]oxadiazole-5-thione;

C-[1-(1H-Tetrazol-5-ylmethyl)-cyclopentyl]-methylamine;

N-[2-(1-Aminomethyl-cyclopentyl)-ethyl]-C,C,C-trifluoro-methanesulfonamide;

3-(1-Aminomethyl-cyclopentylmethyl)-4H-[1,2,4]thiadiazol-5-one;

C-[1-(2-Oxo-2,3-dihydro-2λ⁴-[1,2,3,5]oxathiadiazol-4-ylmethyl)-cyclopentyl]-methylamine;

(1-Aminomethyl-cyclopentyl)-methanesulfonamide;

(1-Aminomethyl-cyclopentyl)-methanesulfonic acid;

(9-Aminomethyl-bicyclo[3.3.1]non-9-ylmethyl)-phosphonic acid;

2-(9-Aminomethyl-bicyclo[3.3.1]non-9-yl)-N-hydroxy-acetamide;

N-[2-(9-Aminomethyl-bicyclo[3.3. I]non-9-yl)-ethyl]-methanesulfonamide;

3-(9-Aminomethyl-bicyclo[3.3.1]non-9-ylmethyl)-4H-[1,2,4]oxadiazol-5-one;

3-(9-Aminomethyl-bicyclo[3.3.1]non-9-ylmethyl)-4H-[1,2,4]oxadiazole-5-thione;

C-[9-(1H-Tetrazol-5-ylmethyl)-bicyclo[3.3.1]non-9-yl]-methylamine;

N-[2-(9-Aminomethyl-bicyclo[3.3.1]non-9-yl)-ethyl]-C,C,C-trifluoro-methanesulfonamide;

3-(9-Aminomethyl-bicyclo[3.3.1]non-9-ylmethyl)-4H-[1,2,4]thiadiazol-5-one;

C-[9-(2-Oxo-2,3-dihydro-2λ⁴-[1,2,3,5]oxathiadiazol-4-ylmethyl)-bicyclo[3.3.1]non-9-yl]-methyl amine;

(9-Aminomethyl-bicyclo[3.3.1]non-9-yl)-methanesulfonamide;

(9-Aminomethyl-bicyclo[3.3.1]non-9-yl)-methanesulfonic acid;

(2-Aminomethyl-adamantan-2-ylmethyl)-phosphonic acid;

2-(2-Aminomethyl-adamantan-2-yl)-N-hydroxy-acetamide;

N-[2-(2-Aminomethyl-adamantan-2-yl)-ethyl]-methanesulfonamide;

3-(2-Aminomethyl-adamantan-2-ylmethyl)-4H-[1,2,4]oxadiazol-5-one;

3-(2-Aminomethyl-adamantan-2-ylmethyl)-4H-[1,2,4]oxadiazole-5-thione;

C-[2-(1H-Tetrazol-5-ylmethyl)-adamantan-2-yl]-methylamine;

N-[2-(2-Aminomethyl-adamantan-2-yl)-ethyl]-C,C,C-trifluoro-methanesulfonamide;

3-(2-Aminomethyl-adamantan-2-ylmethyl)-4H-[1,2,4]thiadiazol-5-one;

C-[2-(2-Oxo-2,3-dihydro-2λ⁴-[1,2,3,5]oxathiadiazol-4-ylmethyl)-adamantan-2-yl]-methylamine;

(2-Aminomethyl-adamantan-2-yl)-methanesulfonamide;

(2-Aminomethyl-adamantan-2-yl)-methanesulfonic acid (1-Aminomethyl-cycloheptylmethyl)-phosphonic acid;

2-(1-Aminomethyl-cycloheptyl)-N-hydroxy-acetamide;

N-[2-(1-Aminomethyl-cycloheptyl)-ethyl]-methanesulfonamide;

3-(1-Aminomethyl-cycloheptylmethyl)-4H-[1,2,4]oxadiazole-5-thione;

N-[2-(1-Aminomethyl-cycloheptyl)-ethyl]-C,C,C-trifluoro-methanesulfonamide;

C-[1-(2-Oxo-2,3-dihydro-2,4-[1,2,3,5]oxathiadiazol-4-ylmethyl)-cycloheptyl]-methylamine;

20 (1-Aminomethyl-cycloheptyl)-methanesulfonamide; and

(1-Aminomethyl-cycloheptyl)-methanesulfonic acid, or a pharmaceutically acceptable salt thereof.

24. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formula IV

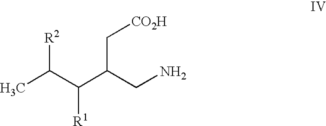

or a pharmaceutically acceptable salt thereof wherein:

R¹ is hydrogen, straight or branched alkyl of from 1 to 6 carbon atoms or phenyl;

R² is straight or branched alkyl of from 1 to 8 carbon atoms,

straight or branched alkenyl of from 2 to 8 carbon atoms,

cycloalkyl of from 3 to 7 carbon atoms,

alkoxy of from 1 to 6 carbon atoms,

-alkylcycloalkyl,

-alkylalkoxy,

-alkyl OH,

-alkylphenyl,

-alkylphenoxy,

-phenyl or substituted phenyl; and

R¹ is straight or branched alkyl of from 1 to 6 carbon atoms or phenyl when R² is methyl.

25. The combination according to Embodiment 24, wherein the Alpha-2-delta ligand is a compound of Formula IV, or a pharmaceutically acceptable salt thereof, wherein R¹ is hydrogen, and R² is alkyl.

26. The combination according to Embodiment 24, wherein the Alpha-2-delta ligand is a compound of Formula IV, or a pharmaceutically acceptable salt thereof, wherein R¹ is methyl, and R² is alkyl.

27. The combination according to Embodiment 24, wherein the Alpha-2-delta ligand is a compound of Formula IV, or a pharmaceutically acceptable salt thereof, wherein R¹ is methyl, and R² is methyl or ethyl.

28. The combination according to Embodiment 24, wherein the Alpha-2-delta ligand is a compound of Formula IV, or a pharmaceutically acceptable salt thereof, selected from:

3-Aminomethyl-5-methylheptanoic acid;

3-Aminomethyl-5-methyl-octanoic acid;

3-Aminomethyl-5-methyl-nonanoic acid;

3-Aminomethyl-5-methyl-decanoic acid;

3-Aminomethyl-5-methyl-undecanoic acid;

3-Aminomethyl-5-methyl-dodecanoic acid;

3-Aminomethyl-5-methyl-tridecanoic acid;

3-Aminomethyl-5-cyclopropyl-hexanoic acid;

3-Aminomethyl-5-cyclobutyl-hexanoic acid;

3-Aminomethyl-5-cyclopentyl-hexanoic acid;

3-Aminomethyl-5-cyclohexyl-hexanoic acid;

3-Aminomethyl-5-trifluoromethyl-hexanoic acid;

3-Aminomethyl-5-phenyl-hexanoic acid;

3-Aminomethyl-5-(2-chlorophenyl)-hexanoic acid;

3-Aminomethyl-5-(3-chlorophenyl)-hexanoic acid;

3-Aminomethyl-5-(4-chlorophenyl)-hexanoic acid;

3-Aminomethyl-5-(2-methoxyphenyl)-hexanoic acid;

3-Aminomethyl-5-(3-methoxyphenyl)-hexanoic acid;

3-Aminomethyl-5-(4-methoxyphenyl)-hexanoic acid; and

3-Aminomethyl-5-(phenylmethyl)-hexanoic acid, or a pharmaceutically acceptable salt thereof.

29. The combination according to Embodiment 24, wherein the Alpha-2-delta ligand is a compound of Formula IV, or a pharmaceutically acceptable salt thereof, selected from:

(3R,4S)3-Aminomethyl-4,5-dimethyl-hexanoic acid;

3-Aminomethyl-4,5-dimethyl-hexanoic acid;

(3R,4S)3-Aminomethyl-4,5-dimethyl-hexanoic acid MP;

(3S,4S)3-Aminomethyl-4,5-dimethyl-hexanoic acid;

(3R,4R)3-Aminomethyl-4,5-dimethyl-hexanoic acid MP;

3-Aminomethyl-4-isopropyl-hexanoic acid;

3-Aminomethyl-4-isopropyl-heptanoic acid;

3-Aminomethyl-4-isopropyl-octanoic acid;

3-Aminomethyl-4-isopropyl-nonanoic acid;

3-Aminomethyl-4-isopropyl-decanoic acid; and

3-Aminomethyl-4-phenyl-5-methyl-hexanoic acid, or a pharmaceutically acceptable salt thereof.

30. The combination according to Embodiment 24, wherein the Alpha-2-delta ligand is a compound of Formula IV, or a pharmaceutically acceptable salt thereof, selected from:

(3S,5R)-3-Aminomethyl-5-methyl-heptanoic acid, or a pharmaceutically acceptable salt thereof.

31. The combination according to Embodiment 24, wherein the Alpha-2-delta ligand is a compound of Formula IV, or a pharmaceutically acceptable salt thereof, selected from:

(3S,5R)-3-Aminomethyl-5-methyl-octanoic acid, or a pharmaceutically acceptable salt thereof.

32. The combination according to Embodiment 24, wherein the Alpha-2-delta ligand is a compound of Formula IV, or a pharmaceutically acceptable salt thereof, selected from:

(3S,5R)-3-Aminomethyl-5-methyl-nonanoic acid, or a pharmaceutically acceptable salt thereof.

33. The combination according to Embodiment 24, wherein the Alpha-2-delta ligand is a compound of Formula IV, or a pharmaceutically acceptable salt thereof, selected from:

(3S,5R)-3-Aminomethyl-5-methyl-decanoic acid, or a pharmaceutically acceptable salt thereof.

34. The combination according to Embodiment 24, wherein the Alpha-2-delta ligand is a compound of Formula IV, or a pharmaceutically acceptable salt thereof, selected from:

(3S,5R)-3-Aminomethyl-5-methyl-undecanoic acid, or a pharmaceutically acceptable salt thereof.

35. The combination according to Embodiment 24, wherein the Alpha-2-delta ligand is a compound of Formula IV, or a pharmaceutically acceptable salt thereof, selected from:

(3S,5R)-3-Aminomethyl-5-methyl-dodecanoic acid, or a pharmaceutically acceptable salt thereof.

36. The combination according to Embodiment 24, wherein the Alpha-2-delta ligand is a compound of Formula IV, or a pharmaceutically acceptable salt thereof, selected from:

(3S,5R)-3-Aminomethyl-5,9-dimethyl-decanoic acid;

(3S,5R)-3-Aminomethyl-5-methyl-heptanoic acid;

(3S,5R)-3-Aminomethyl-5,7-dimethyl-octanoic acid;

(3S,5R)-3-Aminomethyl-5,10-dimethyl-undecanoic acid;

(3S,5R)-3-Aminomethyl-5,8-dimethyl-nonanoic acid;

(3S,5R)-3-Aminomethyl-6-cyclopropyl-5-methyl-hexanoic acid;

(3S,5R)-3-Aminomethyl-6-cyclobutyl-5-methyl-hexanoic acid;

(3S,5R)-3-Aminomethyl-6-cyclopentyl-5-methyl-hexanoic acid;

(3S,5R)-3-Aminomethyl-6-cyclohexyl-5-methyl-hexanoic acid;

(3S,5R)-3-Aminomethyl-7-cyclopropyl-5-methyl-heptanoic acid;

(3S,5R)-3-Aminomethyl-7-cyclobutyl-5-methyl-heptanoic acid;

(3S,5R)-3-Aminomethyl-7-cyclopentyl-5-methyl-heptanoic acid;

(3S,5R)-3-Aminomethyl-7-cyclohexyl-5-methyl-heptanoic acid;

(3S,5R)-3-Aminomethyl-8-cyclopropyl-5-methyl-octanoic acid;

(3S,5R)-3-Aminomethyl-8-cyclobutyl-5-methyl-octanoic acid;

(3S,5R)-3-Aminomethyl-8-cyclopentyl-5-methyl-octanoic acid;

(3S,5R)-3-Aminomethyl-8-cyclohexyl-5-methyl-octanoic acid;

(3S,5S)-3-Aminomethyl-6-fluoro-5-methyl-hexanoic acid;

(3S,5S)-3-Aminomethyl-7-fluoro-5-methyl-heptanoic acid;

(3S,5R)-3-Aminomethyl-8-fluoro-5-methyl-octanoic acid;

(3S,5R)-3-Aminomethyl-9-fluoro-5-methyl-nonanoic acid;

(3S,5S)-3-Aminomethyl-7,7,7-trifluoro-5-methyl-heptanoic acid; and

(3S,5R)-3-Aminomethyl-8,8,8-trifluoro-5-methyl-octanoic acid, or a pharmaceutically acceptable salt thereof.

37. The combination according to Embodiment 24, wherein the Alpha-2-delta ligand is a compound of Formula IV, or a pharmaceutically acceptable salt thereof, selected from:

(3S,5S)-3-Aminomethyl-5-methoxy-hexanoic acid;

(3S,5R)-3-Aminomethyl-8-hydroxy-5-methyl-octanoic acid;

(3S,5S)-3-Aminomethyl-5-ethoxy-hexanoic acid;

(3S,5S)-3-Aminomethyl-5-propoxy-hexanoic acid;

(3S,5S)-3-Aminomethyl-5-isopropoxy-hexanoic acid;

(3S,5S)-3-Aminomethyl-5-tert-butoxy-hexanoic acid;

(3S,5S)-3-Aminomethyl-5-fluoromethoxy-hexanoic acid;

(3S,5S)-3-Aminomethyl-5-(2-fluoro-ethoxy)-hexanoic acid;

(3S,5S)-3-Aminomethyl-5-(3,3,3-trifluoro-propoxy)-hexanoic acid;

(3S,5S)-3-Aminomethyl-5-phenoxy-hexanoic acid;

(3S,5S)-3-Aminomethyl-5-(4-chloro-phenoxy)-hexanoic acid;

(3S,5S)-3-Aminomethyl-5-(3-chloro-phenoxy)-hexanoic acid;

(3S,5S)-3-Aminomethyl-5-(2-chloro-phenoxy)-hexanoic acid;

(3S,5S)-3-Aminomethyl-5-(4-fluoro-phenoxy)-hexanoic acid;

(3S,5S)-3-Aminomethyl-5-(3-fluoro-phenoxy)-hexanoic acid;

(3S,5S)-3-Aminomethyl-5-(2-fluoro-phenoxy)-hexanoic acid;

(3S,5S)-3-Aminomethyl-5-(4-methoxy-phenoxy)-hexanoic acid;

(3S,5S)-3-Aminomethyl-5-(3-methoxy-phenoxy)-hexanoic acid;

(3S,5S)-3-Aminomethyl-5-(2-methoxy-phenoxy)-hexanoic acid;

(3S,5S)-3-Aminomethyl-5-(4-nitro-phenoxy)-hexanoic acid;

(3S,5S)-3-Aminomethyl-5-(3-nitro-phenoxy)-hexanoic acid;

(3S,5S)-3-Aminomethyl-5-(2-nitro-phenoxy)-hexanoic acid;

(3S,5S)-3-Aminomethyl-6-hydroxy-5-methyl-hexanoic acid;

(3S,5S)-3-Aminomethyl-6-methoxy-5-methyl-hexanoic acid;

(3S,5S)-3-Aminomethyl-6-ethoxy-5-methyl-hexanoic acid;

(3S,5S)-3-Aminomethyl-5-methyl-6-propoxy-hexanoic acid;

(3S,5S)-3-Aminomethyl-6-isopropoxy-5-methyl-hexanoic acid;

(3S,5S)-3-Aminomethyl-6-tert-butoxy-5-methyl-hexanoic acid;

(3S,5S)-3-Aminomethyl-6-fluoromethoxy-5-methyl-hexanoic acid;

(3S,5S)-3-Aminomethyl-6-(2-fluoro-ethoxy)-5-methyl-hexanoic acid;

(3S,5S)-3-Aminomethyl-5-methyl-6-(3,3,3-trifluoro-propoxy)-hexanoic acid;

(3S,5S)-3-Aminomethyl-5-methyl-6-phenoxy-hexanoic acid;

(3S,5S)-3-Aminomethyl-6-(4-chloro-phenoxy)-5-methylhexanoic acid;

(3S,5S)-3-Aminomethyl-6-(3-chloro-phenoxy)-5-methylhexanoic acid;

(3S,5S)-3-Aminomethyl-6-(2-chloro-phenoxy)-5-methylhexanoic acid;

(3S,5S)-3-Aminomethyl-6-(4-fluoro-phenoxy)-5-methylhexanoic acid;

(3S,5S)-3-Aminomethyl-6-(3-fluoro-phenoxy)-5-methylhexanoic acid;

(3S,5S)-3-Aminomethyl-6-(2-fluoro-phenoxy)-5-methylhexanoic acid;

(3S,5S)-3-Aminomethyl-6-(4-methoxy-phenoxy)-5-methylhexanoic acid;

(3S,5S)-3-Aminomethyl-6-(3-methoxy-phenoxy)-5-methylhexanoic acid;

(3S,5S)-3-Aminomethyl-6-(2-methoxy-phenoxy)-5-methylhexanoic acid;

(3S,5S)-3-Aminomethyl-5-methyl 6-(4-trifluoromethyl-phenoxy)-hexanoic acid;

(3S,5S)-3-Aminomethyl-5-methyl 6-(3-trifluoromethyl-phenoxy)-hexanoic acid;

(3S,5S)-3-Aminomethyl-5-methyl 6-(2-trifluoromethyl-phenoxy)-hexanoic acid;

(3S,5S)-3-Aminomethyl-5-methyl 6-(4-nitro-phenoxy)-hexanoic acid;

(3S,5S)-3-Aminomethyl-5-methyl 6-(3-nitro-phenoxy)-hexanoic acid;

(3S,5S)-3-Aminomethyl-5-methyl 6-(2-nitro-phenoxy)-hexanoic acid;

(3S,5S)-3-Aminomethyl-6-benzyloxy-5-methyl-hexanoic acid;

(3S,5S)-3-Aminomethyl-7-hydroxy-5-methyl-heptanoic acid;

(3S,5S)-3-Aminomethyl-7-methoxy-5-methyl-heptanoic acid;

(3S,5S)-3-Aminomethyl-7-ethoxy-5-methyl-heptanoic acid;

(3S,5S)-3-Aminomethyl-5-methyl-7-propoxy-heptanoic acid;

(3S,5S)-3-Aminomethyl-7-isopropoxy-5-methyl-heptanoic acid;

(3S,5S)-3-Aminomethyl-7-tert-butoxy-5-methyl-heptanoic acid;

(3S,5S)-3-Aminomethyl-7-fluoromethoxy-5-methylheptanoic acid;

(3S,5S)-3-Aminomethyl-7-(2-fluoro-ethoxy)-5-methylheptanoic acid;

(3S,5S)-3-Aminomethyl-5-methyl-7-(3,3,3-trifluoro-propoxy)-heptanoic acid;

(3S,5S)-3-Aminomethyl-7-benzyloxy-5-methyl-heptanoic acid;

(3S,5S)-3-Aminomethyl-5-methyl-7-phenoxy-heptanoic acid;

(3S,5S)-3-Aminomethyl-7-(4-chloro-phenoxy)-5-methylheptanoic acid;

(3S,5S)-3-Aminomethyl-7-(3-chloro-phenoxy)-5-methylheptanoic acid;

(3S,5S)-3-Aminomethyl-7-(2-chloro-phenoxy)-5-methylheptanoic acid;

(3S,5S)-3-Aminomethyl-7-(4-fluoro-phenoxy)-5-methylheptanoic acid;

(3S,5S)-3-Aminomethyl-7-(3-fluoro-phenoxy)-5-methylheptanoic acid;

(3S,5S)-3-Aminomethyl-7-(2-fluoro-phenoxy)-5-methylheptanoic acid;

(3S,5S)-3-Aminomethyl-7-(4-methoxy-phenoxy)-5-methylheptanoic acid;

(3S,5S)-3-Aminomethyl-7-(3-methoxy-phenoxy)-5-methylheptanoic acid;

(3S,5S)-3-Aminomethyl-7-(2-methoxy-phenoxy)-5-methylheptanoic acid;

(3S,5S)-3-Aminomethyl-5-methyl-7-(4-trifluoromethyl-phenoxy)-heptanoic acid;

(3S,5S)-3-Aminomethyl-5-methyl-7-(3-trifluoromethyl-phenoxy)-heptanoic acid;

(3S,5S)-3-Aminomethyl-5-methyl-7-(2-trifluoromethyl-phenoxy)-heptanoic acid;

(3S,5S)-3-Aminomethyl-5-methyl-7-(4-nitro-phenoxy)-heptanoic acid;

(3S,5S)-3-Aminomethyl-5-methyl-7-(3-nitro-phenoxy)-heptanoic acid;

(3S,5S)-3-Aminomethyl-5-methyl-7-(2-nitro-phenoxy)-heptanoic acid;

(3S,5S)-3-Aminomethyl-5-methyl-6-phenyl-hexanoic acid;

(3S,5S)-3-Aminomethyl-6-(4-chloro-phenyl)-5-methylhexanoic acid;

(3S,5S)-3-Aminomethyl-6-(3-chloro-phenyl)-5-methylhexanoic acid;

(3S,5S)-3-Aminomethyl-6-(2-chloro-phenyl)-5-methylhexanoic acid;

(3S,5S)-3-Aminomethyl-6-(4-methoxy-phenyl)-5-methylhexanoic acid;

(3S,5S)-3-Aminomethyl-6-(3-methoxy-phenyl)-5-methylhexanoic acid;

(3S,5S)-3-Aminomethyl-6-(2-methoxy-phenyl)-5-methylhexanoic acid;

(3S,5S)-3-Aminomethyl-6-(4-fluoro-phenyl)-5-methylhexanoic acid;

(3S,5S)-3-Aminomethyl-6-(3-fluoro-phenyl)-5-methylhexanoic acid;

(3S,5S)-3-Aminomethyl-6-(2-fluoro-phenyl)-5-methylhexanoic acid;

(3S,5R)-3-Aminomethyl-5-methyl-7-phenyl-heptanoic acid;

(3S,5R)-3-Aminomethyl-7-(4-chloro-phenyl)-5-methylheptanoic acid;

(3S,5R)-3-Aminomethyl-7-(3-chloro-phenyl)-5-methylheptanoic acid;

(3S,5R)-3-Aminomethyl-7-(2-chloro-phenyl)-5-methylheptanoic acid;

(3S,5R)-3-Aminomethyl-7-(4-methoxy-phenyl)-5-methylheptanoic acid;

(3S,5R)-3-Aminomethyl-7-(3-methoxy-phenyl)-5-methylheptanoic acid;

(3S,5R)-3-Aminomethyl-7-(2-methoxy-phenyl)-5-methylheptanoic acid;

(3S,5R)-3-Aminomethyl-7-(4-fluoro-phenyl)-5-methylheptanoic acid;

(3S,5R)-3-Aminomethyl-7-(3-fluoro-phenyl)-5-methylheptanoic acid;

(3S,5R)-3-Aminomethyl-7-(2-fluoro-phenyl)-5-methylheptanoic acid;

(3S,5S)-3-Aminomethyl-5-methyl-hept-6-enoic acid;

(3S,5R)-3-Aminomethyl-5-methyl-oct-7-enoic acid;

(3S,5R)-3-Aminomethyl-5-methyl-non-8-enoic acid;

(E)-(3S,5S)-3-Aminomethyl-5-methyl-oct-6-enoic acid;

(Z)-(3S,5S)-3-Aminomethyl-5-methyl-oct-6-enoic acid;

(Z)-(3S,5S)-3-Aminomethyl-5-methyl-non-6-enoic acid;

(E)-(3S,5S)-3-Aminomethyl-5-methyl-non-6-enoic acid;

(E)-(3S,5R)-3-Aminomethyl-5-methyl-non-7-enoic acid;

(Z)-(3S,5R)-3-Aminomethyl-5-methyl-non-7-enoic acid;

(Z)-(3S,5R)-3-Aminomethyl-5-methyl-dec-7-enoic acid;

(E)-(3S,5R)-3-Aminomethyl-5-methyl-undec-7-enoic acid;

(3S,5S)-3-Aminomethyl-5,6,6-trimethyl-heptanoic acid;

(3S,5S)-3-Aminomethyl-5,6-dimethyl-heptanoic acid;

(3S,5S)-3-Aminomethyl-5-cyclopropyl-hexanoic acid;

(3S,5S)-3-Aminomethyl-5-cyclobutyl-hexanoic acid;

(3S,5S)-3-Aminomethyl-5-cyclopentyl-hexanoic acid;

(3S,5S)-3-Aminomethyl-5-cyclohexyl-hexanoic acid;

(3S,5R)-3-Aminomethyl-5-methyl-8-phenyl-octanoic acid;

(3S,5S)-3-Aminomethyl-5-methyl-6-phenyl-hexanoic acid;

(3S,5R)-3-Aminomethyl-5-methyl-7-phenyl-heptanoic acid;

(3R,4R,5R)-3-Aminomethyl-4,5-dimethyl-heptanoic acid; and

(3R,4R,5R)-3-Aminomethyl-4,5-dimethyl-octanoic acid, or a pharmaceutically acceptable salt thereof.

38. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formula (1A) or Formula (1B)

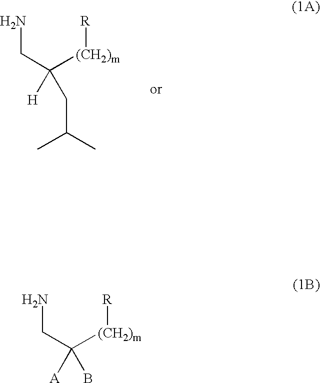

or a pharmaceutically acceptable salt thereof wherein:

n is an integer of from 0 to 2;

R is sulfonamide,

amide,

phosphonic acid,

heterocycle,

sulfonic acid, or

hydroxamic acid;

A is hydrogen or methyl; and

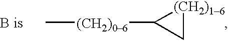

straight or branched alkyl of from 1 to 11 carbons, or —(CH₂)₁₋₄—Y—(CH₂)₀₋₄-phenyl wherein Y is —O—, —S—, —NR′₃ wherein

R₁₃ is alkyl of from 1 to 6 carbons, cycloalkyl of from 3 to 8 carbons, benzyl or phenyl wherein benzyl or phenyl can be unsubstituted or substituted with from 1 to 3 substituents each independently selected from alkyl, alkoxy, halogen, hydroxy, carboxy, carboalkoxy, trifluoromethyl, and nitro.

39. The combination according to Embodiment 38, wherein R is a sulfonamide selected from —NHSO₂R¹⁵ and —SO₂NHR¹⁵, wherein R¹⁵ is straight or branched alkyl or trifluoromethyl.

40. The combination according to Embodiment 38, wherein R is a phosphonic acid, —PO₃H₂.

41. The combination according to Embodiment 38, wherein R is

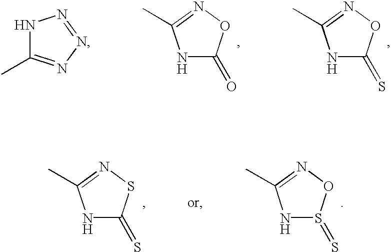

42. The combination according to Embodiment 38, wherein R is

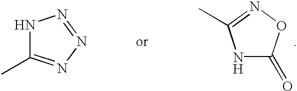

43. The combination according to Embodiment 38, wherein the compound of Formulas (1A) or (1B), or a pharmaceutically acceptable salt thereof, is selected from:

4-Methyl-2-(1H-tetrazol-5-ylmethyl)-pentylamine;

3-(2-Aminomethyl-4-methyl-pentyl)-4H-[1,2,4]oxadiazole-5-thione, HCl;

(2-Aminomethyl-4-methyl-pentyl)-phosphonic acid;

3-(3-Amino-2-cyclopentyl-propyl)-4H-[1,2,4]oxadiazol-5-one;

3-(3-Amino-2-cyclopentyl-propyl)-4H-[1,2,4]thiadiazol-5-one;

2-Cyclopentyl-3-(2-oxo-2,3-dihydro-2λ⁴-[1,2,3,5]oxathiadiazol-4-yl)-propylamine;

3-(3-Amino-2-cyclobutyl-propyl)-4H-[1,2,4]oxadiazol-5-one;

3-(3-Amino-2-cyclobutyl-propyl)-4H-[1,2,4]thiadiazol-5-one; and

2-Cyclobutyl-3-(2-oxo-2,3-dihydro-2λ⁴-[1,2,3,5]oxathiadiazol-4-yl)-propylamine, or a pharmaceutically acceptable salt thereof.

44. The combination according to Embodiment 38, wherein the compound of Formulas (1A) or (1B), or a pharmaceutically acceptable salt thereof, is named 3-(2-aminomethyl-4-methyl-pentyl)-4H-[1,2,4]oxadiazol-5-one, or a pharmaceutically acceptable salt thereof.

45. The combination according to Embodiment 38, wherein the compound of Formulas (1A) or (1B), or a pharmaceutically acceptable salt thereof, is named 3-(2-aminomethyl-4-methyl-pentyl)-4H-[1,2,4]-oxadiazol-5-one hydrochloride.

46. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formulas V, VI, VII, or VIII

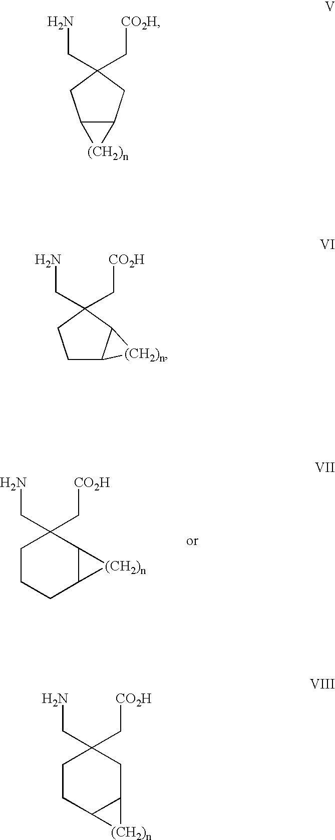

or pharmaceutically acceptable salt thereof,

wherein n is an integer of from 1 to 4, and

where there are stereocenters, each center may be independently R or S.

47. The combination according to Embodiment 46, wherein n is an integer of from 2 to 4.

48. The combination according to Embodiment 46, wherein the Alpha-2-delta ligand is a compound of Formula V, or a pharmaceutically acceptable salt thereof.

49. The combination according to Embodiment 46, wherein the Alpha-2-delta ligand is a compound of Formula V, VI, VII, or VIII, or a pharmaceutically acceptable salt thereof, selected from:

(1α,6α,8β)(2-Aminomethy-octahydro-inden-2-yl)-acetic acid (2-Aminomethyl-octahydro-inden-2-yl)-acetic acid; (2-Aminomethyl-octahydro-pentalen-2-yl)-acetic acid; (2-Aminomethyl-octahydro-pentalen-2-yl)-acetic acid; (3-Aminomethyl-bicyclo[3.2.0)hept-3-yl)-acetic acid; (3-Aminomethyl-bicyclo[3.2.0]hept-3-yl)-acetic acid; and

(2-Aminomethyl-octahydro-inden-2-yl)-acetic acid, or a pharmaceutically acceptable salt thereof.

50. The combination according to Embodiment 46, wherein the Alpha-2-delta ligand is a compound of Formula V, VI, VII, or VIII, or a pharmaceutically acceptable salt thereof, selected from:

(1α,5β)(3-Aminomethyl-bicyclo[3.1.0]hex-3-yl)-acetic acid,

(1α,5β)(3-Aminomethyl-bicyclo[3.2.0]hept-3-yl)-acetic acid,

(1α,5β)(2-Aminomethyl-octahydro-pentalen-2-yl)-acetic acid,

(1α,6β)(2-Aminomethyl-octahydro-inden-2-yl)-acetic acid,

(1α,7β)(2-Aminomethyl-decahydro-azulen-2-yl)-acetic acid,

(1α,5β)(3-Aminomethyl-bicyclo[3.1.0]hex-3-yl)-acetic acid,

(1α,5β)3-Aminomethyl-bicyclo[3.2.0]hept-3-yl)-acetic acid,

(1α,5β)(2-Aminomethyl-octahydro-pentalen-2-yl)-acetic acid,

(1α,6β)(2-Aminomethyl-octahydro-inden-2-yl)-acetic acid,

(1α,7β)(2-Aminomethyl-decahydro-azulen-2-yl)-acetic acid,

(1α,3α,5α)(3-Aminomethyl-bicyclo[3.1.0]hex-3-yl)-acetic acid,

(1α,3α,5α)(2-Aminomethyl-octahydro-pentalen-2-yl)-acetic acid,

(1α,6α,8α)(2-Aminomethyl-octahydro-inden-2-yl)-acetic acid,

(1α,7α,9α)(2-Aminomethyl-decahydro-azulen-2-yl)-acetic acid,

(1α,3β,5α)(3-Aminomethyl-bicyclo[3.1.0]hex-3-yl)-acetic acid,

(1α,3β,5α)(3-Aminomethyl-bicyclo[3.2.0]hept-3-yl)-acetic acid,

(1α,3β,5α)(2-Aminomethyl-octahydro-pentalen-2-yl)-acetic acid,

(1α,6α,8β)(2-Aminomethyl-octahydro-inden-2-yl)-acetic acid,

(1α,7α,9β)(2-Aminomethyl-decahydro-azulen-2-yl)-acetic acid,

((1R,3R,6R)-3-Aminomethyl-bicyclo[4.1.0]hept-3-yl)-acetic acid,

((1R,3S,6R)-3-Aminomethyl-bicyclo[4.1.0]hept-3-yl)-acetic acid,

((1S,3S,6S)-3-Aminomethyl-bicyclo[4.1.0]hept-3-yl)-acetic acid,

((1S,3R,6S)-3-Aminomethyl-bicyclo[4.1.0]oct-3-yl)-acetic acid,

((1R,3R,6S)-3-Aminomethyl-bicyclo[4.2.0]oct-3-yl)-acetic acid,

((1R,3S,6S)-3-Aminomethyl-bicyclo[4.2.0]oct-3-yl)-acetic acid,

((1S,3S,6R)-3-Aminomethyl-bicyclo[4.2.0]oct-3-yl)-acetic acid,

((1S,3R,6R)-3-Aminomethyl-bicyclo[4.2.0]oct-3-yl)-acetic acid,

((3αR,5R,7αS)-5-Aminomethyl-octahydro-inden-5-yl)-acetic acid,

((3αR,5S,7αS)-5-Aminomethyl-octahydro-inden-5-yl)-acetic acid,

((3αS,5S,7αR)-5-Aminomethyl-octahydro-inden-5-yl)-acetic acid,

((3αS,5R,7αR)-5-Aminomethyl-octahydro-inden-5-yl)-acetic acid,

((2R,4αS,8αR)-2-Aminomethyl-decahydro-naphthalen-2-yl)-acetic acid,

((2S,4αS,8αR)-2-Aminomethyl-decahydro-naphthalen-2-yl)-acetic acid,

((2S,4αR,8αS)-2-Aminomethyl-decahydro-naphthalen-2-yl)-acetic acid,

((2R,4αR,8αS)-2-Aminomethyl-decahydro-naphthalen-2-yl)-acetic acid,

((2R,4αS,9αR)-2-Aminomethyl-decahydro-benzocyclophepten-2-yl)-acetic acid,

((2S,4αS,9αR)-2-Aminomethyl-decahydro-benzocyclophepten-2-yl) acetic acid,

((2S,4αR,9αS)-2-Aminomethyl-decahydro-benzocyclophepten-2-yl) acetic acid,

((2R,4αR,9αS)-2-Aminomethyl-decahydro-benzocyclophepten-2-yl) acetic acid,

((1R,3R,6S)-3-Aminomethyl-bicyclo[4.1.0]hept-3-yl)-acetic acid,

((1R,3S,6S)-3-Aminomethyl-bicyclo[4.1.0]hept-3-yl)-acetic acid,

((1S,3S,6R)-3-Aminomethyl-bicyclo[4.1.0]hept-3-yl)-acetic acid,

((1S,3R,6R)-3-Aminomethyl-bicyclo[4.1.0]hept-3-yl)-acetic acid,

((1R,3R,6R)-3-Aminomethyl-bicyclo[4.2.0]oct-3-yl)-acetic acid,

((1R,3S,6R)-3-Aminomethyl-bicyclo[4.2.0]oct-3-yl)-acetic acid,

((1S,3S,6S)-3-Aminomethyl-bicyclo[4.2.0]oct-3-yl)-acetic acid,

((1S,3R,6S)-3-Aminomethyl-bicyclo[4.2.0]oct-3-yl)-acetic acid,

((3αR,5R,7αR)-5-Aminomethyl-octahydro-inden-5-yl)-acetic acid,

((3αR,5S,7αR)-5-Aminomethyl-octahydro-inden-5-yl)-acetic acid,

((3αS,5S,7αS)-5-Aminomethyl-octahydro-inden-5-yl)-acetic acid,

((3αS,5R,7αS)-5-Aminomethyl-octahydro-inden-5-yl)-acetic acid,

((2R,4αR,8αR)-2-Aminomethyl-decahydro-naphthalen-2-yl)-acetic acid,

((2S,4αS,8αR)-2-Aminomethyl-decahydro-naphthalen-2-yl)-acetic acid,

((2S,4αR,8αS)-2-Aminomethyl-decahydro-naphthalen-2-yl)-acetic acid,

((2R,4αS,8αS)-2-Aminomethyl-decahydro-naphthalen-2-yl)-acetic acid,

((2R,4αR,9αR)-2-Aminomethyl-decahydro-benzocyclophepten-2-yl)-acetic acid,

((2S,4αR,9αR)-2-Aminomethyl-decahydro-benzocyclophepten-2-yl)-acetic acid,

((2S,4αS,9αS)-2-Aminomethyl-decahydro-benzocyclophepten-2-yl)-acetic acid, and

((2R,4αS,9αS)-2-Aminomethyl-decahydro-benzocyclophepten-2-yl)-acetic acid, or a pharmaceutically acceptable salt thereof.

51. The combination according to Embodiment 46, wherein the Alpha-2-delta ligand is a compound of Formulas V, VI, VII, or VIII, or a pharmaceutically acceptable salt thereof, named (1α,3α,5α)(3-aminomethyl-bicyclo[3.2.0]hept-3-yl)-acetic acid, or a pharmaceutically acceptable salt thereof.

52. The combination according to Embodiment 46, wherein the Alpha-2-delta ligand is a compound of Formulas V, VI, VII, or VIII, or a pharmaceutically acceptable salt thereof, named (1α,3α,5α) (3-aminomethyl-bicyclo[3.2.0.]hept-3-yl)-acetic acid hydrochloride.

53. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formulas (1D) or (1E)

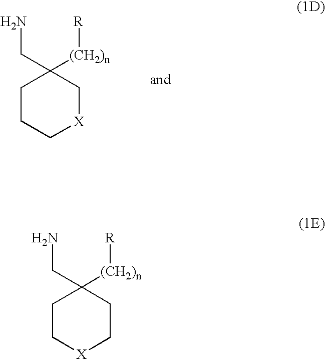

or a pharmaceutically acceptable salt thereof wherein:

n is an integer of from 0 to 2;

R is sulfonamide,

amide,

phosphonic acid,

heterocycle,

sulfonic acid, or

hydroxamic acid; and

X is —O—, —S—, —S(O)—, —S(O)₂—, or NR′₁ wherein R′₁ is hydrogen, straight or branched alkyl of from 1 to 6 carbons, benzyl, —C(O)R′₂ wherein R′₂ is straight or branched alkyl of 1 to 6 carbons, benzyl or phenyl or —CO₂R′₃ wherein R′₃ is straight or branched alkyl of from 1 to 6 carbons, or benzyl wherein the benzyl or phenyl groups can be unsubstituted or substituted by from 1 to 3 substituents selected from halogen, trifluoromethyl, and nitro.

54. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formula

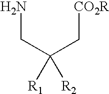

or a pharmaceutically acceptable salt thereof wherein:

R is hydrogen or lower alkyl;

R₁ is hydrogen or lower alkyl;

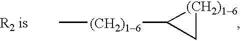

straight or branched alkyl of from 7 to 11 carbon atoms, or —(CH₂)₍₁₋₄₎—X—(CH₂)₍₀₋₄₎-phenyl wherein

X is —O—, —S—, —NR₃₋ wherein

R₃ is alkyl of from 1 to 6 carbons, cycloalkyl of from 3 to 8 carbons, benzyl or phenyl;

wherein phenyl and benzyl can be unsubstituted or substituted with from 1 to 3 substituents each independently selected from alkyl, alkoxy, halogen, hydroxy, carboxy, carboalkoxy, trifluoromethyl, amino, and nitro.

55. A combination, comprising valdecoxib, or a pharmaceutically acceptable salt thereof, and an Alpha-2-delta ligand, or a pharmaceutically acceptable salt thereof, wherein the Alpha-2-delta ligand is a compound of Formulas (1), (2), (3), (4), (5), (6), (7), or (8)

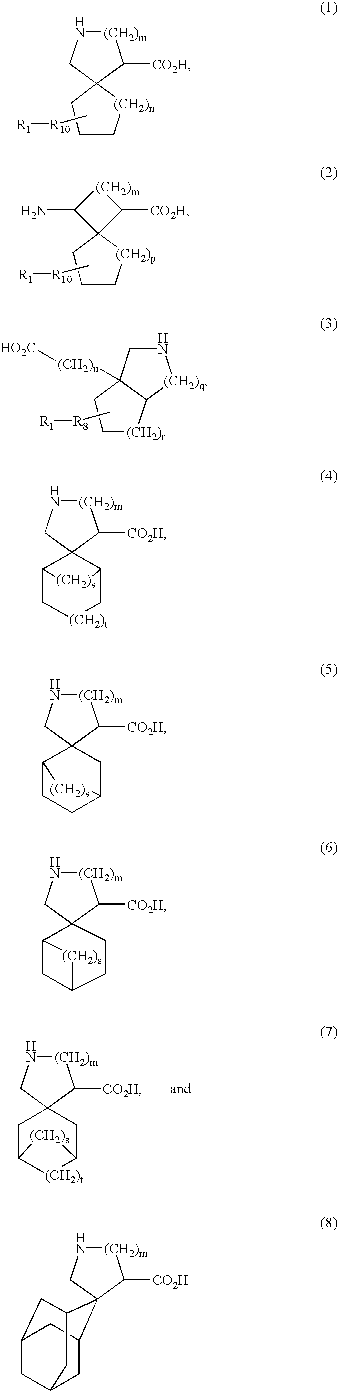

or a pharmaceutically acceptable salt thereof or a prodrug thereof wherein:

R₁ to R₁₀ are each independently selected from hydrogen or a straight or branched alkyl of from 1 to 6 carbons, benzyl, or phenyl;

m is an integer of from 0 to 3;

n is an integer of from 1 to 2;

p is an integer of from 1 to 2;

q is an integer of from 0 to 2;

r is an integer of from 1 to 2;

c is an integer of from 1 to 3;

t is an integer of from o to 2; and

u is an integer of from o to 1.

56. A combination comprising valdecoxib and a compound of Formula (9) or (9A)

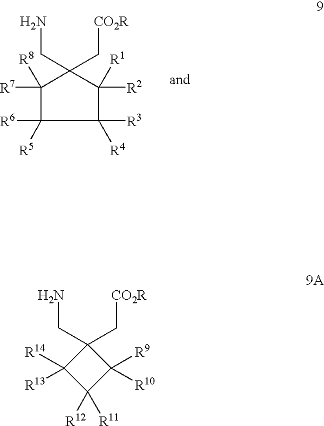

or a pharmaceutically acceptable salt thereof wherein:

R is hydrogen or a lower alkyl;

R¹ to R¹⁴ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen.

57. The combination according to Embodiment 56, wherein R¹ to R¹⁴ are selected from hydrogen, methyl, ethyl, propyl, isopropyl, butyl straight or branched, phenyl, or benzyl.

58. The combination according to Embodiment 56, wherein R¹ to R¹⁴ are selected from hydrogen, methyl, ethyl, or benzyl.

59. The combination according to Embodiment 56, wherein the compound of Formulas (9) or (9A) is named:

(3S,4S)-(1-Aminomethyl-3,4-dimethyl-cyclopentyl)-acetic acid;

or a pharmaceutically acceptable salt thereof.

60. The combination according to Embodiment 56, wherein the compound of Formulas (9) or (9A) is named:

(3S,4S)-(1-Aminomethyl-3,4-dimethyl-cyclopentyl)-acetic acid.

61. The combination according to Embodiment 56, wherein the compound of Formulas (9) or (9A) is selected from:

(1α,3α,4α)-(1-Aminomethyl-3,4-dimethyl-cyclopentyl)-acetic acid;

(1α,3α,4α)-(1-Aminomethyl-3,4-diethyl-cyclopentyl)-acetic acid;

(1α,3α,4α)-(1-Aminomethyl-3,4-diisopropyl-cyclopentyl)-acetic acid;

[1S-(1α,3α,4α)]-(1-Aminomethyl-3-ethyl-4-methyl-cyclopentyl)-acetic acid;

[1R-(1α,3α,4α)]-(1-Aminomethyl-3-ethyl-4-methyl-cyclopentyl)-acetic acid;

[1S-(1α,3α,4α)]-(1-Aminomethyl-3-isopropyl-4-methyl-cyclopentyl)-acetic acid;

[1R-(1α,3α,4α)]-(1-Aminomethyl-3-isopropyl-4-methyl-cyclopentyl)-acetic acid;

[1S-(1α,3α,4α)]-(1-Aminomethyl-3-ethyl-4-isopropyl-cyclopentyl)-acetic acid;

[1R-(1α,3α,4α)]-(1-Aminomethyl-3-ethyl-4-isopropyl-cyclopentyl)-acetic acid;

[1S-(1α,3α,4α)]-(1-Aminomethyl-3-tert-butyl-4-methyl-cyclopentyl)-acetic acid;

[1R-(1α,3α,4α)]-(1-Aminomethyl-3-tert-butyl-4-methyl-cyclopentyl)-acetic acid;

[1S-(1α,3α,4α)]-(1-Aminomethyl-3-tert-butyl-4-ethyl-cyclopentyl)-acetic acid;

[1R-(α,3α,4α)]-(1-Aminomethyl-3-tert-butyl-4-ethyl-cyclopentyl)-acetic acid;

[1S-(1α,3α,4α)]-(1-Aminomethyl-3-tert-butyl-4-isopropyl-cyclopentyl)-acetic acid;

[1R-(1α,3α,4α)]-(1-Aminomethyl-3-tert-butyl-4-isopropyl-cyclopentyl)-acetic acid;

(1α,3α,4α)-(1-Aminomethyl-3,4-di-tert-butyl-cyclopentyl)-acetic acid;

[1S-(1α,3α,4α)]-(1-Aminomethyl-3-methyl-4-phenyl-cyclopentyl)-acetic acid;

[1R-(1α,3α,4α)]-(1-Aminomethyl-3-methyl-4-phenyl-cyclopentyl)-acetic acid;

[1S-(1α,3α,4α)]-(1-Aminomethyl-3-benzyl-4-methyl-cyclopentyl)-acetic acid;

[1R-(1α,3α,4α)]-(1-Aminomethyl-3-benzyl-4-methyl-cyclopentyl)-acetic acid;

(1S-cis)-(1-Aminomethyl-3-methyl-cyclopentyl)-acetic acid;

(1S-cis)-(1-Aminomethyl-3-ethyl-cyclopentyl)-acetic acid;

(1S-cis)-(1-Aminomethyl-3-isopropyl-cyclopentyl)-acetic acid;

(1S-cis)-(1-Aminomethyl-3-tert-butyl-cyclopentyl)-acetic acid;

(1S-cis)-(1-Aminomethyl-3-phenyl-cyclopentyl)-acetic acid;

(1S-cis)-(1-Aminomethyl-3-benzyl-cyclopentyl)-acetic acid;

(1R-cis)-(1-Aminomethyl-3-methyl-cyclopentyl)-acetic acid;

(1R-cis)-(1-Aminomethyl-3-ethyl-cyclopentyl)-acetic acid;

(1R-cis)-(1-Aminomethyl-3-isopropyl-cyclopentyl)-acetic acid;

(1R-cis)-(1-Aminomethyl-3-tert-butyl-cyclopentyl)-acetic acid;

(1R-cis)-(1-Aminomethyl-3-phenyl-cyclopentyl)-acetic acid;

(1R-cis)-(1-Aminomethyl-3-benzyl-cyclopentyl)-acetic acid;

(S)-(1-Aminomethyl-3,3-dimethyl-cyclopentyl)-acetic acid;

(S)-(1-Aminomethyl-3,3-diethyl-cyclopentyl)-acetic acid;

(1-Aminomethyl-3,3,4,4-tetramethyl-cyclopentyl)-acetic acid;

(1-Aminomethyl-3,3,4,4-tetraethyl-cyclopentyl)-acetic acid;

(1α,3β,4β)-(1-Aminomethyl-3,4-dimethyl-cyclopentyl)-acetic acid;

(1α,3β,4β)-(1-Aminomethyl-3,4-diethyl-cyclopentyl)-acetic acid;

(1α,3β,4β)-(1-Aminomethyl-3,4-diisopropyl-cyclopentyl)-acetic acid;

[1R-(1α,3β,4β)]-(1-Aminomethyl-3-ethyl-4-methyl-cyclopentyl)-acetic acid;

[1S-(1α,3β,4β)]-(1-Aminomethyl-3-ethyl-4-methyl-cyclopentyl)-acetic acid;

[1R-(1α,3β,4β)]-(1-Aminomethyl-3-isopropyl-4-methyl-cyclopentyl)-acetic acid;

[1S-(1α,3β,4β)]-(1-Aminomethyl-3-isopropyl-4-methyl-cyclopentyl)-acetic acid;

[1R-(1α,3β,4β)]-(1-Aminomethyl-3-ethyl-4-isopropyl-cyclopentyl)-acetic acid;

[1S-(1α,3β,4β)]-(1-Aminomethyl-3-ethyl-4-isopropyl-cyclopentyl)-acetic acid;

[1R-(1α,3β,4β)]-(1-Aminomethyl-3-tert-butyl-4-methyl-cyclopentyl)-acetic acid;

[1S-(1α,3β,4β)]-(1-Aminomethyl-3-tert-butyl-4-methyl-cyclopentyl)-acetic acid;

[1R-(1α,3β,4β)]-(1-Aminomethyl-3-tert-butyl-4-ethyl-cyclopentyl)-acetic acid;

[1S-(1α,3β,4β)]-(1-Aminomethyl-3-tert-butyl-4-ethyl-cyclopentyl)-acetic acid;

[1R-(1α,3β,4β)]-(1-Aminomethyl-3-tert-butyl-4-isopropyl-cyclopentyl)-acetic acid;

[1S-(1α,3β,4β)]-(1-Aminomethyl-3-tert-butyl-4-isopropyl-cyclopentyl)-acetic acid;

(1α,3β,4β)-(1-Aminomethyl-3,4-di-tert-butyl-cyclopentyl)-acetic acid;

[1R-(1α,3β,4β)]-(1-Aminomethyl-3-methyl-4-phenyl-cyclopentyl)-acetic acid;

[1S-(1α,3β,4β)]-(1-Aminomethyl-3-methyl-4-phenyl-cyclopentyl)-acetic acid;

[1R-(1α,3β,4β)]-(1-Aminomethyl-3-benzyl-4-methyl-cyclopentyl)-acetic acid;

[1S-(1α,3β,4β)]-(1-Aminomethyl-3-benzyl-4-methyl-cyclopentyl)-acetic acid;

(1R-trans)-(1-Aminomethyl-3-methyl-cyclopentyl)-acetic acid;

(1R-trans)-(1-Aminomethyl-3-ethyl-cyclopentyl)-acetic acid;

(1R-trans)-(1-Aminomethyl-3-isopropyl-cyclopentyl)-acetic acid;

(1R-trans)-(1-Aminomethyl-3-tert-butyl-cyclopentyl)-acetic acid;

(1R-trans)-(1-Aminomethyl-3-phenyl-cyclopentyl)-acetic acid;

(1R-trans)-(1-Aminomethyl-3-benzyl-cyclopentyl)-acetic acid;

(1S-trans)-(1-Aminomethyl-3-methyl-cyclopentyl)-acetic acid;

(1S-trans)-(1-Aminomethyl-3-ethyl-cyclopentyl)-acetic acid;

(1S-trans)-(1-Aminomethyl-3-isopropyl-cyclopentyl)-acetic acid;

(1S-trans)-(1-Aminomethyl-3-tert-butyl-cyclopentyl)-acetic acid;

(1S-trans)-(1-Aminomethyl-3-phenyl-cyclopentyl)-acetic acid; and

(1S-trans)-(1-Aminomethyl-3-benzyl-cyclopentyl)-acetic acid.

(R)-(1-Aminomethyl-3,3-dimethyl-cyclopentyl)-acetic acid;

(R)-(1-Aminomethyl-3,3-diethyl-cyclopentyl)-acetic acid;

cis-(1-Aminomethyl-3-methyl-cyclobutyl)-acetic acid;

cis-(1-Aminomethyl-3-ethyl-cyclobutyl)-acetic acid;

cis-(1-Aminomethyl-3-isopropyl-cyclobutyl)-acetic acid;

cis-(1-Aminomethyl-3-tert-butyl-cyclobutyl)-acetic acid;

cis-(1-Aminomethyl-3-phenyl-cyclobutyl)-acetic acid;

cis-(1-Aminomethyl-3-benzyl-cyclobutyl)-acetic acid;

trans-(1-Aminomethyl-3-methyl-cyclobutyl)-acetic acid;

trans-(1-Aminomethyl-3-ethyl-cyclobutyl)-acetic acid;

trans-(1-Aminomethyl-3-isopropyl-cyclobutyl)-acetic acid;

trans-(1-Aminomethyl-3-tert-butyl-cyclobutyl)-acetic acid;

trans-(1-Aminomethyl-3-phenyl-cyclobutyl)-acetic acid;

trans-(1-Aminomethyl-3-benzyl-cyclobutyl)-acetic acid;

cis-(1-Aminomethyl-3-ethyl-3-methyl-cyclobutyl)-acetic acid;

cis-(1-Aminomethyl-3-isopropyl-3-methyl-cyclobutyl)-acetic acid;

cis-(1-Aminomethyl-3-tert-butyl-3-methyl-cyclobutyl)-acetic acid;

cis-(1-Aminomethyl-3-methyl-3-phenyl-cyclobutyl)-acetic acid;

cis-(1-Aminomethyl-3-benzyl-3-methyl-cyclobutyl)-acetic acid;

trans-(1-Aminomethyl-3-ethyl-3-methyl-cyclobutyl)-acetic acid;

trans-(1-Aminomethyl-3-isopropyl-3-methyl-cyclobutyl)-acetic acid;

trans-(1-Aminomethyl-3-tert-butyl-3-methyl-cyclobutyl)-acetic acid;

trans-(1-Aminomethyl-3-methyl-3-phenyl-cyclobutyl)-acetic acid;

trans-(1-Aminomethyl-3-benzyl-3-methyl-cyclobutyl)-acetic acid;

cis-(1-Aminomethyl-3-ethyl-3-isopropyl-cyclobutyl)-acetic acid;

cis-(1-Aminomethyl-3-tert-butyl-3-ethyl-cyclobutyl)-acetic acid;

cis-(1-Aminomethyl-3-ethyl-3-phenyl-cyclobutyl)-acetic acid;

cis-(1-Aminomethyl-3-benzyl-3-ethyl-cyclobutyl)-acetic acid;

trans-(1-Aminomethyl-3-ethyl-3-isopropyl-cyclobutyl)-acetic acid;

trans-(1-Aminomethyl-3-tert-butyl-3-ethyl-cyclobutyl)-acetic acid;

trans-(1-Aminomethyl-3-ethyl-3-phenyl-cyclobutyl)-acetic acid;

trans-(1-Aminomethyl-3-benzyl-3-ethyl-cyclobutyl)-acetic acid;

cis-(1-Aminomethyl-3-tert-butyl-3-isopropyl-cyclobutyl)-acetic acid;

cis-(1-Aminomethyl-3-isopropyl-3-phenyl-cyclobutyl)-acetic acid;

trans-(1-Aminomethyl-3-benzyl-3-isopropyl-cyclobutyl)-acetic acid;

cis-(1-Aminomethyl-3-tert-butyl-3-phenyl-cyclobutyl)-acetic acid;

trans-(1-Aminomethyl-3-benzyl-3-tert-butyl-cyclobutyl)-acetic acid;

trans-(1-Aminomethyl-3-tert-butyl-3-isopropyl-cyclobutyl)-acetic acid;

trans-(1-Aminomethyl-3-isopropyl-3-phenyl-cyclobutyl)-acetic acid;

cis-(1-Aminomethyl-3-benzyl-3-isopropyl-cyclobutyl)-acetic acid;

trans-(1-Aminomethyl-3-tert-butyl-3-phenyl-cyclobutyl)-acetic acid;

cis-(1-Aminomethyl-3-benzyl-3-tert-butyl-cyclobutyl)-acetic acid;

(1-Aminomethyl-3,3-dimethyl-cyclobutyl)-acetic acid;

(1-Aminomethyl-3,3-diethyl-cyclobutyl)-acetic acid;

(1-Aminomethyl-3,3-diisopropyl-cyclobutyl)-acetic acid;

(1-Aminomethyl-3,3-di-tert-butyl-cyclobutyl)-acetic acid;

(1-Aminomethyl-3,3-diphenyl-cyclobutyl)-acetic acid;

(1-Aminomethyl-3,3-dibenzyl-cyclobutyl)-acetic acid;

(1-Aminomethyl-2,2,4,4-tetramethyl-cyclobutyl)-acetic acid;

(1-Aminomethyl-2,2,3,3,4,4-hexamethyl-cyclobutyl)-acetic acid;

(R)-(1-Aminomethyl-2,2-dimethyl-cyclobutyl)-acetic acid;

(S)-(1-Aminomethyl-2,2-dimethyl-cyclobutyl)-acetic acid;

(1R-cis)-(1-Aminomethyl-2-methyl-cyclobutyl)-acetic acid;

[1R-(1α,2α,3α)]-(1-Aminomethyl-2,3-dimethyl-cyclobutyl)-acetic acid;

(1α,2α,4α)-(1-Aminomethyl-2,4-dimethyl-cyclobutyl)-acetic acid;

[1R-(1α,2α,3β)]-(1-Aminomethyl-2,3-dimethyl-cyclobutyl)-acetic acid;

(1α,2α,4β)-(1-Aminomethyl-2,4-dimethyl-cyclobutyl)-acetic acid;

(1S-trans)-(1-Aminomethyl-2-methyl-cyclobutyl)-acetic acid;

[1S-(1α,2β,3β)]-(1-Aminomethyl-2,3-dimethyl-cyclobutyl)-acetic acid;

(1α,2β,4β)-(1-Aminomethyl-2,4-dimethyl-cyclobutyl)-acetic acid;

[1S-(1α,2β,3α)]-(1-Aminomethyl-2,3-dimethyl-cyclobutyl)-acetic acid;

(1α,2β,4α)-(1-Aminomethyl-2,4-dimethyl-cyclobutyl)-acetic acid;

(1R-trans)-(1-Aminomethyl-2-methyl-cyclobutyl)-acetic acid;

[1R-(1α,2β,3β)]-(1-Aminomethyl-2,3-dimethyl-cyclobutyl)-acetic acid;

[1R-(1α,2β,4β)]-(1-Aminomethyl-2-ethyl-4-methyl-cyclobutyl)-acetic acid;

[1R-(1α,2β,3α)]-(1-Aminomethyl-2,3-dimethyl-cyclobutyl)-acetic acid;

(1α,2β,4α)-(1-Aminomethyl-2,4-dimethyl-cyclobutyl)-acetic acid;

(1S-cis)-(1-Aminomethyl-2-methyl-cyclobutyl)-acetic acid;

[1S-(1α,2α,3α)]-(1-Aminomethyl-2,3-dimethyl-cyclobutyl)-acetic acid;

[1S-(1α,2α,3α)]-(1-Aminomethyl-2,4-dimethyl-cyclobutyl)-acetic acid;

[1S-(1α,2β,3α)]-(1-Aminomethyl-2,3-dimethyl-cyclobutyl)-acetic acid;

(1α,2α,4β)-(1-Aminomethyl-2,4-dimethyl-cyclobutyl)-acetic acid;

(3R,4R)-(1-Aminomethyl-3,4-dimethyl-cyclopentyl)-acetic acid;

(3S,4S))-(1-Aminomethyl-3,4-diethyl-cyclopentyl)-acetic acid;

(3R,4R)-(1-Aminomethyl-3,4-diethyl-cyclopentyl)-acetic acid;

(3S,4S)-(1-Aminomethyl-3,4-diisopropyl-cyclopentyl)-acetic acid;

(3R,4R)-(1-Aminomethyl-3,4-diisopropyl-cyclopentyl)-acetic acid;

(3S,4S)-(1-Aminomethyl-3,4-di-tert-butyl-cyclopentyl)-acetic acid;

(3R,4R)-(1-Aminomethyl-3,4-di-tert-butyl-cyclopentyl)-acetic acid;

(3S,4S)-(1-Aminomethyl-3,4-diphenyl-cyclopentyl)-acetic acid;

(3R,4R)-(1-Aminomethyl-3,4-diphenyl-cyclopentyl)-acetic acid;

(3S,4S)-(1-Aminomethyl-3,4-dibenzyl-cyclopentyl)-acetic acid;

(3R,4R)-(1-Aminomethyl-3,4-dibenzyl-cyclopentyl)-acetic acid;

[1S-(1α,3α,4β)]-(1-Aminomethyl-3-methyl-4-ethyl-cyclopentyl)-acetic acid;

[1R-(1α,3β,4α)]-(1-Aminomethyl-3-methyl-4-ethyl-cyclopentyl)-acetic acid;

[1R-(1α,3α,4β)]-(1-Aminomethyl-3-methyl-4-ethyl-cyclopentyl)-acetic acid;

[1S-(1α,3β,4α)]-(1-Aminomethyl-3-methyl-4-ethyl-cyclopentyl)-acetic acid;

[1S-(1α,3α,4β)]-(1-Aminomethyl-3-methyl-4-isopropyl-cyclopentyl)-acetic acid;

[1R-(1α,3β,4α)]-(1-Aminomethyl-3-methyl-4-isopropyl-cyclopentyl)-acetic acid;

[1R-(1α,3α,4β)]-(1-Aminomethyl-3-methyl-4-isopropyl-cyclopentyl)-acetic acid;

[1S-(1α,3β,4α)]-(1-Aminomethyl-3-methyl-4-isopropyl-cyclopentyl)-acetic acid;

[1S-(1α,3α,4α)]-(1-Aminomethyl-3-methyl-4-tert-butyl-cyclopentyl)-acetic acid;

[1R-(1α,3β,4α)]-(1-Aminomethyl-3-methyl-4-tert-butyl-cyclopentyl)-acetic acid;

[1R-(1α,3α,4β)]-(1-Aminomethyl-3-methyl-4-tert-butyl-cyclopentyl)-acetic acid;

[1S-(1α,3β,4β)]-(1-Aminomethyl-3—methyl-4-tert-butyl-cyclopentyl)-acetic acid;

[1S-(1α,3α,4β)]-(1-Aminomethyl-3-methyl-4-phenyl-cyclopentyl)-acetic acid;

[1R-(1α,3β,4α)]J-(1-Aminomethyl-3-methyl-4-phenyl-cyclopentyl)-acetic acid;

[1R-(1α,3α,4β)]-(1-Aminomethyl-3-methyl-4-phenyl-cyclopentyl)-acetic acid;

[1S-(1α,3β,4α)]-Aminomethyl-3-methyl-4-phenyl-cyclopentyl)-acetic acid;

[1S-(1α,3α,4β)]-(1-Aminomethyl-3-benzyl-4-methyl-cyclopentyl)-acetic acid;

[1R-(1α,3β,4α)]-(1-Aminomethyl-3-benzyl-4-methyl-cyclopentyl)-acetic acid;

[1R-(1α,3α,4β)]-(1-Aminomethyl-3-benzyl-4-methyl-cyclopentyl)-acetic acid;

[1S-1α,3β,4α)]-(1-Aminomethyl-3-benzyl-4-methyl-cyclopentyl)-acetic acid;

[1S-(1α,3α,4β)]-(1-Aminomethyl-3-ethyl-4-isopropyl-cyclopentyl)-acetic acid;

[1R-(1α,3β,4α)]-(1-Aminomethyl-3-ethyl-4-isopropyl-cyclopentyl)-acetic acid;

[1R-(1α,3α,4β)]-(1-Aminomethyl-3-ethyl-4-isopropyl-cyclopentyl)-acetic acid;

[1S-(1α,3β,4α)]-(1-Aminomethyl-3-ethyl-4-isopropyl-cyclopentyl)-acetic acid;

[1S-(1α,3α,4β)]-(1-Aminomethyl-3-tert-butyl-4-ethyl-cyclopentyl)-acetic acid;

[1R-(1α,3β,4α)]-(1-Aminomethyl-3-tert-butyl-4-ethyl-cyclopentyl)-acetic acid;

[1R-(1α,3α,4β)]-(1-Aminomethyl-3-tert-butyl-4-ethyl-cyclopentyl)-acetic acid;

[1S-(1α,3β,4α)]-(1-Aminomethyl-3-tert-butyl-4-ethyl-cyclopentyl)-acetic acid;

[1S-(1α,3α,4β)]-(1-Aminomethyl-3-ethyl-4-phenyl-cyclopentyl)-acetic acid;

[1R-(1α,3β,4α)]-(1-Aminomethyl-3-ethyl-4-phenyl-cyclopentyl)-acetic acid;

[1R-(1α,3α,4β)]-(1-Aminomethyl-3-ethyl-4-phenyl-cyclopentyl)-acetic acid;

[1S-(1α,3β,4α)]-(1-Aminomethyl-3-ethyl-4-phenyl-cyclopentyl)-acetic acid;

[1S-(1α,3α,4β)]-(1-Aminomethyl-3-benzyl-4-ethyl-cyclopentyl)-acetic acid;

[1R-(1α,3β,4α)]-(1-Aminomethyl-3-benzyl-4-ethyl-cyclopentyl)-acetic acid;

[1R-(1α,3α,4β)]-(1-Aminomethyl-3-benzyl-4-ethyl-cyclopentyl)-acetic acid;

[1S-(1α,3β,4α)]-(1-Aminomethyl-3-benzyl-4-ethyl-cyclopentyl)-acetic acid;

[1S-(1α,3α,4β)]-(1-Aminomethyl-3-tert-butyl-4-isopropyl-cyclopentyl)-acetic acid;

[1R-(1α,3β,4α)]-(1-Aminomethyl-3-tert-butyl-4-isopropyl-cyclopentyl)-acetic acid;

[1R-(1α,3α,4β)]-(1-Aminomethyl-3-tert-butyl-4-isopropyl-cyclopentyl)-acetic acid;

[1S-(1α,3β,4α)]-(1-Aminomethyl-3-tert-butyl-4-isopropyl-cyclopentyl)-acetic acid;

[1S-(1α,3α,4β)]-(1-Aminomethyl-3-isopropyl-4-phenyl-cyclopentyl)-acetic acid;

[1R-(1α,3β,4α)]-(1-Aminomethyl-3-isopropyl-4-phenyl-cyclopentyl)-acetic acid;

[1R-(1α,3α,4β)]-(1-Aminomethyl-3-isopropyl-4-phenyl-cyclopentyl)-acetic acid;

[1S-(1α,3β,4α)]-(1-Aminomethyl-3-isopropyl-4-phenyl-cyclopentyl)-acetic acid;

[1S-(1α,3α,4β)]-(1-Aminomethyl-3-benzyl-4-isopropyl-cyclopentyl)-acetic acid;

[1R-(1α,3β,4α)]-(1-Aminomethyl-3-benzyl-4-isopropyl-cyclopentyl)-acetic acid;

[1R-(1α,3α,4β)]-(1-Aminomethyl-3-benzyl-4-isopropyl-cyclopentyl)-acetic acid;

[1S-(1α,3β,4α)]-(1-Aminomethyl-3-benzyl-4-isopropyl-cyclopentyl)-acetic acid;

[1S-(1α,3α,4β)]-(1-Aminomethyl-3-tert-butyl-4-phenyl-cyclopentyl)-acetic acid;

[1R-(1α,3β,4α)]-(1-Aminomethyl-3-tert-butyl-4-phenyl-cyclopentyl)-acetic acid;

[1R-(1α,3α,4β)]-(1-Aminomethyl-3-tert-butyl-4-phenyl-cyclopentyl)-acetic acid;

[1S-(1α,3β,4α)]-(1-Aminomethyl-3-tert-butyl-4-phenyl-cyclopentyl)-acetic acid;

[1R-(1α,3α,4β)]-(1-Aminomethyl-3-benzyl-4-tert-butyl-cyclopentyl)-acetic acid;

[1S-(1α,3β,4α)]-(1-Aminomethyl-3-benzyl-4-tert-butyl-cyclopentyl)-acetic acid;

[1S-(1α,3α,4β)]-(1-Aminomethyl-3-benzyl-4-tert-butyl-cyclopentyl)-acetic acid;

[1R-(1α,3β,4α)]-(1-Aminomethyl-3-benzyl-4-tert-butyl-cyclopentyl)-acetic acid;

[1S-(1α,3α,4β)]-(1-Aminomethyl-3-benzyl-4-phenyl-cyclopentyl)-acetic acid;

[1R-(1α,3β,4α)]-(1-Aminomethyl-3-benzyl-4-phenyl-cyclopentyl)-acetic acid;

[1R-(1α,3α,4β)]-(1-Aminomethyl-3-benzyl-4-phenyl-cyclopentyl)-acetic acid;

[1S-(1α,3β,4α)]-(1-Aminomethyl-3-benzyl-4-phenyl-cyclopentyl)-acetic acid;

(1R-cis)-(1-Aminomethyl-2-methyl-cyclopentyl)-acetic acid;

(1S-cis)-(1-Aminomethyl-2-methyl-cyclopentyl)-acetic acid;

(1R-trans)-(1-Aminomethyl-2-methyl-cyclopentyl)-acetic acid;

(1S-trans)-(1-Aminomethyl-2-methyl-cyclopentyl)-acetic acid;

(R)-(1-Aminomethyl-2,2-dimethyl-cyclopentyl)-acetic acid;

(S)-(1-Aminomethyl-2,2-dimethyl-cyclopentyl)-acetic acid;

(1-Aminomethyl-2,2,5,5-tetramethyl-cyclopentyl)-acetic acid;

(1α,2β,5β)-(1-Aminomethyl-2,5-dimethyl-cyclopentyl)-acetic acid;

(2R,5R)-(1-Aminomethyl-2,5-dimethyl-cyclopentyl)-acetic acid;

(2S.5S)-(1-Aminomethyl-2,5-dimethyl-cyclopentyl)-acetic acid;

(1α,2α,5α)-(1-Aminomethyl-2,5-dimethyl-cyclopentyl)-acetic acid;

[1R-(1α,2α,3α)]-(1-Aminomethyl-2,3-dimethyl-cyclopentyl)-acetic acid;

[1R-(1α,2β,3α)]-(1-Aminomethyl-2,3-dimethyl-cyclopentyl)-acetic acid;

[1R-(1α,2α,3β)]-(1-Aminomethyl-2,3-dimethyl-cyclopentyl)-acetic acid;

[1R-(1α,2β,3β)]-(1-Aminomethyl-2,3-dimethyl-cyclopentyl)-acetic acid;

[1S-(1α,2α,3α)]-(1-Aminomethyl-2,3-dimethyl-cyclopentyl)-acetic acid;

[1S-(1α,2β,3α)]-(1-Aminomethyl-2,3-dimethyl-cyclopentyl)-acetic acid;

[1S-(1α,2α,3β)]-(1-Aminomethyl-2,3-dimethyl-cyclopentyl)-acetic acid;

[1S-(1α,2β,3β)]-(1-Aminomethyl-2,3-dimethyl-cyclopentyl)-acetic acid;

[1R-(1α,2α,4α)]-(1-Aminomethyl-2,4-dimethyl-cyclopentyl)-acetic acid;

[1S-(1α,2α,4α)]-(1-Aminomethyl-2,4-dimethyl-cyclopentyl)-acetic acid;

[1R-(1α,2α,4β)]-(1-Aminomethyl-2,4-dimethyl-cyclopentyl)-acetic acid;

[1S-(1α,2α,4β)]-(1-Aminomethyl-2,4-dimethyl-cyclopentyl)-acetic acid;

[1R-(1α,2β,4α)]-(1-Aminomethyl-2,4-dimethyl-cyclopentyl)-acetic acid;

[1S-(1α,2β,4α)]-(1-Aminomethyl-2,4-dimethyl-cyclopentyl)-acetic acid;

[1R-(1α,2β,4β)]-(1-Aminomethyl-2,4-dimethyl-cyclopentyl)-acetic acid; and

[1S-(1α,2β,4β)]-(1-Aminomethyl-2,4-dimethyl-cyclopentyl)-acetic acid;

or a pharmaceutically acceptable salt thereof.

62. A pharmaceutical composition, comprising a combination of valdecoxib, or a pharmaceutically acceptable salt thereof, and an Alpha-2-delta ligand, or a pharmaceutically acceptable salt thereof, that is not a compound of Formulas

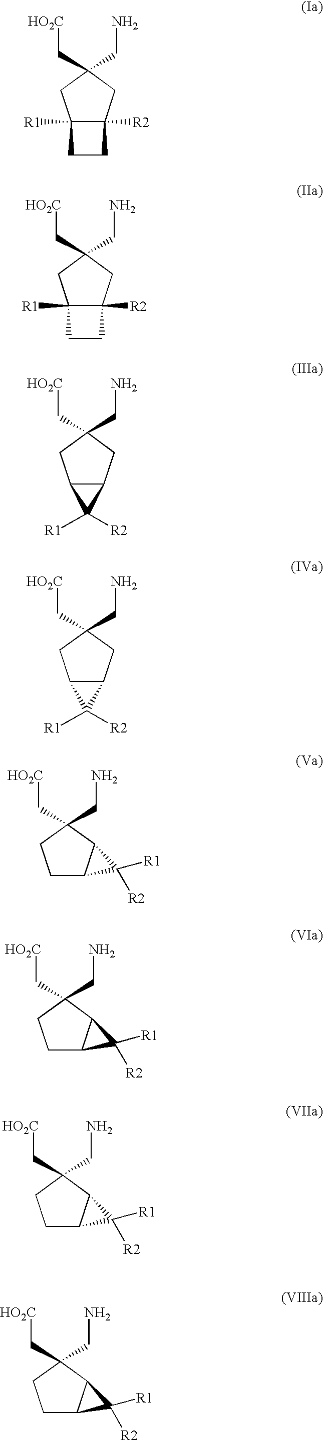

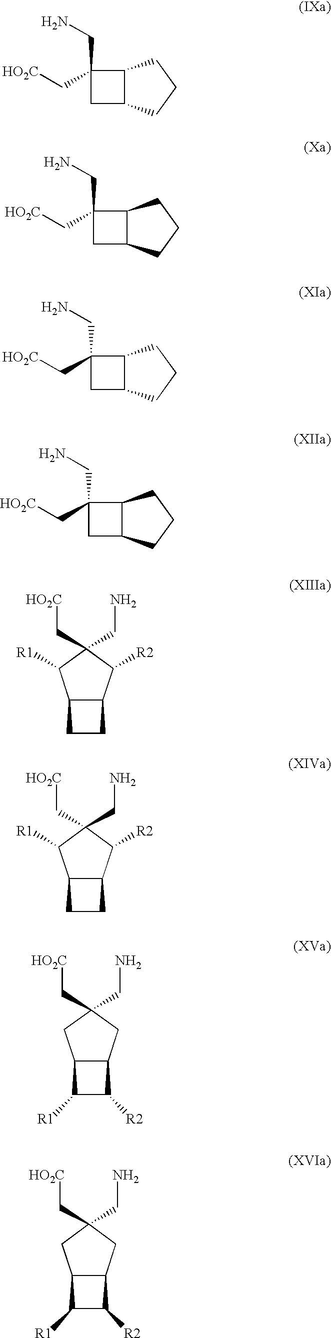

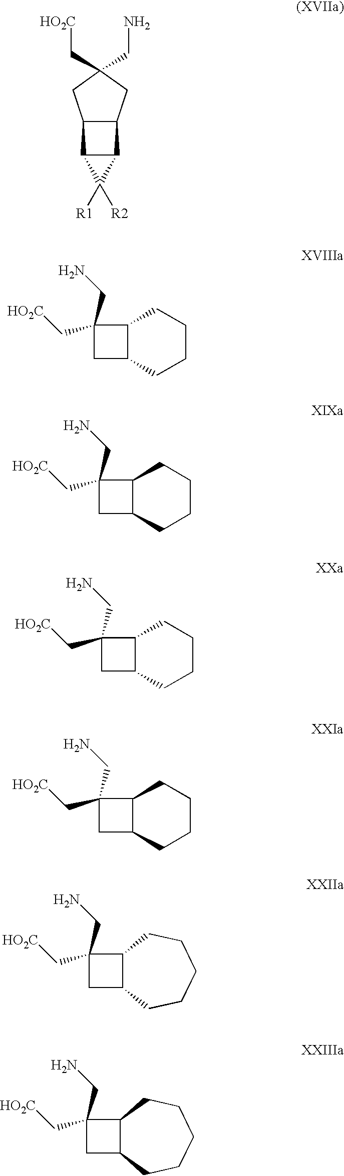

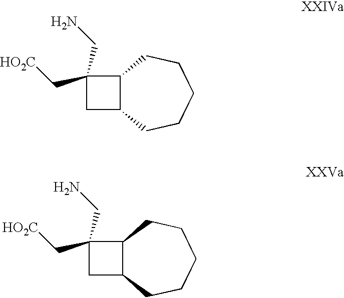

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa), and a pharmaceutically acceptable carrier, diluent, or excipient.

63. The pharmaceutical composition according to Embodiment 62, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

64. The pharmaceutical composition according to Embodiment 62, wherein the Alpha-2-delta ligand, or a pharmaceutically acceptable salt thereof, is a compound named 3-(1-aminomethyl-cyclohexylmethyl)-4H-[1,2,4]oxadiazol-5-one hydrochloride.

65. The pharmaceutical composition according to Embodiment 62, wherein the Alpha-2-delta ligand is a compound named gabapentin.

66. The pharmaceutical composition according to Embodiment 62, wherein the Alpha-2-delta ligand is a compound which is a pharmaceutically acceptable salt of gabapentin.

67. The pharmaceutical composition according to Embodiment 62, wherein the Alpha-2-delta ligand is a compound named pregabalin.

68. The pharmaceutical composition according to Embodiment 62, wherein the Alpha-2-delta ligand is a compound which is a pharmaceutically acceptable salt of pregabalin.

69. The pharmaceutical composition according to Embodiment 62, wherein the Alpha-2-delta ligand, or a pharmaceutically acceptable salt thereof, is a compound named (3S,4S)-(1-Aminomethyl-3,4-dimethylcyclopentyl)-acetic acid, or a pharmaceutically acceptable salt thereof.

70. The pharmaceutical composition according to Embodiment 62, wherein the Alpha-2-delta ligand, or a pharmaceutically acceptable salt thereof, is a compound named (3S,4S)-(1-Aminomethyl-3,4-dimethylcyclopentyl)-acetic acid.

71. A method of treating cartilage damage in a mammal in need thereof, comprising administering to the mammal a therapeutically effective amount of a combination, comprising valdecoxib, or a pharmaceutically acceptable salt thereof, and an Alpha-2-delta ligand, or a pharmaceutically acceptable salt thereof, that is not a compound of Formulas

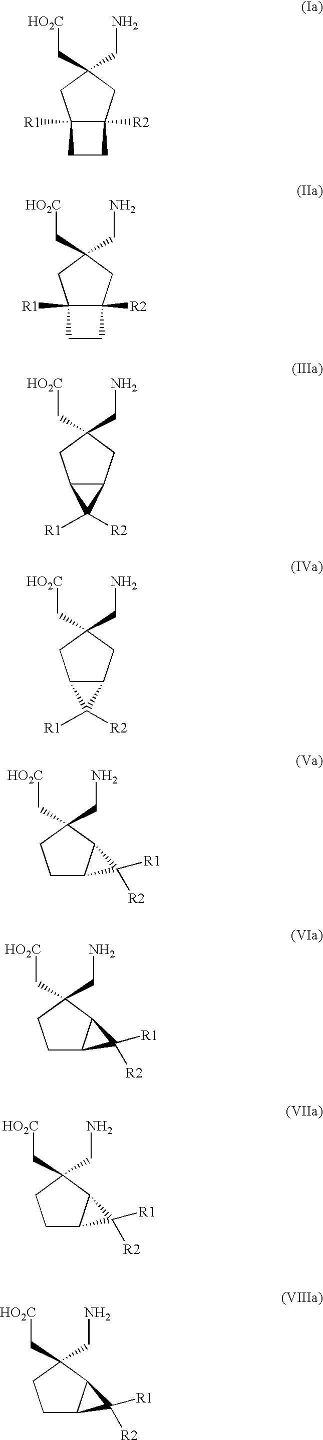

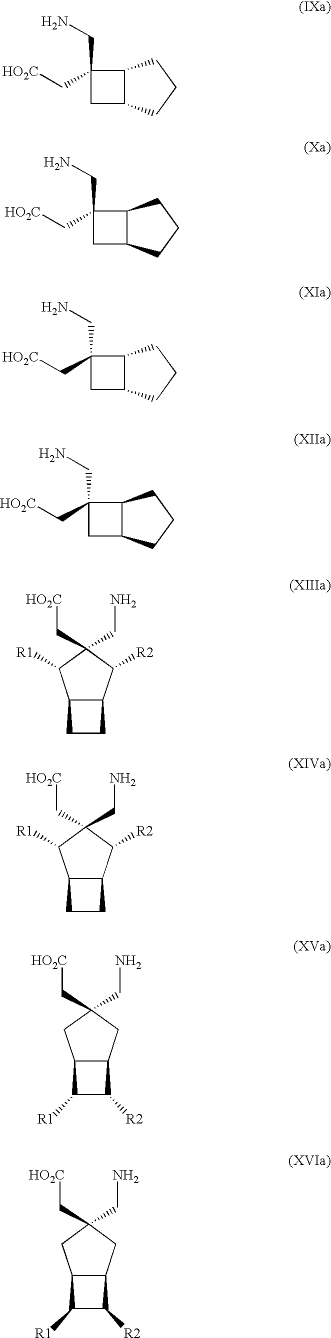

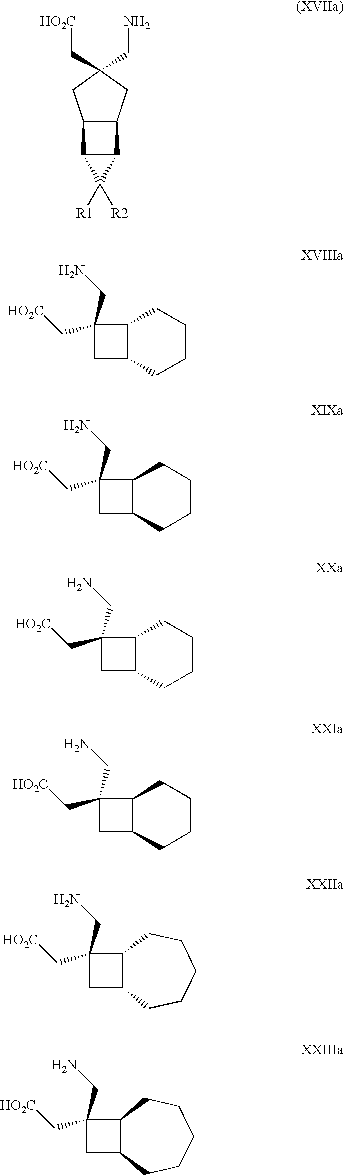

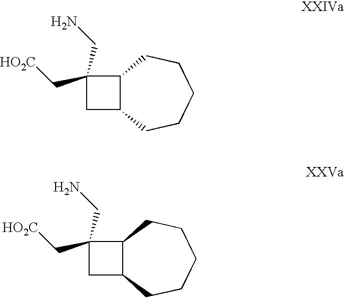

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

72. The method according to Embodiment 71, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

73. The method according to Embodiment 71, wherein the Alpha-2-delta ligand, or a pharmaceutically acceptable salt thereof, is a compound named 3-(1-aminomethyl-cyclohexylmethyl)-4H-[1,2,4]oxadiazol-5-one hydrochloride.

74. The method according to Embodiment 71, wherein the Alpha-2-delta ligand is a compound named gabapentin.

75. The method according to Embodiment 71, wherein the Alpha-2-delta ligand is a compound which is a pharmaceutically acceptable salt of gabapentin.

76. The method according to Embodiment 71, wherein the Alpha-2-delta ligand is a compound named pregabalin.

77. The method according to Embodiment 71, wherein the Alpha-2-delta ligand is a compound which is a pharmaceutically acceptable salt of pregabalin.

78. The method according to Embodiment 71, wherein the Alpha-2-delta ligand, or a pharmaceutically acceptable salt thereof, is a compound named (3S,4S)-(1-Aminomethyl-3,4-dimethyl-cyclopentyl)-acetic acid, or a pharmaceutically acceptable salt thereof

79. The method according to Embodiment 71, wherein the Alpha-2-delta ligand, or a pharmaceutically acceptable salt thereof, is a compound named (3S,4S)-(1-Aminomethyl-3,4-dimethyl-cyclopentyl)-acetic acid.

79A. The method according to any one of Embodiments 71 to 79, wherein the mammal having cartilage damage does not have joint pain.

79B. The method according to any one of Embodiments 71 to 79, wherein the mammal having cartilage damage has joint pain.

80. A method of treating inflammation in a mammal in need thereof, comprising administering to the mammal a therapeutically effective amount of a combination, comprising valdecoxib, or a pharmaceutically acceptable salt thereof, and an Alpha-2-delta ligand, or a pharmaceutically acceptable salt thereof, that is not a compound of Formulas

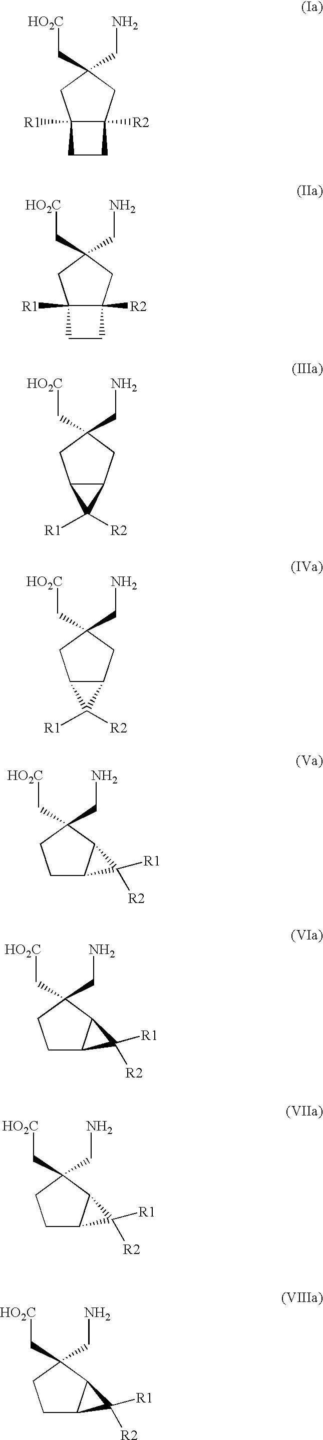

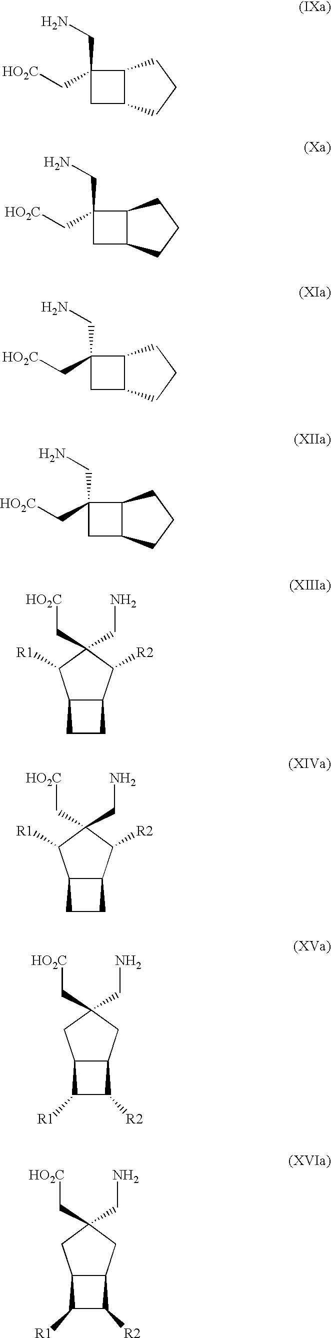

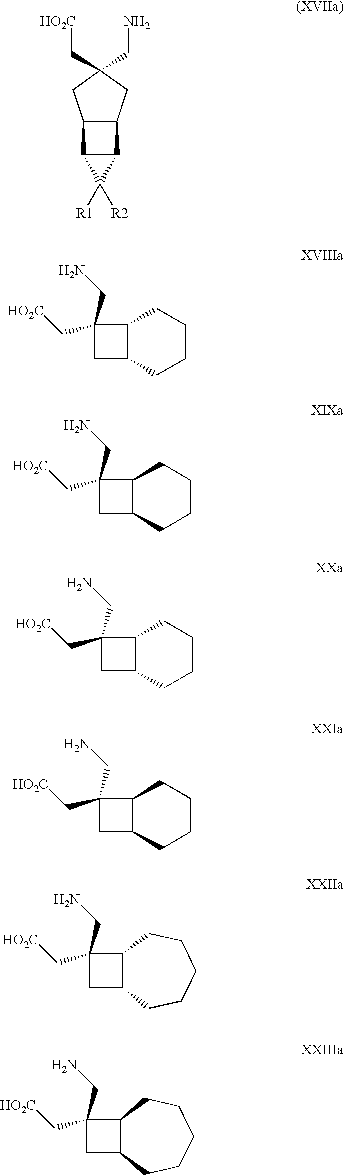

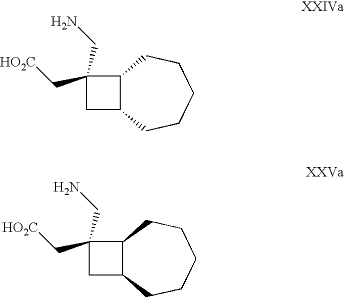

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

81. The method according to Embodiment 80, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

82. The method according to Embodiment 80, wherein the Alpha-2-delta ligand, or a pharmaceutically acceptable salt thereof, is a compound named 3-(1-aminomethyl-cyclohexylmethyl)-4H-[1,2,4]oxadiazo]-5-one hydrochloride.

83. The method according to Embodiment 80, wherein the Alpha-2-delta ligand is a compound named gabapentin.

84. The method according to Embodiment 80, wherein the Alpha-2-delta ligand is a compound which is a pharmaceutically acceptable salt of gabapentin.

85. The method according to Embodiment 80, wherein the Alpha-2-delta ligand is a compound named pregabalin.

86. The method according to Embodiment 80, wherein the Alpha-2-delta ligand is a compound which is a pharmaceutically acceptable salt of pregabalin.

87. The method according to Embodiment 80, wherein the Alpha-2-delta ligand, or a pharmaceutically acceptable salt thereof, is a compound named (3S,4S)-(1-Aminomethyl-3,4-dimethyl-cyclopentyl)-acetic acid, or a pharmaceutically acceptable salt thereof

88. The method according to Embodiment 80, wherein the Alpha-2-delta ligand, or a pharmaceutically acceptable salt thereof, is a compound named (3S,4S)-(1-Aminomethyl-3,4-dimethyl-cyclopentyl)-acetic acid.

89. A method of treating osteoarthritis in a mammal in need thereof, comprising administering to the mammal a therapeutically effective amount of a combination, comprising valdecoxib, or a pharmaceutically acceptable salt thereof, and an Alpha-2-delta ligand, or a pharmaceutically acceptable salt thereof, that is not a compound of Formulas

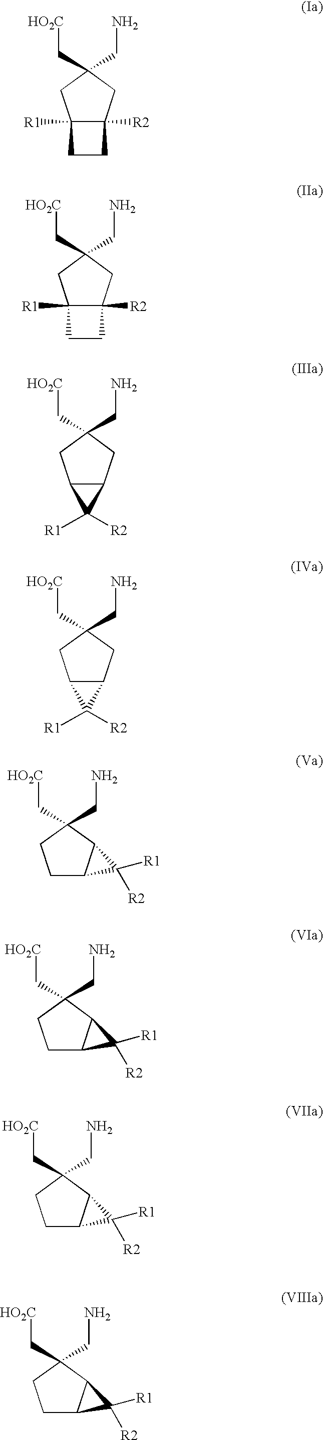

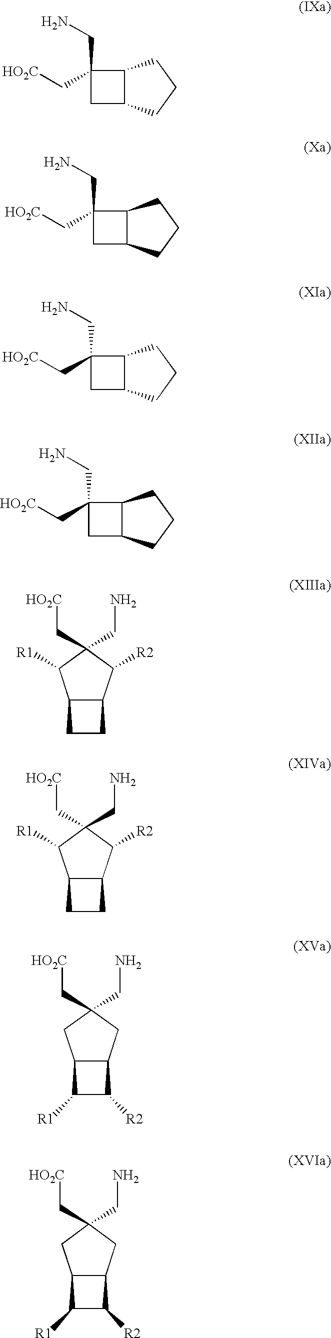

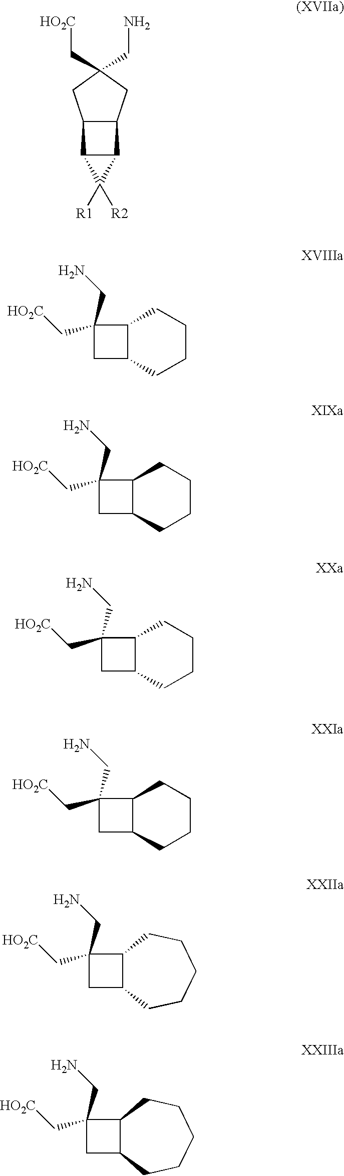

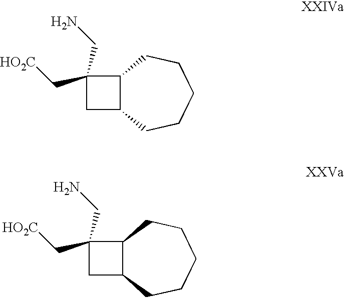

wherein R¹ and R are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

90. The method according to Embodiment 89, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

91. The method according to Embodiment 89, wherein the Alpha-2-delta ligand, or a pharmaceutically acceptable salt thereof, is a compound named 3-(1-aminomethyl-cyclohexylmethyl)-4H-[1,2,4]oxadiazol-5-one hydrochloride.

92. The method according to Embodiment 89, wherein the Alpha-2-delta ligand is a compound named gabapentin.

93. The method according to Embodiment 89, wherein the Alpha-2-delta ligand is a compound which is a pharmaceutically acceptable salt of gabapentin.

94. The method according to Embodiment 89, wherein the Alpha-2-delta ligand is a compound named pregabalin.

95. The method according to Embodiment 89, wherein the Alpha-2-delta ligand is a compound which is a pharmaceutically acceptable salt of pregabalin.

96. The method according to Embodiment 89, wherein the Alpha-2-delta ligand, or a pharmaceutically acceptable salt thereof, is a compound named (3S,4S)-(1-Aminomethyl-3,4-dimethyl-cyclopentyl)-acetic acid, or a pharmaceutically acceptable salt thereof

97. The method according to Embodiment 89, wherein the Alpha-2-delta ligand, or a pharmaceutically acceptable salt thereof, is a compound named (3S,4S)-(1-Aminomethyl-3,4-dimethyl-cyclopentyl)-acetic acid.

98. A method of treating rheumatoid arthritis in a mammal in need thereof, comprising administering to the mammal a therapeutically effective amount of a combination, comprising valdecoxib, or a pharmaceutically acceptable salt thereof, and an Alpha-2-delta ligand, or a pharmaceutically acceptable salt thereof, that is not a compound of Formulas

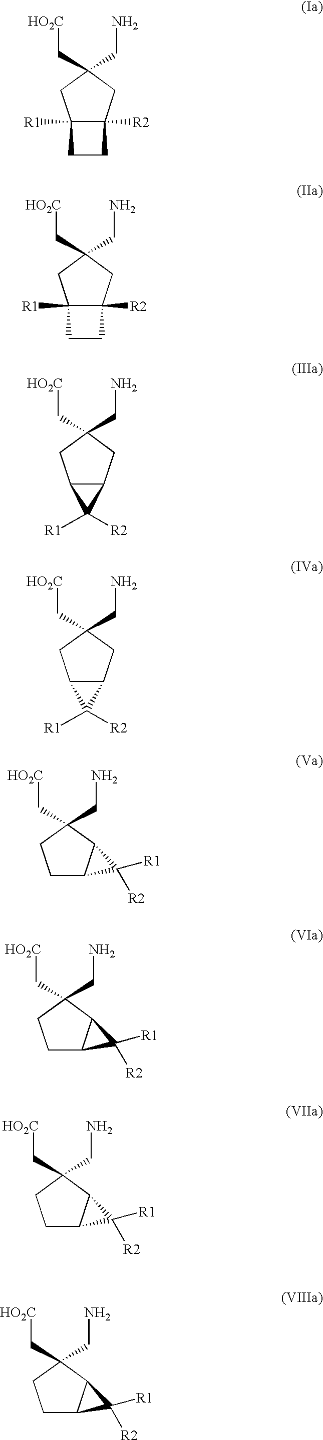

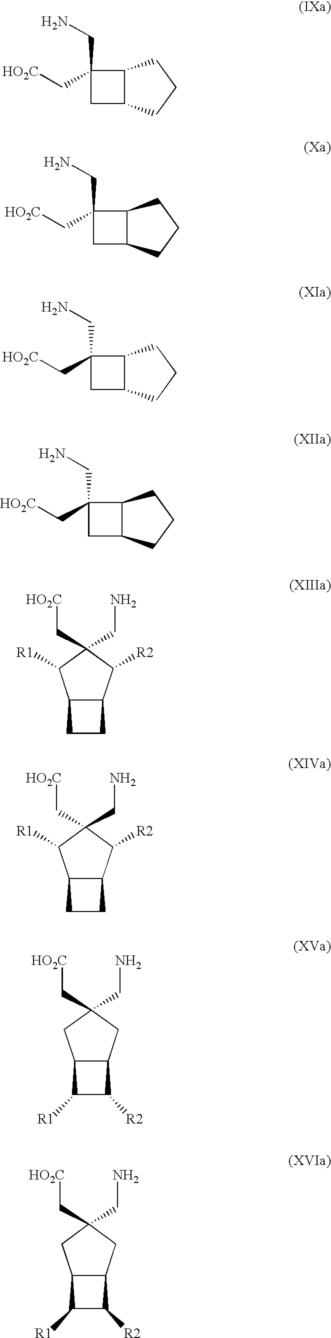

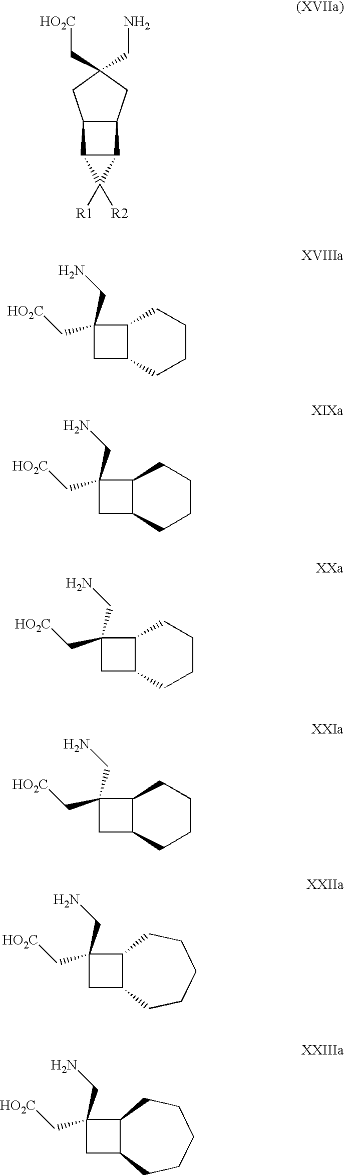

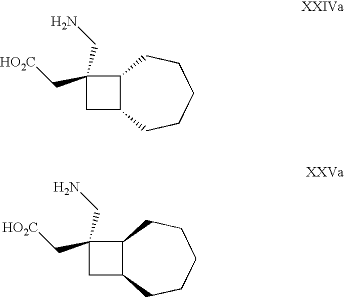

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

99. The method according to Embodiment 98, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

100. The method according to Embodiment 98, wherein the Alpha-2-delta ligand, or a pharmaceutically acceptable salt thereof, is a compound named 3-(1-aminomethyl-cyclohexylmethyl)-4H-[1,2,4]oxadiazol-5-one hydrochloride.

101. The method according to Embodiment 98, wherein the Alpha-2-delta ligand is a compound named gabapentin.

102. The method according to Embodiment 98, wherein the Alpha-2-delta ligand is a compound which is a pharmaceutically acceptable salt of gabapentin.

103. The method according to Embodiment 98, wherein the Alpha-2-delta ligand is a compound named pregabalin.

104. The method according to Embodiment 98, wherein the Alpha-2-delta ligand is a compound which is a pharmaceutically acceptable salt of pregabalin.

105. The method according to Embodiment 98, wherein the Alpha-2-delta ligand, or a pharmaceutically acceptable salt thereof, is a compound named (3S,4S)-(1-Aminomethyl-3,4-dimethyl-cyclopentyl)-acetic acid, or a pharmaceutically acceptable salt thereof

106. The method according to Embodiment 98, wherein the Alpha-2-delta ligand, or a pharmaceutically acceptable salt thereof, is a compound named (3S,4S)-(1-Aminomethyl-3,4-dimethyl-cyclopentyl)-acetic acid.

107. A method of treating psoriatic arthritis in a mammal in need thereof, comprising administering to the mammal a therapeutically effective amount of a combination, comprising valdecoxib, or a pharmaceutically acceptable salt thereof, and an Alpha-2-delta ligand, or a pharmaceutically acceptable salt thereof, that is not a compound of Formulas

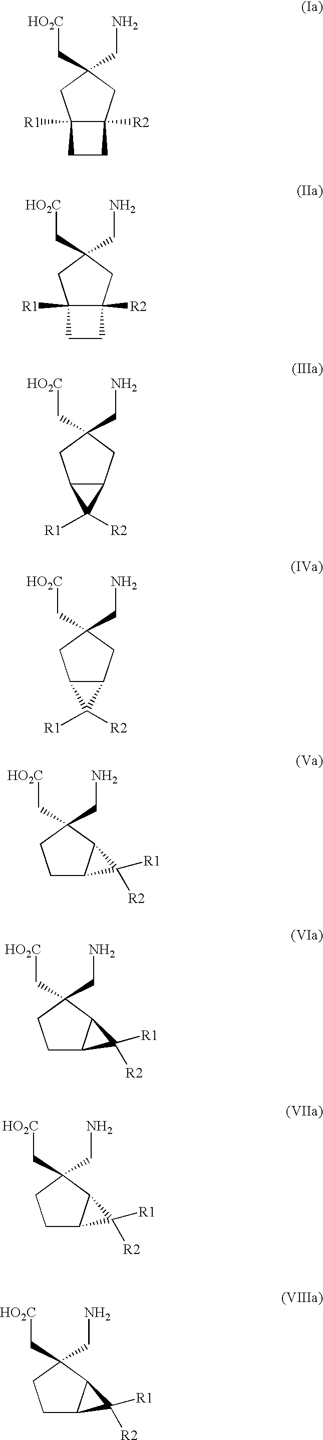

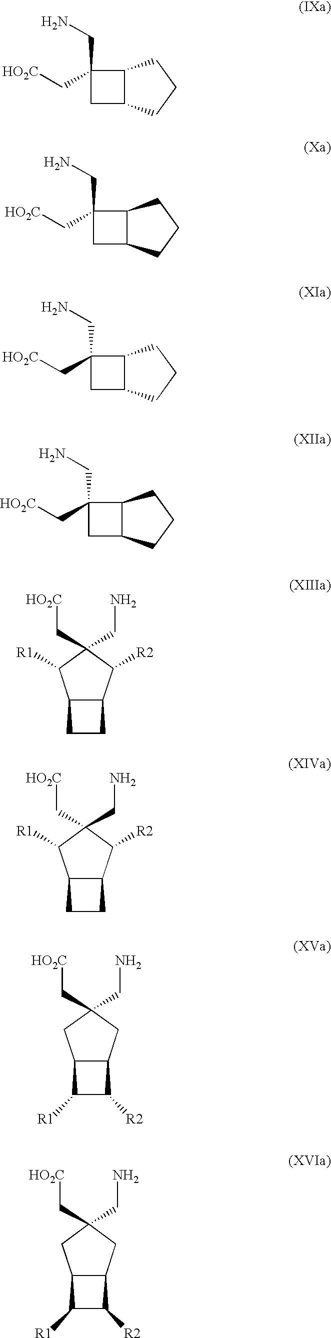

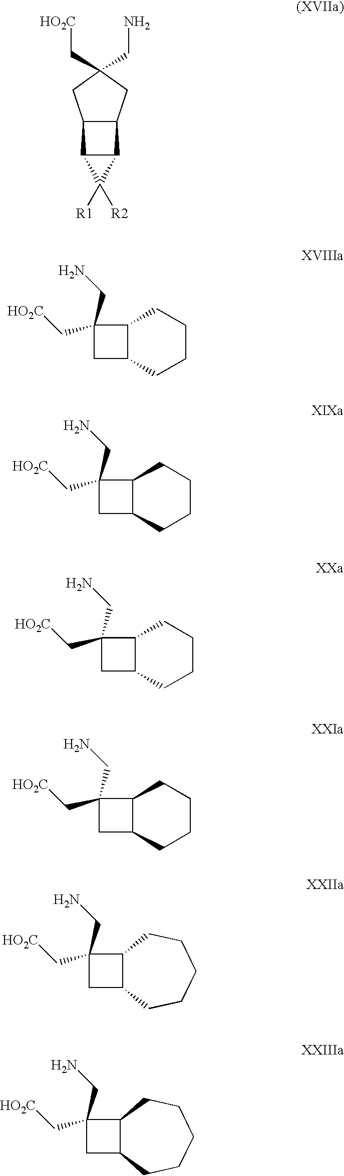

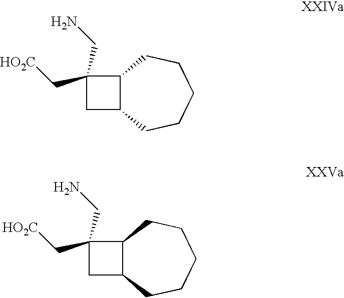

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

108. The method according to Embodiment 107, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

109. The method according to Embodiment 107, wherein the Alpha-2-delta ligand, or a pharmaceutically acceptable salt thereof, is a compound named 3-(1-aminomethyl-cyclohexylmethyl)-4H-[1,2,4]oxadiazol-5-one hydrochloride.

110. The method according to Embodiment 107, wherein the Alpha-2-delta ligand is a compound named gabapentin.

111. The method according to Embodiment 107, wherein the Alpha-2-delta ligand is a compound which is a pharmaceutically acceptable salt of gabapentin.

112. The method according to Embodiment 107, wherein the Alpha-2-delta ligand is a compound named pregabalin.

113. The method according to Embodiment 107, wherein the Alpha-2-delta ligand is a compound which is a pharmaceutically acceptable salt of pregabalin.

114. The method according to Embodiment 107, wherein the Alpha-2-delta ligand, or a pharmaceutically acceptable salt thereof, is a compound named (3S,4S)-(1-Aminomethyl-3,4-dimethyl-cyclopentyl)-acetic acid, or a pharmaceutically acceptable salt thereof

115. The method according to Embodiment 107, wherein the Alpha-2-delta ligand, or a pharmaceutically acceptable salt thereof, is a compound named (3S,4S)-(1-Aminomethyl-3,4-dimethyl-cyclopentyl)-acetic acid.

116. A method of treating pain in a mammal in need thereof, comprising administering to the mammal a therapeutically effective amount of a combination, comprising valdecoxib, or a pharmaceutically acceptable salt thereof, and an Alpha-2-delta ligand, or a pharmaceutically acceptable salt thereof, that is not a compound of Formulas

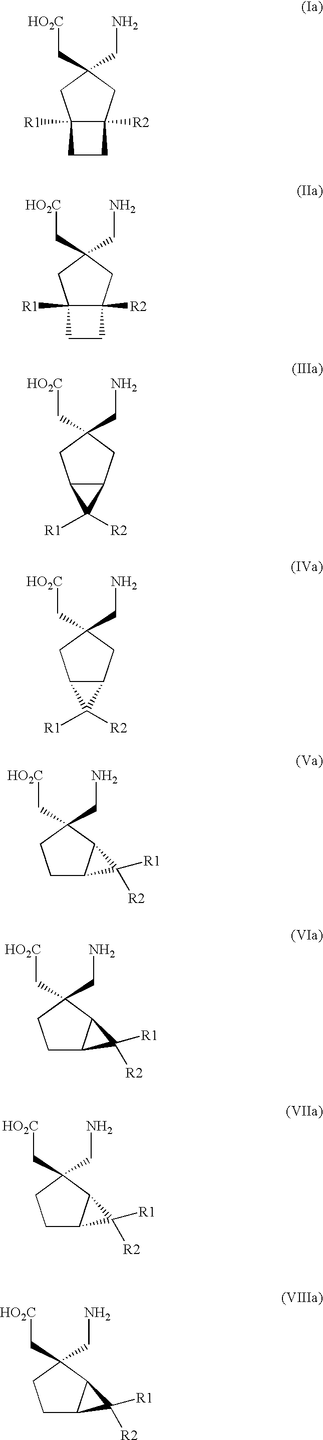

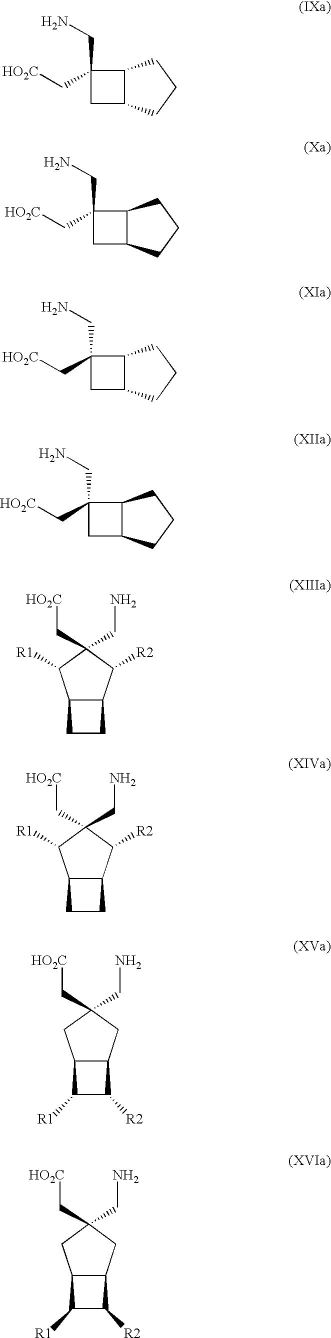

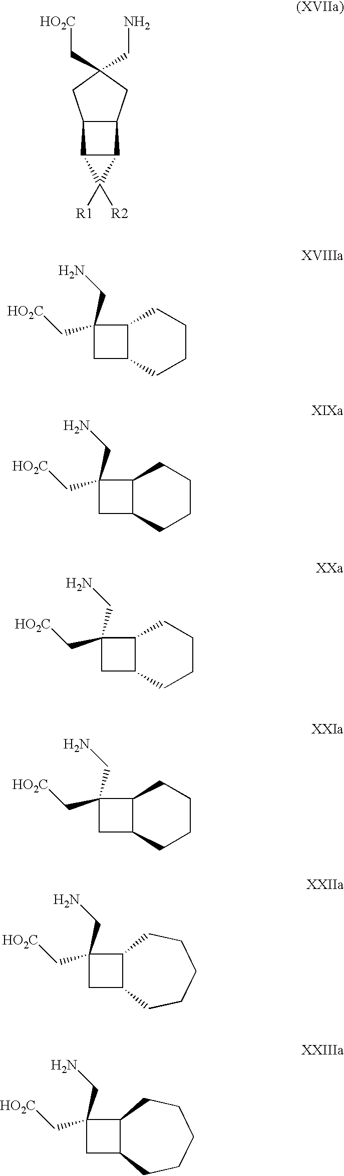

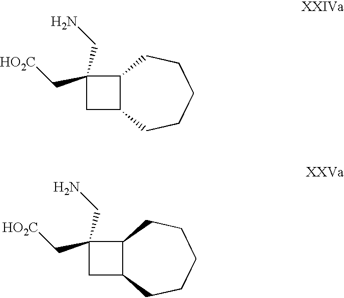

wherein R¹ and R² are each independently selected from H, straight or branched alkyl of 1-6 carbon atoms, cycloalkyl of from 3-6 carbon atoms, phenyl and benzyl, wherein R¹ and R² may not each simultaneously be hydrogen except in the case of the compound of formula (XVIIa).

117. The method according to Embodiment 116, wherein the Alpha-2-delta ligand, or a pharmaceutically acceptable salt thereof, is a compound named 3-(1-aminomethyl-cyclohexylmethyl)-4H-[1,2,4]oxadiazol-5-one, or a pharmaceutically acceptable salt thereof.

118. The method according to Embodiment 116, wherein the Alpha-2-delta ligand, or a pharmaceutically acceptable salt thereof, is a compound named 3-(1-aminomethyl-cyclohexylmethyl)-4H-[1,2,4]oxadiazol-5-one hydrochloride.

119. The method according to Embodiment 116, wherein the Alpha-2-delta ligand is a compound named gabapentin.

120. The method according to Embodiment 116, wherein the Alpha-2-delta ligand is a compound which is a pharmaceutically acceptable salt of gabapentin.

121. The method according to Embodiment 116, wherein the Alpha-2-delta ligand is a compound named pregabalin.

122. The method according to Embodiment 116, wherein the Alpha-2-delta ligand is a compound which is a pharmaceutically acceptable salt of pregabalin.

123. The method according to Embodiment 116, wherein the Alpha-2-delta ligand, or a pharmaceutically acceptable salt thereof, is a compound named (3S,4S)-(1-Aminomethyl-3,4-dimethyl-cyclopentyl)-acetic acid, or a pharmaceutically acceptable salt thereof

124. The method according to Embodiment 116, wherein the Alpha-2-delta ligand, or a pharmaceutically acceptable salt thereof, is a compound named (3S,4S)-(1-Aminomethyl-3,4-dimethyl-cyclopentyl)-acetic acid.

Another invention embodiment is the pharmaceutical composition according to Embodiment 62, wherein the combination is according to any one of Embodiments 1 to 61.

Another invention embodiment is the method according to Embodiment 71, wherein the combination administered is according to any one of Embodiments 1 to 61.

Another invention embodiment is the method according to Embodiment 80, wherein the combination administered is according to any one of Embodiments 1 to 61.

Another invention embodiment is the method according to Embodiment 89, wherein the combination administered is according to any one of Embodiments 1 to 61.

Another invention embodiment is the method according to Embodiment 98, wherein the combination administered is according to any one of Embodiments 1 to 61.

Another invention embodiment is the method according to Embodiment 107, wherein the combination administered is according to any one of Embodiments 1 to 61.

Another invention embodiment is the method according to Embodiment 116, wherein the combination administered is according to any one of Embodiments 1 to 61.

Another invention embodiment is a combination comprising valdecoxib and a compound of Formulas IXA, IXB, or IXC

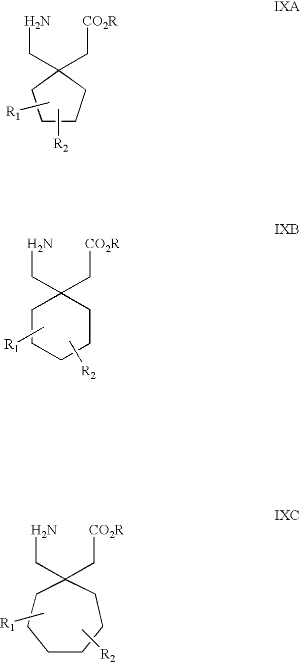

or a pharmaceutically acceptable salt thereof,

wherein:

R is hydrogen or lower alkyl;

R₁ is independently selected from methyl and ethyl; and

R₂ is independently selected from hydrogen, methyl, and ethyl.

Another invention embodiment is a compound of Formulas IXA, IXB, or IXC, or a pharmaceutically acceptable salt thereof, selected from:

(1-aminomethyl-3-methylcyclohexyl) acetic acid,

(1-aminomethyl-3-methylcyclopentyl) acetic acid, and

(1-aminomethyl-3,4-dimethylcyclopentyl) acetic acid, or a pharmaceutically acceptable salt thereof.

Another invention embodiment is a combination comprising valdecoxib and a compound of Formula II as defined above, or a pharmaceutically acceptable salt thereof, where R₂ and R₃ are both hydrogen, and R₁ is —(CH₂)₀₋₂-iC₄H₉ as an (R), (S), or (R,S) isomer.

Another invention embodiment is a combination comprising valdecoxib and a compound of Formula II as defined above, or a pharmaceutically acceptable salt thereof, selected from: (R/S)-3-aminomethyl-5-methyl-hexanoic acid, (R)-3-(aminomethyl)-5-methylhexanoic acid, and (S)-3-(aminomethyl)-5-methylhexanoic acid, or a pharmaceutically acceptable salt thereof. The compound (S)-3-(aminomethyl)-5-methylhexanoic acid is also known generically as pregabalin, “CI-1008”, and “S-(+)-3-IBG.”

Other invention embodiments include:

A combination comprising valdecoxib and a compound named:

[(1R,5R,6S)-6-(Aminomethyl)bicyclo[3.2.0]hept-6-yl]acetic acid;

or a pharmaceutically acceptable salt thereof.

A combination comprising valdecoxib and a compound named:

[(1R,5R,6S)-6-(Aminomethyl)bicyclo[3.2.0]hept-6-yl]acetic acid.

Any one of the above embodiments of a pharmaceutical composition, wherein the COX-2 inhibitor is in unit dosage form in an amount of from 5 milligrams to 750 milligrams and the Alpha-2-delta ligand is in unit dosage form in an amount of from 10 milligrams to 1000 milligrams.

Any one of the above embodiments of a pharmaceutical composition, wherein the COX-2 inhibitor is in unit dosage form in an amount of from 10 milligrams to 500 milligrams and the Alpha-2-delta ligand is in unit dosage form in an amount of from 10 milligrams to 750 milligrams.

Any one of the above embodiments of a pharmaceutical composition, wherein the COX-2 inhibitor is in unit dosage form in an amount of from 20 milligrams to 250 milligrams and the Alpha-2-delta ligand is in unit dosage form in an amount of from 10 milligrams to 500 milligrams.

Any one of the above embodiments of a pharmaceutical composition, wherein the COX-2 inhibitor is in unit dosage form in an amount of from 25 milligrams to 200 milligrams and the Alpha-2-delta ligand is in unit dosage form in an amount of from 10 milligrams to 250 milligrams.

Any one of the above embodiments of a pharmaceutical composition, wherein the COX-2 inhibitor is in unit dosage form in an amount of from 25 milligrams to 150 milligrams and the Alpha-2-delta ligand is in unit dosage form in an amount of from 10 milligrams to 200 milligrams.

Any one of the above embodiments of a method of treating, wherein the COX-2 inhibitor is in unit dosage form in an amount of from 5 milligrams to 750 milligrams and the Alpha-2-delta ligand is in unit dosage form in an amount of from 10 milligrams to 1000 milligrams.

Any one of the above embodiments of a method of treating, wherein the COX-2 inhibitor is in unit dosage form in an amount of from 10 milligrams to 500 milligrams and the Alpha-2-delta ligand is in unit dosage form in an amount of from 10 milligrams to 750 milligrams.

Any one of the above embodiments of a method of treating, wherein the COX-2 inhibitor is in unit dosage form in an amount of from 20 milligrams to 250 milligrams and the Alpha-2-delta ligand is in unit dosage form in an amount of from 10 milligrams to 500 milligrams.

Any one of the above embodiments of a method of treating, wherein the COX-2 inhibitor is in unit dosage form in an amount of from 25 milligrams to 200 milligrams and the Alpha-2-delta ligand is in unit dosage form in an amount of from 10 milligrams to 250 milligrams.

Any one of the above embodiments of a method of treating, wherein the COX-2 inhibitor is in unit dosage form in an amount of from 25 milligrams to 150 milligrams and the Alpha-2-delta ligand is in unit dosage form in an amount of from 10 milligrams to 200 milligrams.

A pharmaceutical composition, comprising valdecoxib in unit dosage form in an amount of from 1 milligram to 50 milligrams, and pregabalin in unit dosage form in an amount of from 10 milligrams to 600 milligrams.

A pharmaceutical composition, comprising valdecoxib in unit dosage form in an amount of from 5 milligrams to 50 milligrams, and pregabalin in unit dosage form in an amount of from 10 milligrams to 300 milligrams.

A pharmaceutical composition, comprising valdecoxib in unit dosage form in an amount of from 5 milligrams to 25 milligrams, and pregabalin in unit dosage form in an amount of from 25 milligrams to 300 milligrams.

A pharmaceutical composition, comprising valdecoxib in unit dosage form in an amount of from 5 milligrams to 25 milligrams, and pregabalin in unit dosage form in an amount of from 25 milligrams to 200 milligrams.

A pharmaceutical composition, comprising valdecoxib in unit dosage form in an amount of from 1 milligram to 5 milligrams, and pregabalin in unit dosage form in an amount of from 25 milligrams to 100 milligrams.

A pharmaceutical composition, comprising valdecoxib and an Alpha-2-delta ligand named [(1R,5R,6S)-6-(Aminomethyl)bicyclo[3.2.0]hept-6-yl]acetic acid, or a pharmaceutically acceptable salt thereof.

A pharmaceutical composition, comprising valdecoxib and an Alpha-2-delta ligand named [(1R,5R,6S)-6-(Aminomethyl)bicyclo[3.2.0]hept-6-yl]acetic acid.

Any one of the above embodiments of a method of treating, wherein the COX-2 inhibitor is valdecoxib in unit dosage form in an amount of from 1 milligrams to 50 milligrams, and the Alpha-2-delta ligand is pregabalin in unit dosage form in an amount of from 10 milligrams to 600 milligrams.

Any one of the above embodiments of a method of treating, wherein the COX-2 inhibitor is valdecoxib in unit dosage form in an amount of from 5 milligrams to 50 milligrams, and the Alpha-2-delta ligand is pregabalin in unit dosage form in an amount of from 10 milligrams to 300 milligrams.

Any one of the above embodiments of a method of treating, wherein the COX-2 inhibitor is valdecoxib in unit dosage form in an amount of from 5 milligrams to 25 milligrams, and the Alpha-2-delta ligand is pregabalin in unit dosage form in an amount of from 25 milligrams to 300 milligrams.

Any one of the above embodiments of a method of treating, wherein the COX-2 inhibitor is valdecoxib in unit dosage form in an amount of from 5 milligrams to 25 milligrams, and the Alpha-2-delta ligand is pregabalin in unit dosage form in an amount of from 25 milligrams to 250 milligrams.

Any one of the above embodiments of a method of treating, wherein the COX-2 inhibitor is valdecoxib in unit dosage form in an amount of from 1 milligrams to 5 milligrams, and the Alpha-2-delta ligand is pregabalin in unit dosage form in an amount of from 25 milligrams to 100 milligrams.

Another invention embodiment is a combination according to any one of the above combination embodiments, wherein the COX-2 inhibitor is celecoxib or the COX-2 inhibitor which is valdecoxib is replaced by celecoxib.

Another invention embodiment is a combination according to any one of the above combination embodiments, wherein the COX-2 inhibitor is parecoxib or the COX-2 inhibitor which is valdecoxib is replaced by parecoxib.

Another invention embodiment is a combination according to any one of the above combination embodiments, wherein the COX-2 inhibitor is any one of the selective COX-2 inhibitors identified below except valdecoxib, parecoxib, and celecoxib, or the COX-2 inhibitor which is valdecoxib is replaced by any one of the selective COX-2 inhibitors identified below except valdecoxib, parecoxib, and celecoxib.

Any one of the above embodiments of a combination, wherein the COX-2 inhibitor, or a pharmaceutically acceptable salt thereof, is in an amount of from 5 milligrams to 1000 milligrams, and the Alpha-2-delta ligand, or a pharmaceutically acceptable salt thereof, is in an amount of from 5 milligrams to 1000 milligrams.

In a further embodiment, the patient being treated for cartilage damage does not have pain in a joint. In another embodiment, the patient being treated for cartilage damage also has pain in a joint.

In another further embodiment, the patient being treated for inflammation has joint inflammation.

In another further embodiment, the patient being treated for pain has joint pain.

### DETAILED DESCRIPTION OF THE INVENTION

As noted above, the invention combination comprises a selective inhibitor of COX-2, or a pharmaceutically acceptable salt thereof, and any Alpha-2-delta ligand, or a pharmaceutically acceptable salt thereof. For the purposes of the instant invention, an Alpha-2-delta ligand is any compound structurally analogous to gamma-aminobutyric acid (“GABA”), as illustrated and described herein, that provides a therapeutic effect on the disease being treated. In other words, an Alpha-2-delta ligand is a compound that, when administered to a patient according to the method of the instant invention, provides a compound in vivo with a bioactive form that has a similar electronic structure to, but different atoms than, the bioactive form of GABA. For example, administration of GABA itself (gamma amino butyric acid), or a salt thereof, would provide a bioactive agent in vivo that would be different from the bioactive form provided by administration of an Alpha-2-delta ligand such as gabapentin. This is illustrated in Scheme 1 below, which assumes physiological pH 7.4.

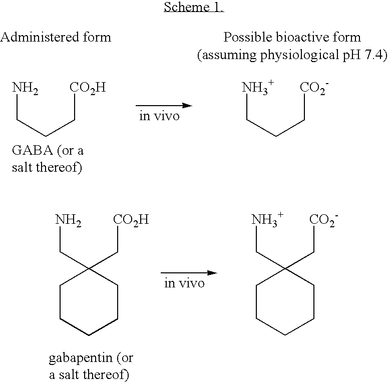

In Scheme 1, there is one predominant bioactive form of GABA, or a salt thereof, and one predominant bioactive form of gabapentin, or a salt thereof. Further, the bioactive form of GABA, and salts thereof, shares some, but not all, atoms and bonds with the bioactive form of gabapentin, and salts thereof. An Alpha-2-delta ligand as the term is used herein is thus not gamma-aminobutyric acid, or a salt of gamma-aminobutyric acid.

Illustrative examples of Alpha-2-delta ligands provided in the invention embodiments are described above in the invention embodiments, and in the patents and patent applications referenced below. For further illustration purposes only, an Alpha-2-delta ligand also includes, but is not limited to, a compound of Formula (A)

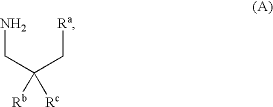

or a pharmaceutically acceptable salt thereof,

wherein:

R^{a} is COOH, C(O)N(H)OH, SO₃H, PO₃H₂, —NHCOR¹², wherein R¹² is selected from straight or branched unsubstituted alkyl of from 1 to 6 carbons, benzyl, and phenyl, —NHSO₂R¹⁵, —SO₂NHR¹⁵, wherein R¹⁵ is a straight or branched unsubstituted alkyl group of from 1 to 6 carbons or a trifluoromethyl, a 5-membered or 6-membered monocyclic heterocyclic group containing carbon atoms and from 1 to 4 heteroatoms selected from oxygen (0 or 1), sulfur (0 or 1), and nitrogen (0 to 4), wherein one of the heteroatoms is bonded to a hydrogen atom, or a 8-membered or 9-membered bicyclic heterocyclic group containing carbon atoms and from 1 to 4 heteroatoms selected from oxygen (0 or 1 total), sulfur (0 or 1 total), and nitrogen (0 to 4 total), wherein one of the heteroatoms is bonded to a hydrogen atom;

R^{b} and R^{c} are independently hydrogen, C₁-C₁₅ alkyl, C₃-C₁₅ cycloalkyl, or a heterocycloalkyl containing from 2 to 14 carbon atoms and 1 heteroatom selected from O, S, and NCH₃; or

R^{b} and R^{c} are taken together with the carbon atom to which they are both attached to form a C₃-C₁₅ cycloalkylene, a heterocycloalkylene containing from 2 to 14 carbon atoms and 1 heteroatom selected from O, S, and NCH₃, a C₅-C₁₅ bicycloalkylene, or a heterobicycloalkylene containing from 4 to 14 carbon atoms and 1 heteroatom selected from O, S, and NCH₃; and

with the proviso that R^{b} and R^{c} are not both hydrogen.

Preferred heterocyclic groups for R^{a} are

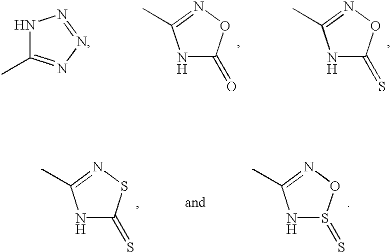

A compound that is an Alpha-2-delta ligand may be readily identified by one of ordinary skill in the pharmaceutical or medical arts by assaying the Alpha-2-delta ligand in any number of well-known assays for measuring binding affinity at Alpha-2-delta receptors. One such Alpha-2-delta receptor binding assay is described by Chauhan N. Suman, L. Webdale, D. R. Hill, and G. N. Woodruff, “Characterization of [3H]gabapentin binding to a novel site in rat brain: homogenate binding studies”, Eur. J. Pharmacol., 1993;244(3):293-301.

Further, an Alpha-2-delta ligand having an anti-inflammatory, an analgesic, or a cartilage damage inhibiting effect, or any combination of these effects, may be readily identified by one of ordinary skill in the pharmaceutical or medical arts by assaying the Alpha-2-delta ligand in any number of well known assays for measuring determining the Alpha-2-delta ligand's effects on cartilage damage, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of an Alpha-2-delta ligand or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content.

Further, in vivo assays to assay cartilage damage may be performed as follows: an amount of an Alpha-2-delta ligand or control vehicle may be administered with a cartilage damaging agent to an animal, and the effects of the Alpha-2-delta ligand being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content. Conventional imaging techniques such as X-ray or magnetic resonance imaging may also be used to assess cartilage damage in a joint.

Several methods of identifying an Alpha-2-delta ligand with cartilage damage inhibiting properties are described below. The amount to be administered in an assay to identify an Alpha-2-delta ligand is dependent upon the particular assay employed, but in any event is not higher than the well known maximum amount of a compound that the particular assay can effectively accommodate.

Similarly, Alpha-2-delta ligands having pain-alleviating properties may be identified using any one of a number of in vivo animal models of pain. For example, a method for identifying certain Alpha-2-delta ligands having pain-alleviating effects in a static or dynamic model of allodynia is known (See M. J. Field, et al., “Gabapentin and pregabalin, but not morphine and amitriptyline, block both static and dynamic components of mechanical allodynia induced by streptozocin in the rat”, Pain, 1999;80:391-398.)

Still similarly, Alpha-2-delta ligands having anti-inflammatory properties may be identified using any one of a number of in vivo animal models of inflammation. For example, for an example of inflammation models, see U.S. Pat. No. 6,329,429, which is incorporated herein by reference.

Still similarly, Alpha-2-delta ligands having anti-arthritic properties may be identified using any one of a number of in vivo animal models of arthritis. For example, for an example of arthritis models, see also U.S. Pat. No. 6,329,429.

Any Alpha-2-delta ligand is readily available, either commercially, or by synthetic methodology, well known to those skilled in the art of organic chemistry. For example, an Alpha-2-delta ligand of Formula I, including gabapentin, and pharmaceutically acceptable salts thereof, as described above, and preparations thereof, are described in U.S. Pat. No. 4,024,175 and its divisional U.S. Pat. No. 4,087,544, which are both incorporated herein by reference.

Further, Alpha-2-delta ligands of Formula II, including pregabalin, and their pharmaceutically acceptable salts, as described above, and preparations thereof, are described in U.S. Pat. No. 5,563,175, which is incorporated herein by reference.

The terms are as defined below or as they otherwise occur in the specification.

It should be appreciated that the term “pregabalin” means an Alpha-2-delta ligand in Phase III clinical trials for the treatment of convulsions and neuropathic pain. Pregabalin is administered either BID or TID in these trials at total daily dosages of from 150 milligrams-per-day to 600 milligrams-per-day. Pregabalin, also known as (S)-3-(aminomethyl)-5-methylhexanoic acid, has the structure drawn below:

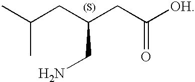

It should be appreciated that diastereomers and enantiomers of compounds of Formula II as defined above, or a pharmaceutically acceptable salt thereof, can be utilized in the invention combination.

Alpha-2-delta ligands of Formula III, IIIC, IIIF, IIIG, or IIIH, and their pharmaceutically acceptable salts, as described above, and preparations thereof, are described in PCT International Application Publication No. WO 99/31075, which is herein incorporated by reference.

Alpha-2-delta ligands of Formula IV, and their pharmaceutically acceptable salts, as described above, and preparations thereof, are described in PCT International Application Publication No. WO 00/76958, which is herein incorporated by reference.

Alpha-2-delta ligands of Formulas (1A) and (1B), and their pharmaceutically acceptable salts, as described above, and preparations thereof, are described in PCT International Application Publication No. WO 99/31074, which is herein incorporated by reference.

Alpha-2-delta ligands of Formulas V, VI, VII, and VIII, and their pharmaceutically acceptable salts, as described above, and preparations thereof, are described in PCT International Application Publication No. WO 01/28978, which is herein incorporated by reference.

Alpha-2-delta ligands of Formula (1D) and (1E), and their pharmaceutically acceptable salts, as described above, and preparations thereof, are described in PCT International Application No. WO 99/31057, which is herein incorporated by reference.

Alpha-2-delta ligands of Formula

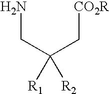

and their pharmaceutically acceptable salts, as described above, and preparations thereof, are described in PCT International Application No. WO 98/17627, which is herein incorporated by reference.

Alpha-2-delta ligands of Formulas (1), (2), (3), (4), (5), (6), (7), and (8), and their pharmaceutically acceptable salts, as described above, and preparations thereof, are described in PCT International Application No. WO 99/61424, which is herein incorporated by reference.

It should also be appreciated that in Formula (1) described above, R cannot be sulfonic acid when m is 2 and n is 1. (Suman-Chaulan N., et al., European Journal of Pharmacology, 1993;244:293-301.) Alpha-2-delta ligands of Formulas (9) and (9A), and their pharmaceutically acceptable salts, and preparations thereof, are described in PCT International Application No. WO 99/21824, which is herein incorporated by reference.

Other Alpha-2-delta ligands useful in the invention combination, pharmaceutical compositions comprising the invention combination, and methods of using the invention combination, and preparations thereof, include Alpha-2-delta ligands taught in WO 02/22568 A1 and WO 02/30871 A1, which are hereby incorporated by reference herein.

As mentioned above, Alpha-2-delta ligands useful in the invention combination include those described in U.S. Pat. No. 4,024,175 and its divisional U.S. Pat. No. 4,087,544, U.S. Pat. No. 5,563,175, and PCT International Application Publication Nos. WO 99/31075, WO 00/76958, WO 99/31074, WO 01/28978, WO 99/31057, WO 98/17627, WO 99/61424, WO 99/21824, WO 02/22568, and WO 02/30871.

All U.S. patents and WO publications referenced above are hereby incorporated by reference.

As described above, Alpha-2-delta ligands that are excluded from the invention combination are those described in WO 02/085839.

It should be appreciated that the compound named (3S,4S)-(1-Aminomethyl-3,4-dimethyl-cyclopentyl)-acetic acid is also known by the names (S,S)-(1-Aminomethyl-3,4-dimethyl-cyclopentyl)-acetic acid and (3S,4S)-1-(aminomethyl)-cyclopentaneacetic acid. The compound named (3S,4S)-(1-Aminomethyl-3,4-dimethyl-cyclopentyl)-acetic acid has the structure drawn immediately below:

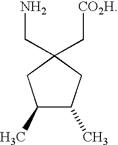

It should be appreciated that the compound named [(1R,5R,6S)-6-(Aminomethyl)bicyclo[3.2.0]hept-6-yl]acetic acid has the structure drawn immediately below:

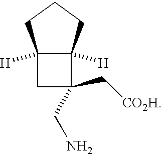

For the purposes of this invention, a selective inhibitor of COX-2 includes a compound, or a pharmaceutically acceptable salt thereof, selected from:

ABT-963;

Valdecoxib;

BMS-347070;

Celecoxib;

Tilacoxib;

The compound of formula (B)

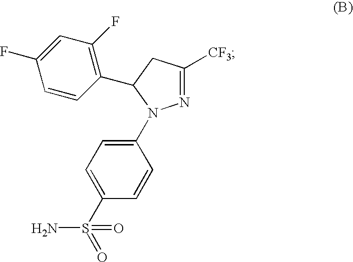

CS-502 [Chemical Abstracts Service Registry Number (“CAS Reg. No.”) 176429-82-6];

(6aR,10aR)-3-(1,1-dimethylheptyl)-6a,7,10,10a-tetrahydro-1-hydroxy-6,6-dimethyl-6H-dibenzo[b,d]pyran-9-carboxylic acid (“CT-3”);

CV-247;

2(5H)-Furanone, 5,5-dimethyl-3-(1-methylethoxy)-4-[4-(methylsulfonyl)phenyl]-(“DFP”);

Etoricoxib,

GW-406381;

Tiracoxib;

Meloxicam;

Nimesulide;

2-(Acetyloxy)benzoic acid, 3-[(nitrooxy)methyl]phenyl ester (“NCX-4016”);

Parecoxib;

P54 (CAS Reg. No. 130996-28-0);

Rofecoxib;

RevlMiD;

2,6-Bis(1,1-dimethylethyl)-4-[(E)-(2-ethyl-1,1-dioxo-5-isothiazolidinylidene)methyl]phenol (“S-2474”);

5(R)-Thio-6-sulfonamide-3(2H)-benzofuranone (“SVT-2016”); and

N-[3-(Formylamino)-4-oxo-6-phenoxy-4H-1-benzopyran-7-yl]-methanesulfonamide (“T-614”), or a pharmaceutically acceptable salt thereof.

The term “valdecoxib” means the compound named 4-(5-methyl-3-phenyl-4-isoxazolyl)-benzenesulfonamide, which is described in U.S. Pat. Nos. 5,633,272; 5,859,257; and 5,985,902, which are hereby incorporated by reference herein. Valdecoxib has been approved by the FDA for treating osteoarthritis, rheumatoid arthritis, dysmenorrhea, and general pain, and is marketed under the tradename “Bextra”. Valdecoxib is in clinical trials for the treatment of migraine. Valdecoxib, which is preferred over a pharmaceutically acceptable salt thereof, has the structure drawn below:

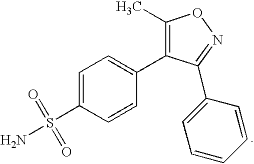

The term “rofecoxib” means the compound named 4-[4-(methylsulfonyl)phenyl]-3-phenyl-2(5H)-furanone. Rofecoxib has been approved by the FDA for treatment of osteoarthritis, general pain, and post-operative pain, and is preregistered for treatment of rheumatoid arthritis. Rofecoxib is marketed under the tradename “Vioxx”. Rofecoxib is currently in clinical trials for treatment of juvenile rheumatoid arthritis, colorectal cancer, colorectal cancer prevention, polyposis-familial adenomatus (“FAP”), and polyposis-spontaneous adenomatous-prevention. Rofecoxib has the structure drawn below:

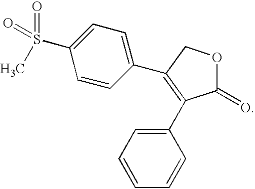

The term “celecoxib” means the compound named 4-(5-(4-methylphenyl)-3-(trifluoromethyl)-1H-pyrazol-1-yl)-benzenesulfonamide. Celecoxib is currently approved by the FDA for the treatment of osteoarthritis, rheumatoid arthritis, and Polyposis-familial adenomatus. Celecoxib is marketed under the tradename “Celebrex”. Celecoxib is currently in clinical trials for the treatment of bladder cancer, chemopreventative-lung cancer, and post-operative pain, and is registered for the treatment of dysmenorrhea. Celecoxib has the structure drawn below:

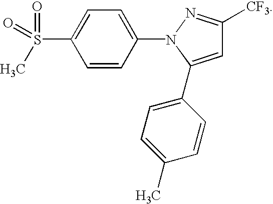

The term “selective” as applied herein to COX-2 inhibitors means a ratio of IC₅₀ for a compound with COX-1 divided by a ratio of IC₅₀ for the compound with COX-2 that is greater than, or equal to, 5, where the ratios are determined in one or more of the in vitro, in vivo, or ex vivo assays described below. All that is required to identify a selective COX-2 inhibitor useful in the combination of the present invention is to assay a compound in one of the pairs of assays described in Biological Methods 3 to 6 below. Preferred selective COX-2 inhibitors have a selectivity greater than 5 fold versus COX-1 in the assay described in Biological Method 3 below.

For the purposes of this invention, the term “arthritis” includes osteoarthritis, rheumatoid arthritis, degenerative joint disease, spondyloarthropathies, gouty arthritis, systemic lupus erythematosus, juvenile arthritis, and psoriatic arthritis. An Alpha-2-delta ligand having an anti-arthritic effect is a compound as defined above that inhibits the progress, prevents further progress, or reverses progression, in part or in whole, of any one or more symptoms of any one of the arthritic diseases and disorders listed above.

Other mammalian diseases and disorders which are treatable by administration of an invention combination alone, or contained in a pharmaceutical composition as defined below, include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

The term “C₁-C₁₅ alkyl” means an unsubstituted straight or branched alkyl group having from 1 to 15 carbon atoms, including, but not limited to, methyl, butyl, iso-pentyl, 4-nonyl, 4,4,5,6-tetramethyldecyl, and the like.

The phrase “lower alkyl” means a straight or branched alkyl group or radical having from 1 to 6 carbon atoms, and includes methyl, ethyl, n-propyl, i-propyl, n-butyl, i-butyl, sec-butyl, tert-butyl, n-pentyl, n-hexyl, and the like.

The phrase “straight or branched alkyl of 1-6 carbon atoms” means the same thing as the phrase “lower alkyl”, as defined immediately above.

The term “alkyl” is a straight or branched group of from 1 to 8 carbon atoms, unless stated otherwise, including but not limited to methyl, ethyl, propyl, n-propyl, isopropyl, butyl, 2-butyl, tert-butyl, and octyl. Alkyl can be unsubstituted or substituted by hydroxy or from 1 to 3 fluorine atoms. Preferred groups are methyl and ethyl.

The term “alkenyl” is a straight or branched group of from 2 to 8 carbon atoms containing 1 or 2 or 3 double bonds including but not limited to ethenyl, propen-1-yl, propen-2-yl, propen-3-yl, 1-hexen-3-yl, and hept-1,3-dien-7-yl. Alkenyl can be unsubstituted or substituted by from 1 to 3 fluorine atoms.

The term “C₃-C₁₅ cycloalkyl” means a monocyclic carbocyclic group containing from 3 to 15 carbon atoms, which is unsubstituted or substituted with 1 or 2 lower alkyl groups. C₃-C₁₅ cycloalkyl includes, but is not limited to, cyclopropyl, cyclononyl, and cyclopentadecyl.

The term “cycloalkyl” means a cyclic group of from 3 to 7 carbon atoms including but not limited to cyclopropyl, cyclobutyl, and cycloheptyl.

The phrase “cycloalkyl of from 3-6 carbon atoms” means a cyclic group of from 3 to 6 carbon atoms including cyclopropyl, cyclobutyl, cyclopentyl, and cyclohexyl.

The term “heterocycloalkyl” means a monocyclic group containing from 2 to 14 carbon atoms and 1 heteroatom selected from O, S, and NCH₃, which is unsubstituted or substituted with 1 or 2 lower alkyl groups. Heterocycloalkyl includes, but is not limited to, 1-methyl-aziridin-2-yl, 1-methyl-piperidin-4-yl, and 5-oxacyclopentadecyl.

The term “C₃-C₁₅ cycloalkylene” means a monocyclic carbocyclic gem diradical containing from 3 to 15 carbon atoms, which is unsubstituted or substituted with 1 or 2 lower alkyl groups. C₃-C₁₅ cycloalkylene includes, but is not limited to, 1,1-cyclopropylene, 1,1-cyclononylene, and 1,1-cyclopentadecylene.

The term “heterocycloalkylene” means a monocyclic gem diradical containing from 2 to 14 carbon atoms and 1 heteroatom selected from O, S, and NCH₃, including, but not limited to, 1-methyl-2,2-aziridinylene, 1-methyl-4,4-piperidinylene, and 5-oxa-1,1-cyclopentadecylene.

The term “C₅-C₁₅ bicycloalkylene” means a bicyclic carbocyclic gem diradical containing from 5 to 15 carbon atoms, which is unsubstituted or substituted with 1 or 2 lower alkyl groups. C₅-C₁₅ bicycloalkylene includes, but is not limited to, 2-bicyclo[2.2.1]pentylene, 3-bicyclo[3.3.1]nonylene, and 14-bicyclo[11.2.0]pentadecylene.

The term “heterobicycloalkylene” means a bicyclic gem diradical containing from 4 to 14 carbon atoms and 1 heteroatom selected from O, S, and NCH₃, which is unsubstituted or substituted with 1 or 2 lower alkyl groups. heterobicycloalkylene includes, but is not limited to, 1-aza-2-bicyclo[2.2.1]pentylene, 2-thia-3-bicyclo[3.3.1]nonylene, and 14-methyl-14-aza-15-bicyclo[11.2.0]pentadecylene.

The benzyl and phenyl groups may be unsubstituted or substituted with from 1 to 3 groups each independently selected from halogen, especially fluoro, alkoxy, alkyl, and NH₂.

Halogen includes fluorine, chlorine, bromine, and iodine.

The term “alkoxy” means the group —O-alkyl wherein alkyl is as defined above.

The terms used to define the invention of compounds of Formulas (1A), (1B), III, IIIC, IIIF, IIIG, and IIIH are as described below.

Sulfonamides are those of formula —NHSO₂R¹⁵ or —SO₂NHR¹⁵ wherein R¹⁵ is a straight or branched alkyl group of from 1 to 6 carbons or a trifluoromethyl.

Amides are compounds of formula —NHCOR¹² wherein R¹² is straight or branched alkyl of from 1 to 6 carbons, benzyl, and phenyl.

Phosphonic acids are —PO₃H₂.

Sulfonic acids are —SO₃H.

Hydroxamic acid is

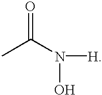

Heterocycles are groups of from 1 to 2 rings, with from 1 to 6 heteroatoms selected from oxygen, nitrogen, and sulfur.

Preferred heterocycles are

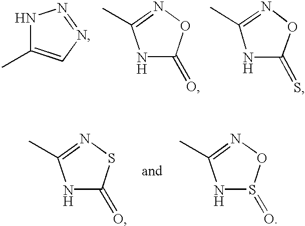

The term alkyl is a straight or branched group of from 1 to 11 carbon atoms including but not limited to methyl, ethyl, propyl, n-propyl, isopropyl, butyl, 2-butyl, tert-butyl, pentyl, hexyl, and n-hexyl, heptyl, octyl, nonyl, decyl, and undecyl except as where otherwise stated.

The cycloalkyl groups are from 3 to 8 carbons and are cyclopropyl, cyclobutyl, cyclopentyl, cyclohexyl, cycloheptyl, and cyclooctyl unless otherwise stated.

The benzyl and phenyl groups may be unsubstituted or substituted by from 1 to 3 substituents selected from hydroxy, carboxy, carboalkoxy, halogen, CF₃, nitro, alkyl, and alkoxy. Preferred are halogens.

Alkoxy is as defined above for alkyl.

Halogen is fluorine, chlorine, and bromine and preferred are fluorine and chlorine.

Carboalkoxy is —COOalkyl wherein alkyl is as described above. Preferred are carbomethoxy and carboethoxy.

The terms use to define compounds of Formulas (9) and (9A) include:

(a) The term “lower alkyl” is a straight or branched group of from 1 to 4 carbons;

(b) The term “alkyl” is a straight or branched group of from 1 to 6 carbon atoms including but not limited to methyl, ethyl, propyl, n-propyl, isopropyl, butyl, 2-butyl, tert-butyl, pentyl, except as where otherwise stated; and

(c) The benzyl and phenyl groups may be unsubstituted or substituted by from 1 to 3 substituents selected from hydroxy, carboxy, carboalkoxy, halogen, CF₃, nitro, alkyl, and alkoxy. Preferred are halogens.

It should be appreciated that the Alpha-2-delta ligand named 3-(1-aminomethyl-cyclohexylmethyl)-4H-[1,2,4]oxadiazol-5-one hydrochloride is also known as “CI-1045”.

As used herein, the phrase “cartilage damage” means a disorder of hyaline cartilage and subchondral bone characterized by hypertrophy of tissues in and around the involved joints, which may or may not be accompanied by deterioration of hyaline cartilage surface. Cartilage damage of OA is thus noninflammatory.

The phrase “treating” means administration of an invention combination as defined above that inhibits the progress, prevents further progress, or reverses progression, in part or in whole, of any one or more symptoms of any one of the diseases and disorders listed above.

The invention combination also includes isotopically-labelled compounds, which are identical to those recited above, but for the fact that one or more atoms are replaced by an atom having an atomic mass or mass number different from the atomic mass or mass number usually found in nature.

Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention and pharmaceutically acceptable salts of said compounds which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above or disclosed in the Schemes and/or in the Examples and Preparations below, by substituting a readily available isotopically labelled reagent for a non-isotopically labelled reagent.

One of ordinary skill in the art will appreciate that the combinations of the invention are useful in treating a diverse array of diseases. One of ordinary skill in the art will also appreciate that when using the combinations of the invention in the treatment of a specific disease that the combinations of the invention may be combined with various existing therapeutic agents used for that disease.

For the treatment of rheumatoid arthritis, the combinations of the invention may be combined with agents such as TNF-α inhibitors such as anti-TNF monoclonal antibodies and TNF receptor immunoglobulin molecules (such as Enbrel®), low dose methotrexate, lefunimide, hydroxychloroquine, d-penicilamine, auranofin or parenteral or oral gold.

The combinations of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib and rofecoxib, analgesics and intraarticular therapies such as corticosteroids and hyaluronic acids such as hyalgan and synvisc.

This invention also relates to a method of or a pharmaceutical composition for treating inflammatory processes and diseases comprising administering a combination of this invention to a mammal, including a human, cat, livestock or dog, wherein said inflammatory processes and diseases are defined as above and said inhibitory combination is used in combination with one or more other therapeutically active agents under the following conditions:

A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;

B.) where a multi-fold treatment of pain and inflammation is desired, said inhibitory combination is administered in combination with inhibitors of other mediators of inflammation, comprising one or more members independently selected from the group consisting essentially of:

(1) NSAIDs;

(2) H₁-receptor antagonists;

(3) kinin-B₁- and B₂-receptor antagonists;

(4) prostaglandin inhibitors selected from the group consisting of PGD-, PGF-PGI₂- and PGE-receptor antagonists;

(5) thromboxane A₂ (TXA₂—) inhibitors;

(6) 5-, 12- and 15-lipoxygenase inhibitors;

(7) leukotriene LTC₄-, LTD4/LTE₄- and LTB₄-inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including coichicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory combination is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-11 converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and

[2] vincristine;

(4) growth hormone secretagogues;

(5) strong analgesics;

(6) local and systemic anesthetics; and

(7) H₂-receptor antagonists, proton pump inhibitors and other gastroprotective agents.

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leukotriene receptor antagonists, IL-1 processing and release inhibitors, ILra, H₁-receptor antagonists; kinin-B₁- and B₂-receptor antagonists; prostaglandin inhibitors such as PGD-, PGF-PGI₂- and PGE-receptor antagonists; thromboxane A₂ (TXA2-) inhibitors; 5- and 12-lipoxygenase inhibitors; leukotriene LTC₄-, LTD4/LTE₄- and LTB₄-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., coichicine, xanthine oxidase inhibitors, e.g., allopurinol and uricosuric agents, e.g., probenecid, sulfinpyrazone and benzbromarone.

The combinations of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The combinations of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The combination of the present invention may also be administered in combination with one or more antibiotic, antifungal, antiprotozoal, antiviral or similar therapeutic agents.

The combinations of the present invention may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The combinations of the present invention may also be used in combination with osteoporosis agents such as roloxifene, lasofoxifene, droloxifene or fosomax and immunosuppressant agents such as FK-506 and rapamycin.

The present invention also relates to the formulation of the combination of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The term “drugs” includes valdecoxib and an Alpha-2-delta ligand, and may further include one or two of the other therapeutic agents described above.

The invention method is useful in human and veterinary medicines for treating mammals suffering from one or more of the above-listed diseases and disorders.

The term “mammal” includes humans, companion animals such as cats and dogs, and livestock animals such as horses, cows, pigs, and sheep.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

All that is required to practice the method of this invention is to administer a combination of valdecoxib and an Alpha-2-delta ligand, or a pharmaceutically acceptable salt thereof, in an amount that is therapeutically effective for preventing, inhibiting, or reversing the condition being treated. The invention combination can be administered directly or in a pharmaceutical composition as described below.

A therapeutically effective amount, or, simply, effective amount, of an invention combination will generally be from about 1 to about 300 mg/kg of subject body weight of valdecoxib and from about 1 to about 300 mg/kg of subject body weight of an Alpha-2-delta ligand, or a pharmaceutically acceptable salt thereof. Typical doses will be from about 10 to about 5000 mg/day for an adult subject of normal weight for each component of the combination. In a clinical setting, regulatory agencies such as, for example, the Food and Drug Administration (“FDA”) in the U.S. may require a particular therapeutically effective amount.

In determining what constitutes an effective amount or a therapeutically effective amount of an invention combination for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including Food and Drug Administration, or an equivalent agency, guidelines, published clinical studies, the subject's (ie, mammal's) age, sex, weight and general condition, as well as the type and extent of the disease, disorder or condition being treated, and the use of other medications, if any, by the subject. As such, the administered dose may fall within the ranges or concentrations recited above, or may vary outside, ie, either below or above, those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of the invention combination that are less than optimum for a particular subject. Thereafter, the dosage can be increased by small increments until the optimum effect under the circumstance is reached. For convenience, the total daily dosage may be divided and administered in portions during the day, if desired.

Pharmaceutical compositions, described briefly here and more fully below, of an invention combination are produced by formulating the invention combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

The percentage of the active ingredients of valdecoxib and an Alpha-2-delta ligand combination in the foregoing compositions can be varied within wide limits, but for practical purposes it is preferably present in a total concentration of at least 10% in a solid composition and at least 2% in a primary liquid composition. The most satisfactory compositions are those in which a much higher proportion of the active ingredients are present, for example, up to about 95%.

Preferred routes of administration of an invention combination are oral or parenteral. For example, a useful intravenous dose is between 5 and 50 mg, and a useful oral dosage is between 20 and 800 mg, both for each of valdecoxib and the Alpha-2-delta ligand. The dosage is within the dosing range used in treatment of the above-listed diseases, or as would be determined by the needs of the patient as described by the physician.

The invention combination may be administered in any form. Preferably, administration is in unit dosage form. A unit dosage form of the invention combination to be used in this invention may also comprise other compounds useful in the therapy of diseases described above. A further description of pharmaceutical formulations useful for administering the invention combinations is provided below.

The advantages of using an invention combination comprising valdecoxib and an Alpha-2-delta ligand which is a compound of Formulas I, II, III, IIIC, IIIF, IIIG, IIIH, IV, (1A), (1B), V, VI, VII, VIII, (9), and (9A), or a pharmaceutically acceptable salt thereof, including gabapentin, pregabalin, 3-(1-aminomethyl-cyclohexylmethyl)-4H-[1,2,4]oxadiazol-5-one hydrochloride, 3-(1-aminomethyl-cycloheptylmethyl)-4H-[1,2,4]oxadiazol-5-one hydrochloride, C-[1-(1H-tetrazol-5-ylmethyl)-cycloheptyl]-methylamine, 3-(2-aminomethyl-4-methyl-pentyl)-4H-[1,2,4]oxadiazol-5-one hydrochloride, (1α,3α,5α)(3-aminomethyl-bicyclo[3.2.0]hept-3-yl)-acetic acid hydrochloride, and (3S,4S)-(1-Aminomethyl-3,4-dimethyl-cyclopentyl)-acetic acid, in a method of the instant invention include the relatively nontoxic nature of the compounds which comprise the combination, their ease of preparation, the fact that the compounds are well-tolerated, and the ease of IV and oral administration of the drugs. Further, typically the Alpha-2-delta ligands are not extensively metabolized in the body.

Another important advantage is that the independent anti-inflammatory and pain reducing properties described above for valdecoxib and Alpha-2-delta ligands may, if desired, allow the amount of traditional NSAID anti-inflammatory agents and/or NSAID pain relieving agent used in the treatment of patients suffering from cartilage damage, arthritis, inflammation and/or pain to be reduced or even eliminated. It is known that NSAID anti-inflammatory and analgesic agents may produce undesirable side effects such as gastrointestinal bleeding and ulceration. These side effects may be reduced or eliminated by using the instant invention to supplement or substitute treatments using NSAID agents.

A further advantage of the invention combination is administration of the combination to treat a disease or disorder in a mammal may allow lower doses of valdecoxib and/or an Alpha-2-delta ligand of the combination to be used than would be used if valdecoxib and the Alpha-2-delta ligand were each administered alone. This advantage is a result of an expected synergistic therapeutic effect for the combination over the sum of the therapeutic effects for each component of the combination administered alone.

A still further advantage is that while it is shown below that Alpha-2-delta ligands alone are useful for treating cartilage damage, and are thus useful for treating the underlying disease pathology of osteoarthritis, it is also known that acute administration (e.g., administration for 5 days or less) of an Alpha-2-delta ligand is typically not effective for immediate relief pain. Chronic administration of Alpha-2-delta ligands, on the other hand, has been shown to be effective at relieving pain. Additionally, it is well known that selective inhibitors of COX-2 such as valdecoxib are effective pain alleviating agents when given acutely or chronically. The invention combination comprising valdecoxib and an Alpha-2-delta ligand would conveniently and valuably provide acute pain relief not available by administration of an Alpha-2-delta ligand alone, and would also provide inhibition of disease progression in osteoarthritis.

Some of the compounds utilized in an invention combination are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in the invention combination.

Pharmaceutically acceptable acid addition salts of the basic compounds useful in the invention combination include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic compound useful in the invention combination is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the compounds and their respective acid addition salt forms are equivalent for purposes of the present invention.

A pharmaceutically acceptable base addition salt of an acidic compound useful in the invention combination may be prepared by contacting the free acid form of the compound with a nontoxic metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of an acidic compound useful in the invention combination may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds useful in the invention combination differ from their respective salt forms somewhat in certain physical properties such as solubility, crystal structure, hygroscopicity, and the like, but otherwise the salts are equivalent to their respective free acid for purposes of the present invention.

Certain of the compounds useful in the invention combination can exist in unsolvated forms as well as solvated forms, including hydrated forms. In general, the solvated forms, including hydrated forms, are equivalent to unsolvated forms and are intended to be encompassed within the scope of the present invention.

Certain of the compounds useful in the invention combination possess one or more chiral centers, and each center may exist in the R or S configuration. An invention combination may utilize any diastereomeric, enantiomeric, or epimeric form of an Alpha-2-delta ligand, or a pharmaceutically acceptable salt thereof, as well as mixtures thereof.

Additionally, certain compounds useful in the invention combination may exist as geometric isomers such as the entgegen (E) and zusammen (Z) isomers of 1,2-disubstituted alkenyl groups or cis and trans isomers of disubstituted cyclic groups. An invention combination may utilize any cis, trans, syn, anti, entgegen (E), or zusammen (Z) isomer of an Alpha-2-delta ligand, or a pharmaceutically acceptable salt thereof, as well as mixtures thereof.

Certain compounds useful in the invention combination can exist as two or more tautomeric forms. Tautomeric forms of the compounds may interchange, for example, via enolization/de-enolization and the like. An invention combination may utilize any tautomeric form of an Alpha-2-delta ligand, or a pharmaceutically acceptable salt thereof, as well as mixtures thereof.

Intermediates for the synthesis of valdecoxib or an Alpha-2-delta ligand, or a pharmaceutically acceptable salt thereof, useful in the invention combination may be prepared by one of ordinary skill in the art of organic chemistry by adapting various synthetic procedures incorporated by reference above or that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods, 1989,by Wiley-Interscience; the text Advanced Organic Chemistry, 4^{th} edition, by Jerry March, Wiley-Interscience, New York, 1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds useful in an invention combination may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A.G., Switzerland, or Lancaster Synthesis Ltd, United Kingdom.

Syntheses of some compounds useful in the invention combination may utilize starting materials, intermediates, or reaction products that contain a reactive functional group. During chemical reactions, a reactive functional group may be protected using protecting groups that render the reactive group substantially inert to the reaction conditions employed. A protecting group is introduced onto a starting material prior to carrying out the reaction step for which a protecting group is needed. Once the protecting group is no longer needed, the protecting group can be removed. It is well within the ordinary skill in the art to introduce protecting groups during a synthesis of valdecoxib or an Alpha-2-delta ligand, or a pharmaceutically acceptable salt thereof, and then later remove them. Procedures for introducing and removing protecting groups are known and referenced such as, for example, in Protective Groups in Organic Synthesis, 2nd ed., Greene T. W. and Wuts P. G., John Wiley & Sons, New York: New York, 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Preparations of valdecoxib or an Alpha-2-delta ligand, or a pharmaceutically acceptable salt thereof, useful in the invention combination are incorporated by reference to the patents or patent application publications described above and to U.S. Provisional Application No. 60/359,295, filed Feb. 22, 2002.

The newly discovered ability of an invention combination to treat diseases and disorders described above, particularly to treat pain, osteoarthritis and inhibit cartilage damage, has been established in animal models as described below.

### Biological Method 1

Induction of Experimental Osteoarthritis in Rabbit (“EOA in Rabbit”)

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water), a combination comprising valdecoxib and gabapentin, or a combination comprising valdecoxib and 3-(1-aminomethyl-cyclohexylmethyl)-4h-[1,2,4]oxadiazol-5-one hydrochloride (10 rabbits per group). Each group is dosed three times per day with the valdecoxib/gabapentin group receiving 20-mg/kg/dose valdecoxib/100-mg gabapentin/kg/dose and the valdecoxib/3-(1-aminomethyl-cyclohexylmethyl)-4h-[1,2,4]oxadiazol-5-one hydrochloride group receiving 20-mg/kg/dose valdecoxib/50-mg3-(1-aminomethyl-cyclohexylmethyl)-4h-[1,2,4]oxadiazol-5-one hydrochloride/kg/dose. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens will also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM are calculated and statistical analysis is done using the Mann-Whitney U-test.

The results of these studies would be expected to show that the valdecoxib/gabapentin test combination reduces cartilage damage, for example, by reducing the size of the lesion on the tibial plateaus. The valdecoxib/3-(1-aminomethyl-cyclohexylmethyl-4H-[1,2,4]oxadiazol-5-one hydrochloride test combination is expected to reduce the damage score for both the femoral condyles and the tibial plateaus. The later test combination also is expected to reduce the lesion size on the plateaus. In support of these observations, the latter combination is also expected to reduce histologic damage. Moreover, both combinations are expected to reduce evidence of synovial changes. In conclusion, results of studies conducted with these combinations would show that valdecoxib/Alpha-2-delta ligand combinations have significant effects on the damage to cartilage and other tissues that occur in this model of cartilage damage. The foregoing study establishes that Alpha-2-delta ligands such as a compound named 3-(1-aminomethyl-cyclohexylmethyl)-4H-[1,2,4]oxadiazol-5-one hydrochloride and gabapentin are effective for the treatment of cartilage damage in human and other mammalian disorders. Such a treatment offers a distinct advantage over existing treatments that only modify pain and other secondary symptoms. The effectiveness of 3-(1-aminomethyl-cyclohexylmethyl)-4H-[1,2,4]oxadiazol-5-one hydrochloride and gabapentin in this model indicates that 3-(1-aminomethyl-cyclohexylmethyl)-4H-[1,2,4]oxadiazol-5-one hydrochloride, gabapentin, and other Alpha-2-delta ligands will have clinically useful effects in preventing and/or treating cartilage damage.

### Biological Method 2

Monosodium Iodoacetate-Induced Osteoarthritis in Rat Model of Cartilage Damage (“MIA Rat”)

Again, one end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions. As it is well known that Alpha-2-delta ligands are not effective for relieving pain when administered in an acute model, such as the instant MIA Rat model, which has a duration of just 14 days, the hind-paw weight distribution effects that are expected to be observed for the invention combination of valdecoxib and an Alpha-2-delta ligand result from the invention combination's ability to provide acute pain relief and directly inhibit damage to cartilage.

Administration of an invention combination in the MIA model is taught by the experiment described below.

An invention combination will relieve pain and inflammation and inhibit cartilage damage:

In the MIA Rat model on Day 0, the hind-paw weight differentials between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either a combination of valdecoxib and an Alpha-2-delta ligand or vehicle (in the instant case, water) daily for 14 days. The combination of valdecoxib and Alpha-2-delta ligand is typically administered at a dose of 30 mg each per kilogram of rat per day (30 mg/kg/day), but may be administered at other doses, such as, for example, doses each independently selected from 10 mg/kg/day, 30 mg/kg/day, 60 mg/kg/day, and 100 mg/kg/day according to the requirements of the compound being studied. It is well within the level of ordinary skill in the pharmaceutical arts to determine a proper dosage of valdecoxib and an Alpha-2-delta ligand in this model. In the instant experiment, administration of the invention combination is optionally by oral administration or intravenous administration via an osmotic pump. After 7 and 14 days, the hind-paw weight distribution is again determined. Typically, the animals administered vehicle alone place greater weight on their unaffected left hind paw than on their right hind paw, while animals administered an invention combination are expected to show a more normal (i.e., more like a healthy animal) weight distribution between their hind paws. Percent inhibition of change in hind paw function is calculated as the percent change in hind-paw weight distribution for treated animals versus control animals:

$$Percent inhibition of change in hind paw function = [ 1 - [ \frac{( Δ {W}_{G} )}{( Δ {W}_{C} )} ] ] \times 100$$

wherein:

ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an invention combination, as measured on Day 14.

The results of the hind-paw weight distribution data are typically presented as “% Inhibition”.

The MIA Rat data expected from the above experiment will establish that the invention combination, including valdecoxib in combination with an Alpha-2-delta ligand selected from gabapentin, 3-(1-aminomethyl-cyclohexylmethyl)-4H-[1,2,4]oxadiazol-5-one hydrochloride, 3-(2-aminomethyl-4-methyl-pentyl)-4H-[1,2,4]-oxadiazol-5-one hydrochloride, 3-(2-amino-1-cyclopentyl-ethyl)-4H-[1,2,4]oxadiazol-5-one hydrochloride, and 3-(1-aminomethyl-cycloheptylmethyl)-4H-[1,2,4]oxadiazol-5-one hydrochloride, are effective at preventing or treating cartilage damage.

In order to measure biochemical or histopathological end points in the MIA Rat model, some of the animals in the above study are then sacrificed, and the amounts of free proteoglycan in both the osteoarthritic right knee joint and the contralateral left knee joint are determined by biochemical analysis. The amount of free proteoglycan in the contralateral left knee joint provides a baseline value for the amount of free proteoglycan in a healthy joint. The amount of proteoglycan in the osteoarthritic right knee joint in animals further administered an invention combination, and the amounts of proteoglycan in the osteoarthritic right knee joint in animals further administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control.

The results are typically expressed as “Proteoglycan loss (%)” and “Inhibition of Proteoglycan loss (%)”, where the percent inhibition of proteoglycan loss is calculated as {[(proteoglycan loss from joint (%) with vehicle)−(proteoglycan loss from joint with invention combination)]÷(proteoglycan loss from joint (%) with vehicle)}×100.

The MIA Rat data expected above would establish that the invention combinations such as valdecoxib in combination with an Alpha-2-delta ligand selected from 3-(1-aminomethyl-cyclohexylmethyl)-4H-[1,2,4]oxadiazol-5-one hydrochloride and (1α,3α,5α)(3-aminomethyl-bicyclo[3.2.0]hept-3-yl)-acetic acid hydrochloride are effective for the treatment of cartilage damage in mammalian patients, including human.

### Biological Method 3

Selective inhibitors of COX-2 may be identified by screening a test compound in the following assays.

Human in vitro Assays

Human Cell-Based COX-1 Assay:

Human peripheral blood obtained from healthy volunteers can be diluted to 1/10 volume with 3.8% sodium citrate solution. The platelet-rich plasma immediately obtained can be washed with 0.14 M sodium chloride containing 12 mM Tris-HCl (pH 7.4) and 1.2 mM EDTA. Platelets can then be washed with platelet buffer (Hanks buffer (Ca free) containing 0.2% BSA and 20 mM Hepes). Finally, the human washed platelets (HWP) can be suspended in platelet buffer at the concentration of 2.85×10⁸ cells/ml and stored at room temperature until use. The HWP suspension (70 μl aliquots, final 2.0×10⁷ cells/ml) can be placed in a 96-well U bottom plate and 10 μL aliquots of 12.6 mM calcium chloride added. Platelets can be incubated with A23187 (final 10 FM, Sigma) with test compound (0.1-100 FM) dissolved in DMSO (final concentration; less than 0.01%) at 37° C. for 15 minutes. The reaction can be stopped by addition of EDTA (final 7.7 mM) and TxB2 in the supernatant quantitated by using a radioimmunoassay kit (Amersham) according to the manufacturer's procedure.

Human Cell-Based COX-2 Assay:

The human cell based COX-2 assay can be carried out as previously described (Moore et al., Inflamm. Res., 45, 54, 1996). Confluent human umbilical vein endothelial cells (HUVECs, Morinaga) in a 96-well flat bottom plate can be washed with 80 ml of RPMI1640 containing 2% FBS and incubated with hIL-1β (final concentration 300 U/ml, R & D Systems) at 37° C. for 24 hours. After washing, the activated HUVECs can be incubated with test compound (final concentration; 0.1 nM-1M) dissolved in DMSO (final concentration; less than 0.01%) at 37° C. for 20 minutes and stimulated with A23187 (final concentration 30 mM) in Hanks buffer containing 0.2% BSA, 20 mM Hepes at 37° C. for 15 minutes. 6-Keto-PGF_{1α}, stable metabolite of PGI2, in the supernatant can be quantitated by using a radioimmunoassay method (antibody; Preseptive Diagnostics, SPA; Amersham).

Canine in vitro Assays:

The following canine cell based COX 1 and COX-2 assays have been reported in Ricketts et al., Evaluation of Selective Inhibition of Canine Cyclooxygenase 1 and 2 by Carprofen and Other Nonsteroidal Anti-inflammatory Drugs, American Journal of Veterinary Research, 59 (11), 1441-1446.

Protocol for Evaluation of Canine COX-1 Activity:

Test compounds can be solubilized and diluted the day before the assay can be to be conducted with 0.1 mL of DMSO/9.9 mL of Hank's balanced salts solution (HBSS) and stored overnight at 4° C. On the day that the assay can be carried out, citrated blood can be drawn from a donor dog, centrifuged at 190×g for 25 minutes at room temperature and the resulting platelet-rich plasma can then be transferred to a new tube for further procedures. The platelets can be washed by centrifuging at 1500×g for 10 minutes at room temperature. The platelets can be washed with platelet buffer comprising Hank's buffer (Ca free) with 0.2% bovine serum albumin (BSA) and 20 mM HEPES. The platelet samples can then be adjusted to 1.5×10⁷/mL, after which 50 gi of calcium ionophore (A23187) together with a calcium chloride solution can be added to 50 l of test compound dilution in plates to produce final concentrations of 1.7 μM A23187 and 1.26 mM Ca. Then, 100 μl of canine washed platelets can be added and the samples can be incubated at 37° C. for 15 minutes, after which the reaction can be stopped by adding 20 μl of 77 mM EDTA. The plates can then be centrifuged at 2000×g for 10 minutes at 4° C., after which 50 μl of supernatant can be assayed for thromboxane B₂ (TXB₂) by enzyme-immunoassay (EIA). The pg/mL of TXB₂ can be calculated from the standard line included on each plate, from which it can be possible to calculate the percent inhibition of COX-1 and the IC₅₀ values for the test compounds.

Protocol for Evaluation of Canine COX-2 Activity:

A canine histocytoma (macrophage-like) cell line from the American Type Culture Collection designated as DH82, can be used in setting up the protocol for evaluating the COX-2 inhibition activity of various test compounds. There can be added to flasks of these cells 10 μg/mL of LPS, after which the flask cultures can be incubated overnight. The same test compound dilutions as described above for the COX-1 protocol can be used for the COX-2 assay and can be prepared the day before the assay can be carried out. The cells can be harvested from the culture flasks by scraping and 15 can then be washed with minimal Eagle's media (MEM) combined with 1% fetal bovine serum, centrifuged at 1500 rpm for 2 minutes and adjusted to a concentration of 3.2×10⁵ cells/mL. To 50 μl of test compound dilution there can be added 50 μl of arachidonic acid in MEM to give a 10 μM final concentration and there can be added as well 100 μl of cell suspension to give a final concentration of 1.6×10⁵ cells/mL. The test sample suspensions can be incubated for 1 hour and then centrifuged at 1000 rpm for 10 minutes at 4° C, after which 50 μl aliquots of each test compound sample can be delivered to EIA plates. The EIA can be performed for prostaglandin E₂ (PGE₂) and the pg/mL concentration of PGE₂ can be calculated from the standard line included on each plate. From this data it can be possible to calculate the percent inhibition of COX-2 and the IC₅₀ values for the test compounds. Repeated investigations of COX-1 and COX-2 inhibition can be conducted over the course of several months. The results are averaged and a single COX-1:COX-2 ratio is calculated.

Whole blood assays for COX-1 and COX-2 are known in the art such as the methods described in C. Brideau, et al., A Human Whole Blood Assay for Clinical Evaluation of Biochemical Efficacy of Cyclooxygenase Inhibitors, Inflammation Research, Vol. 45, pp. 68-74 (1996). These methods may be applied with feline, canine or human blood as needed.

### Biological Method 4

Carrageenan Induced Foot Edema in Rats

Male Sprague-Dawley rats (5 weeks old, Charles River Japan) can be fasted overnight. A line can be drawn using a marker above the ankle on the right hind paw and the paw volume (V0) can be measured by water displacement using a plethysmometer (Muromachi). Animals can be given orally either vehicle (0.1% methyl cellulose or 5% Tween 80) or a test compound (2.5 ml per 10 g body weight). One hour later, the animals can then be injected intradermally with □-carrageenan (0.1 ml of 1% w/v suspension in saline, Zushikagaku) into right hind paw (Winter et al., Proc. Soc. Exp. Biol. Med., 111, 544, 1962; Lombardino et al., Arzneim. Forsch., 25, 1629, 1975) and three hours later, the paw volume (V3) can be measured and the increase in volume (V3−V0) calculated. Since maximum inhibition attainable with classical NSAIDs is 60-70%, ED₃₀ values can be calculated.

### Biological Method 5

Gastric Ulceration in Rats:

The gastric ulcerogenicity of test compound can be assessed by a modification of the conventional method (Ezer et al., J. Pharm. Pharmacol., 28, 655, 1976; Cashin et al., J. Pharm. Pharmacol., 29, 330-336, 1977). Male Sprague-Dawley rats (5 weeks old, Charles River Japan), fasted overnight, can be given orally either vehicle (0.1% methyl cellulose or 5% Tween 80) or a test compound (1 ml per 100 g body weight). Six hours after, the animals can be sacrificed by cervical dislocation. The stomachs can be removed and inflated with 1% formalin solution (10 ml). Stomachs can be opened by cutting along the greater curvature. From the number of rats that showed at least one gastric ulcer or haemorrhaging erosion (including ecchymosis), the incidence of ulceration can be calculated. Animals did not have access to either food or water during the experiment.

### Biological Method 6

Canine whole blood ex vivo determinations of COX-1 and COX-2 activity inhibition The in vivo inhibitory potency of a test compound against COX-1 and COX-2 activity may be evaluated using an ex vivo procedure on canine whole blood. Three dogs can be dosed with 5 mg/kg of the test compound administered by oral gavage in 0.5% methylcellulose vehicle and three dogs can be untreated. A zero-hour blood sample can be collected from all dogs in the study prior to dosing, followed by 2- and 8-hour post-dose blood sample collections. Test tubes can be prepared containing 2 μL of either (A) calcium ionophore A23187 giving a 50 μM final concentration, which stimulates the production of thromboxane B₂ (TXB₂) for COX-1 activity determination; or of (B) lipopolysaccharide (LPS) to give a 10 μg/mL final concentration, which stimulates the production of prostaglandin E₂ (PGE₂) for COX-2 activity determination. Test tubes with unstimulated vehicle can be used as controls. A 500 μL sample of blood can be added to each of the above-described test tubes, after which they can be incubated at 37° C. for one hour in the case of the calcium ionophore-containing test tubes and overnight in the case of the LPS-containing test tubes. After incubation, 10 μL of EDTA can be added to give a final concentration of 0.3%, in order to prevent coagulation of the plasma which sometimes occurs after thawing frozen plasma samples. The incubated samples can be centrifuged at 4° C. and the resulting plasma sample of −200 μL can be collected and stored at −20° C. in polypropylene 96-well plates. In order to determine endpoints for this study, enzyme immunoassay (EIA) kits available from Cayman can be used to measure production of TXB₂ and PGE₂, utilizing the principle of competitive binding of tracer to antibody and endpoint determination by colorimetry. Plasma samples can be diluted to approximate the range of standard amounts which would be supplied in a diagnostic or research tools kit, i.e., 1/500 for TXB₂ and 1/750 for PGE₂.

COX inhibition is observed when the measured percent inhibition is greater than that measured for untreated controls. The percent inhibition in the above table is calculated in a straightforward manner in accordance with the following equation:

$$\% Inhibition ( 2 - hour ) = \frac{( {PGE}_{2} at t = 0 ) - ( {PGE}_{2} at t = 2 )}{( {PGE}_{2} at t = 0 )}$$

Data Analysis:

Statistical program packages, SYSTAT (SYSTAT, INC.) and StatView (Abacus Cencepts, Inc.) for Macintosh can be used. Differences between test compound treated group and control group can be tested for using ANOVA. The IC₅₀ (ED30) values can be calculated from the equation for the log-linear regression line of concentration (dose) versus percent inhibition.

The selective COX-2 inhibitors described above have been, or could have been, identified by at least one of the methods described above and show, or would show, IC₅₀ values of 0.001 μM to 3 μM with respect to inhibition of COX-2 in either the canine or human assays.

As mentioned above, COX-2 selectivity can be determined by ratio in terms of IC₅₀ value of COX-1 inhibition to COX-2 inhibition. In general, it can be said that a compound showing a COX-1/COX-2 inhibition ratio of more than 5 has sufficient COX-2 selectivity.

### Biological Method 7

Carrageenan-Induced Thermal Hyperalgesia in the Rat:

Thermal hyperalgesia was assessed using the rat plantar test (Ugo Basile, Italy) following a modified method of Hargreaves, et al., 1988. Rats were habituated to the apparatus which consisted of three individual perspex boxes on an elevated glass table. A mobile radiant heat source located under the table was focused onto the desired paw and paw withdrawal latencies (“PWL”) recorded. PWL were taken 3 times for both hind paws of each animal, the mean of which represented baselines for right and left hind paws. At least 5 minutes were allowed between each PWL for an animal. The apparatus was calibrated to give a PWL of approximately 10 s. There was an automatic cutoff point of 20 s to prevent tissue damage. After baseline PWLs were determined, animals received an intraplantar injection of carrageenan (100 mL of 20 mg/mL) into the right hind paw. PWLs were reassessed following the same protocol as above 2-hour post-carrageenan (this time point represented the start of peak hyperalgesia) to ascertain that hyperalgesia had developed. Test compounds were administered orally (in a volume of 1 mL/kg) at 2.5 hours after carrageenan. PWLs were reassessed at various times after drug administration.

Administration of an invention combination to a mammal to treat the diseases listed above is preferably, although not necessarily, accomplished by administering the combination in a pharmaceutical dosage form.

The combinations of the present invention can be prepared and administered in a wide variety of oral and parenteral dosage forms. Thus, the combinations of the present invention can be administered by injection, that is, intravenously, intramuscularly, intracutaneously, subcutaneously, intraduodenally, or intraperitoneally. Also, the combinations of the present invention can be administered by inhalation, for example, intranasally. Additionally, the combinations of the present invention can be administered transdermally. It will be obvious to those skilled in the art that the following dosage forms may comprise as the active components, either a compound, or a corresponding pharmaceutically acceptable salt of the compound. The active compounds generally are present in a concentration of about 5% to about 95% by weight of the formulation.

For preparing pharmaceutical compositions from the combinations of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active components are mixed with the carrier having the necessary binding properties in suitable proportions and compacted in the shape and size desired.

The powders and tablets preferably contain from about 5% to about 70%, total, of the active compounds. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compounds with encapsulating material as a carrier providing a capsule in which the active components, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active components are dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active components in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active components, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active components. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active components in a unit dose preparation may be varied or adjusted from 0.01 to 1000 mg, preferably 1 to 100 mg according to the particular application and the potency of the active components. The composition can, if desired, also contain other compatible therapeutic agents.

In therapeutic use as agents to treat the above-listed diseases, the combinations utilized in the pharmaceutical method of this invention are administered at a dose that is effective for treating at least one symptom of the disease or disorder being treated. The initial dosage of about 1 mg/kg to about 100 mg/kg daily of each active component of the invention combination will be effective. A daily dose range of about 25 mg/kg to about 75 mg/kg of each active component is preferred. The dosages, however, may be varied depending upon the requirements of the patient, the severity of the condition being treated, and the combination being employed. Determination of the proper dosage for a particular situation is within the skill of the art. Generally, treatment is initiated with smaller dosages which are less than the optimum dose of the combination. Thereafter, the dosage is increased by small increments until the optimum effect under the circumstance is reached. For convenience, the total daily dosage may be divided and administered in portions during the day if desired. Typical dosages will be from about 0.1 mg/kg to about 500 mg/kg, and ideally about 25 mg/kg to about 250 mg/kg, such that it will be an amount which is effective to treat the particular disease being treated.

A preferred composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A preferred composition provides delayed-, sustained- and/or controlled-release of the selective COX-2 inhibitor, or a pharmaceutically acceptable salt thereof, and/or Alpha-2-delta ligand, or a pharmaceutically acceptable salt thereof. Such preferred compositions include all such dosage forms which produce ≧80% inhibition of COX-2 isozyme activity and ≧80% inhibition of alpha-2-delta binding, and result in a plasma concentration of the active components of the invention combinations of at least 3 fold the COX-2 IC₅₀ and alpha-2-delta binding IC₅₀ for at least 2 hours; preferably for at least 4 hours; preferably for at least 8 hours; more preferably for at least 12 hours; more preferably still for at least 16 hours; even more preferably still for at least 20 hours; and most preferably for at least 24 hours. Preferably, there is included within the above-described dosage forms those which produce ≧80% inhibition of COX-2 isozyme activity and ≧80% inhibition of alpha-2-delta binding, and result in a plasma concentration of the active components of the invention combination of at least 5 fold the active components respective IC₅₀'s for at least 2 hours, preferably for at least 2 hours, preferably for at least 8 hours, more preferably for at least 12 hours, still more preferably for at least 20 hours and most preferably for at least 24 hours. More preferably, there is included the above-described dosage forms which produce ≧90% inhibition of COX-2 isozyme activity and ≧90% inhibition of alpha-2-delta binding, and result in a plasma concentration of the active components of the invention combination of at least 5 fold the active components respective IC₅₀'s for at least 2 hours, preferably for at least 4 hours, preferably for at least 8 hours, more preferably for at least 12 hours, still more preferably for at least 20 hours and most preferably for at least 24 hours.

The following examples illustrate the invention pharmaceutical compositions containing an invention combination and a pharmaceutically acceptable carrier, diluent, or excipient. The examples are representative only, and are not to be construed as limiting the invention in any respect.

FORMULATION EXAMPLE 1
Tablet Formulation:

| Ingredient | Amount (mg) |
|---|---|
| 3-(1-aminomethyl-cyclohexylmethyl)-4H- | 25 |
| [1,2,4]oxadiazol-5-one hydrochloride | |
| Valdecoxib | 20 |
| Lactose | 50 |
| Cornstarch (for mix) | 10 |
| Cornstarch (paste) | 10 |
| Magnesium stearate (1%) | 5 |
| Total | 120 |

3-(1-Aminomethyl-cyclohexylmethyl)-4H-[1,2,4]oxadiazol-5-one hydrochloride, valdecoxib, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases.

### FORMULATION EXAMPLE 2

Coated Tablets:

The tablets of Formulation Example 1 are coated in a customary manner with a coating of sucrose, potato starch, talc, tragacanth, and colorant.

### FORMULATION EXAMPLE 3

Injection vials:

The pH of a solution of 250 g of valdecoxib, 500 g of gabapentin, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 12.5 mg of valdecoxib and 25 mg of gabapentin.

### FORMULATION EXAMPLE 4

Suppositories:

A mixture of 50 g of valdecoxib, 25 g of (α,3α,5α)(3-aminomethyl-bicyclo[3.2.0]hept-3-yl)-acetic acid hydrochloride, 100 g of soya lecithin, and 1400 g of cocoa butter is fused, poured into molds, and allowed to cool. Each suppository contains 50 mg of valdecoxib and 25 mg of (1α,3α,5α)(3-aminomethyl-bicyclo[3.2.0]hept-3-yl)-acetic acid hydrochloride.

### FORMULATION EXAMPLE 5

Solution:

A solution is prepared from 0.5 g of valdecoxib, 1 g of 3-(2-aminomethyl-4-methyl-pentyl)-4H-[1,2,4]-oxadiazol-5-one hydrochloride, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 12.5 mg of valdecoxib and 25 mg of 3-(2-aminomethyl-4-methyl-pentyl)-4H-[1,2,4]-oxadiazol-5-one hydrochloride.

### FORMULATION EXAMPLE 6

Ointment:

100 mg of valdecoxib, 500 mg of 3-(1-aminomethyl-cycloheptylmethyl)-4H-[1,2,4]oxadiazol-5-one hydrochloride is mixed with 99.4 g of petroleum jelly under aseptic conditions. A 5 g portion of the ointment contains 5 mg of valdecoxib and 25 mg of 3-(1-aminomethyl-cycloheptylmethyl)-4H-[1,2,4]oxadiazol-5-one hydrochloride.

### FORMULATION EXAMPLE 7

Capsules:

2 kg of valdecoxib and 2 kg of 3-(1-aminomethyl-cyclohexylmethyl)-4H-[1,2,4]oxadiazol-5-one hydrochloride are filled into hard gelatin capsules in a customary manner such that each capsule contains 25 mg each of valdecoxib and 3-(1-aminomethyl-cyclohexylmethyl)-4H-[1,2,4]oxadiazol-5-one hydrochloride.

### FORMULATION EXAMPLE 8

Ampoules:

A solution of 2.5 kg of valdecoxib and 2.5 kg of gabapentin is dissolved in 60 L of double-distilled water. The solution is sterile filtered, and the filtrate is filled into ampoules. The ampoules are lyophilized under sterile conditions and aseptically sealed. Each ampoule contains 25 mg each of valdecoxib and gabapentin.

While it may be desirable to formulate selective inhibitor of COX-2 and an Alpha-2-delta ligand together in one capsule, tablet, ampoule, solution, and the like, for simultaneous administration, it is not necessary for the purposes of practicing the invention methods. The selective inhibitor of COX-2 and the Alpha-2-delta ligand of an invention combination alternatively can each be formulated independently in any form such as, those of any one Formulation Examples 1 to 8, and administered either simultaneously or at different times.

The following examples illustrate the invention pharmaceutical compositions containing discrete formulations of the active components of the invention combinations and a pharmaceutically acceptable carrier, diluent, or excipient. The examples are representative only, and are not to be construed as limiting the invention in any respect.

FORMULATION EXAMPLE 9
Tablet Formulation of CI-1045:

| Ingredient | Amount (mg) |
|---|---|
| 3-(1-aminomethyl-cyclohexylmethyl)-4H- | 25 |
| [1,2,4]oxadiazol-5-one hydrochloride | |
| Lactose | 50 |
| Cornstarch (for mix) | 10 |
| Cornstarch (paste) | 10 |
| Magnesium stearate (1%) | 5 |
| Total | 100 |

3-(1-Aminomethyl-cyclohexylmethyl)-4H-[1,2,4]oxadiazol-5-one hydrochloride, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet.

Injection Vial Formulation of Valdecoxib:

The pH of a solution of 500 g of valdecoxib and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of valdecoxib.

Such tablets containing Cl-1045 can be administered to a human from one to four times a day for treatment of the above-listed diseases, and the injection solutions containing valdecoxib can be administered to a human 1 or 2 times per day, wherein the administration by injection is optionally simultaneous with administration of the tablets or at different times, for the treatment of one of the above-listed diseases.

### FORMULATION EXAMPLE 10

Coated Tablets Containing CI-1045:

The tablets of Formulation Example 9 are coated in a customary manner with a coating of sucrose, potato starch, talc, tragacanth, and colorant.

Capsules Containing Valdecoxib:

2 kg of valdecoxib are filled into hard gelatin capsules in a customary manner such that each capsule contains 25 mg of valdecoxib.

Such coated tablets containing CI-1045 can be administered to a human from one to four times a day for treatment of the above-listed diseases, and the capsules containing valdecoxib can be administered to a human 1 or 2 times per day, wherein the administration of the capsules is optionally simultaneous with administration of the tablets or at different times, for the treatment of one of the above-listed diseases.

### FORMULATION EXAMPLE 11

The formulation of any one of Formulation Examples 1 to 10, wherein the Alpha-2-delta ligand recited therein is replaced with a compound named (3S,4S)-(1-Aminomethyl-3,4-dimethyl-cyclopentyl)-acetic acid.

### FORMULATION EXAMPLE 12

The formulation of any one of Formulation Examples 1 to 10, wherein the Alpha-2-delta ligand recited therein is replaced with a compound named [(1R,5R,6S)-6-(Aminomethyl)bicyclo[3.2.0]hept-6-yl]acetic acid.

Still further, it should be appreciated that the invention methods comprising administering an invention combination to a mammal to treat diseases or disorders listed above may be used to treat different diseases simultaneously. For example, administration of a selective COX-2 inhibitor in combination with an Alpha-2-delta ligand in accordance with the invention combination may be carried out as described above to treat both inflammation and convulsions in a mammal in need of both treatments.

As shown above, the invention combination offers a distinct advantage over existing treatments for the above-listed diseases, especially those associated with symptoms such as inflammation, pain, cartilage damage, and convulsions.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, in any of the above embodiments, preferred embodiments, and examples wherein valdecoxib is specifically described, it is within the scope of the instant invention to utilize any selective inhibitor of COX-2, including, but not limited to, celecoxib and rofecoxib, in place of valdecoxib. It is intended, therefore, that the invention be defined by the scope of the claims that follow and that such claims be interpreted as broadly as is reasonable.

Having described the invention combinations and their uses, various embodiments of the invention are hereupon claimed.

What is claimed is:

1. A combination, comprising valdecoxib, or a pharmaceutically acceptable salt thereof, and an Alpha-2-delta ligand, or a pharmaceutically acceptable salt thereof, that is not a compound of Formulas 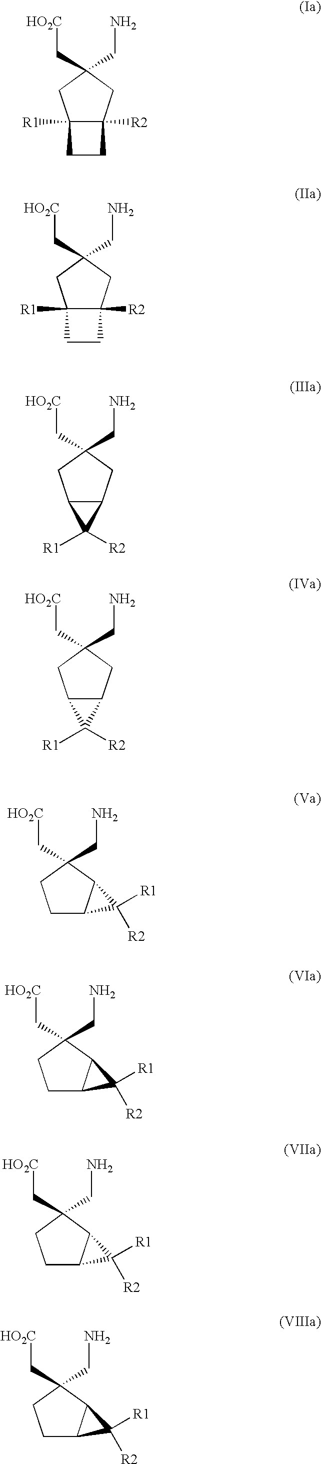 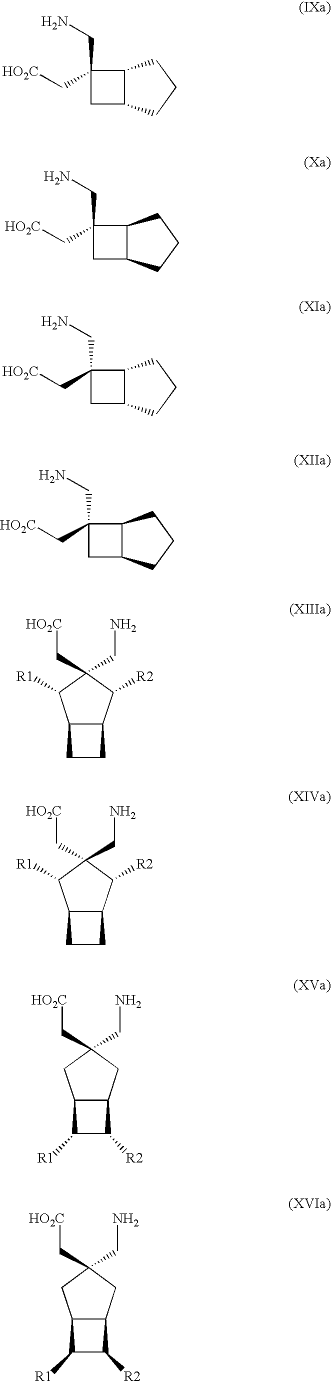 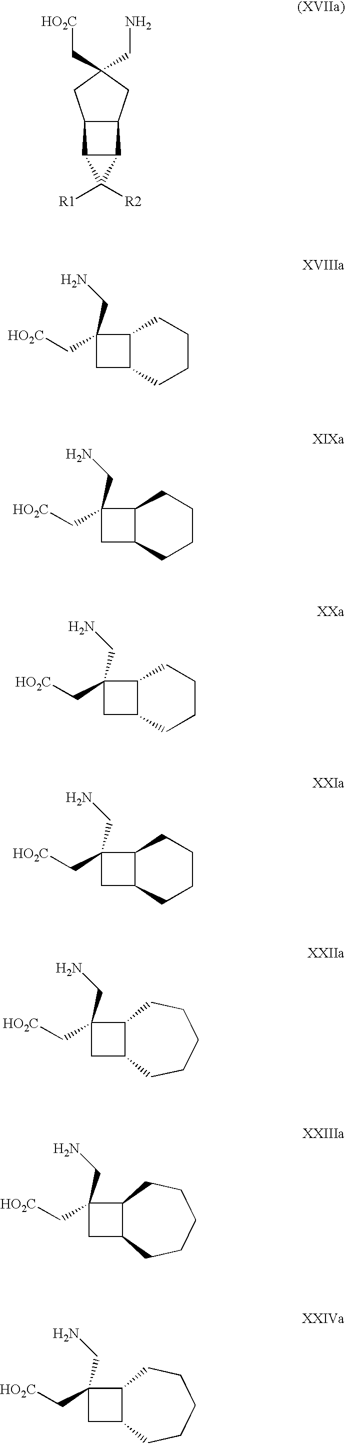 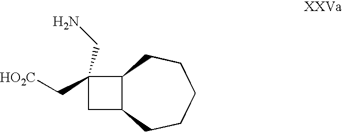
wherein R¹ and R² are each independently selected from H, straight or branched alkyl of 1-6 carbon atoms, cycloalkyl of from 3-6 carbon atoms, phenyl and benzyl, wherein R and R may not each simultaneously be hydrogen except in the case of the compound of formula (XVIIa).

2. The combination according to claim 1, wherein the Alpha-2-delta ligand is gabapentin.

3. The combination according to claim 1, wherein the Alpha-2-delta ligand is pregabalin.

4. The combination according to claim 1, wherein the Alpha-2-delta ligand is a compound named 3-(1-aminomethyl-cyclohexylmethyl)-4H-[1,2,4]oxadiazol-5-one hydrochloride.

5. The combination according to claim 1, wherein the Alpha-2-delta ligand is a compound named:
(3S,4S)-(1-Aminomethyl-3,4-dimethyl-cyclopentyl)-acetic acid;
or a pharmaceutically acceptable salt thereof.

6. A pharmaceutical composition, comprising a combination according to claim 1, and a pharmaceutically acceptable carrier, diluent, or excipient.

7. The pharmaceutical composition according to claim 6, comprising a combination according to any one of claims 2 to 5, and a pharmaceutically acceptable carrier, diluent, or excipient.

8. A method of treating a disease selected from cartilage damage, inflammation, osteoarthritis, rheumatoid arthritis, psoriatic arthritis, and pain in a mammal in need thereof, comprising administering to the mammal a therapeutically effective amount of a combination according to any one of claims 1 to 5.

9. The method according to claim 8, wherein the disease being treated is cartilage damage.

10. The method according to claim 8, wherein the disease being treated is osteoarthritis.

11. The method according to claim 8, wherein disease being treated is rheumatoid arthritis.
